(19) United States
(12) Patent Application Publication
(10) Pub. No.: US 2014/0343118 A1
(43) Pub. Date: Nov. 20, 2014

(54) METHODS OF TREATMENT USING ARYLCYCLOPROPYLAMINE COMPOUNDS

(71) Applicant: Duke University, Durham, NC (US)

(72) Inventors: Dewey G. McCafferty, Chapel Hill, NC (US); Sunhee Hwang, Durham, NC (US); Julie A. Pollock, Durham, NC (US); David M. Gooden, Durham, NC (US)

(21) Appl. No.: 14/210,848

(22) Filed: Mar. 14, 2014

Related U.S. Application Data
(60) Provisional application No. 61/784,236, filed on Mar. 14, 2013.

(51) Int. Cl. C07C 217/74; A61K 31/135; C07C 211/40; A61K 31/381; A61K 31/145; C07D 317/58; A61K 31/36; C07D 333/20; A61K 45/06; C07C 323/31

(52) U.S. Cl. CPC C07C 217/74; A61K 45/06; A61K 31/135; C07C 211/40; C07C 323/31; A61K 31/145; C07D 317/58; A61K 31/36; C07D 333/20; A61K 31/381
USPC 514/438; 514/647; 514/466; 600/1

(57) ABSTRACT

Described herein are methods of treating breast cancer using arylcyclopropylamine compounds.

### CROSS-REFERENCE TO RELATED APPLICATIONS

This application claims the benefit of U.S. Provisional Application No. 61/784,236, filed Mar. 14, 2013, which is incorporated by reference herein in its entirety.

### STATEMENT REGARDING FEDERALLY SPONSORED RESEARCH

This invention was made with United States Government support awarded by National Institutes of Health, Grant No. 1R01GM087566. The U.S. Government has certain rights in this invention.

### BACKGROUND

Worldwide, breast cancer accounts for over 20% of all cancers (excluding non-melanoma skin cancers) in women. While the overwhelming majority of human cases occur in women, male breast cancer can also occur. There is a continuing need for new anti-cancer agents, particularly those that have fewer toxic side-effects.

### SUMMARY

In one aspect, the disclosure provides a method of treating breast cancer in a subject in need of treatment, comprising administering to the subject an effective amount of a compound of formula (I):

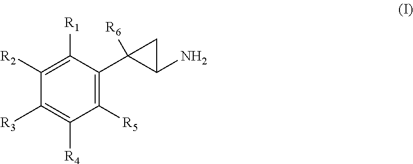

wherein R₁, R₂, R₃, R₄ and R₅ are independently selected from hydrogen, C₁₋₇ alkyl, C₃₋₂₀ heterocyclyl, C₅₋₂₀ aryl, C₁₋₇ alkoxy, C₁₋₇ haloalkyl, halo, amino, cyano, nitro, ether and thioether, or any two of R₁, R₂, R₃, R₄ and R₅ may be taken together with the carbon atoms to which they are attached to form an optionally substituted ring; and
R₆ is selected from hydrogen and optionally substituted C₅₋₂₀ aryl;
or an isomer, prodrug or pharmaceutically acceptable salt thereof

In one aspect, the disclosure provides a method of treating breast cancer in a subject in need of treatment, comprising administering to the subject an effective amount of a compound of formula (II):

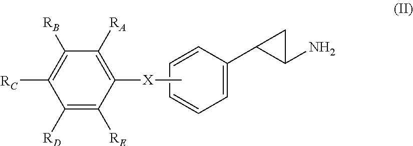

wherein:
X is selected from a bond, O, S, and NH; and
R_{A}, R_{B}, R_{C}, R_{D} and R_{E} are independently selected from hydrogen, C₁₋₇ alkyl, C₅₋₂₀ aryl, C₃₋₂₀ heterocyclyl, C₁₋₇ alkoxy, amino, cyano, nitro, halo, haloalkyl, ether and thioether; or an isomer, prodrug or pharmaceutically acceptable salt thereof.

In another aspect, the disclosure provides a method of treating breast cancer in a subject in need of treatment, comprising administering to the subject an effective amount of a compound of formula (IX):

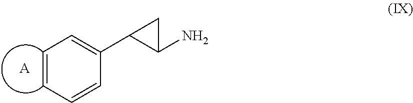

wherein:
A is a C₅-C₆ aryl, cycloalkenyl or heterocyclyl ring.

In another aspect, the disclosure provides a method of treating breast cancer in a subject in need of treatment, comprising administering to the subject an effective amount of a compound of formula (XV):

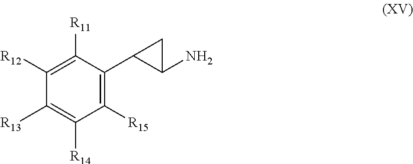

wherein R₁₁, R₁₂, R₁₃, R₁₄ and R₁₅ are independently selected from hydrogen, C₁₋₇ alkyl, C₃₋₂₀ heterocyclyl, C₂₋₇ alkoxy, amino, cyano, nitro, ether and thioether; or an isomer, prodrug or pharmaceutically acceptable salt thereof.

In one aspect, the disclosure provides a method of reducing proliferation of breast cancer cells, comprising contacting the breast cancer cells with an effective amount of a compound of formula (I):

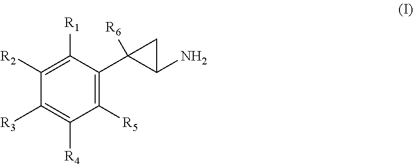

wherein R₁, R₂, R₃, R₄ and R₅ are independently selected from hydrogen, C₁₋₇ alkyl, C₃₋₂₀ heterocyclyl, C₅₋₂₀ aryl, C₁₋₇ alkoxy, C₁₋₇ haloalkyl, halo, amino, cyano, nitro, ether and thioether, or any two of R₁, R₂, R₃, R₄ and R₅ may be taken together with the carbon atoms to which they are attached to form an optionally substituted ring; and
R₆ is selected from hydrogen and optionally substituted C₅₋₂₀ aryl;
or an isomer, prodrug or pharmaceutically acceptable salt thereof

In one aspect, the disclosure provides a method of reducing proliferation of breast cancer cells, comprising contacting the breast cancer cells with an effective amount of a compound of formula (II):

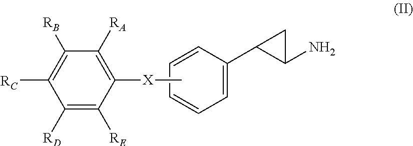

wherein:
X is selected from a bond, O, S, and NH; and
R_{A}, R_{B}, R_{C}, R_{D} and R_{E} are independently selected from hydrogen, C₁₋₇ alkyl, C₅₋₂₀ aryl, C₃₋₂₀ heterocyclyl, C₁₋₇ alkoxy, amino, cyano, nitro, halo, haloalkyl, ether and thioether; or an isomer, prodrug or pharmaceutically acceptable salt thereof.

In another aspect, the disclosure provides a method of reducing proliferation of breast cancer cells, comprising contacting the breast cancer cells with an effective amount of a compound of formula (IX):

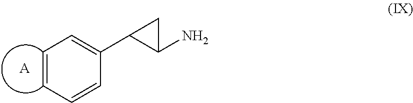

wherein:
A is a C₅-C₆ aryl, cycloalkenyl or heterocyclyl ring.

In another aspect, the disclosure provides a method of reducing proliferation of breast cancer cells, comprising contacting the breast cancer cells with an effective amount of a compound of formula (XV):

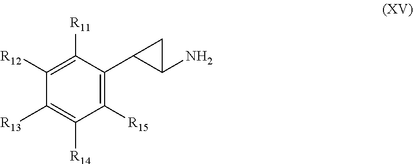

wherein R₁₁, R₁₂, R₁₃, R₁₄ and R₁₅ are independently selected from hydrogen, C₁₋₇ alkyl, C₃₋₂₀ heterocyclyl, C₂₋₇ alkoxy, amino, cyano, nitro, ether and thioether; or an isomer, prodrug or pharmaceutically acceptable salt thereof.

Other aspects and embodiments will become apparent in light of the following disclosure and drawings.

### BRIEF DESCRIPTION OF THE DRAWINGS

FIG. 1 illustrates A. Heatmap illustrating expression levels of FAD-dependent amine oxidases in breast cancer cells lines. B. Heatmap illustrating expression levels of FAD-dependent amine oxidases in breast cancer tumors. Red indicates high expression and blue indicates low expression.

FIG. 2 illustrates expression levels of FAD-dependent amine oxidases in all four cell lines used in these studies. LSD 1 and LSD2 are most highly expressed.

FIG. 3 illustrates knockdown of LSD1 after transfection with either two unique siRNA duplexes to LSD1 (siLSD1 A and siLSD1 B) or siRNA control (siMED). A. mRNA levels measured after treatment for 18 h with either vehicle or 100 nM E2 in MCF7 cells. Data presented as ±SEM. B. mRNA levels measured in MDA-MB-231 cells. Data presented as ±SEM. C. LSD1 protein levels in MCF7 cells. D. LSD1 protein levels in MDA-MB-231 cells. E. MCF7 cell proliferation as measured by total DNA content after knockdown of LSD1. F. MDA-MB-231 cell proliferation as measured by total DNA content after knockdown of LSD1.

FIG. 4 illustrates knockdown of LSD2 does not decrease cellular growth. A. DNA content of MCF7 cells after knockdown of LSD2 for 10 days. B. DNA content of MDA-MB-231 cells after knockdown of LSD2 for 10 days. C. mRNA levels of LSD2 after knockdown with siRNA. D. pS2 mRNA levels. E. PR mRNA levels. All data is represented as ±SEM.

FIG. 5 illustrates inhibition of LSD1 expression of enzymatic activity compromises ERα transcriptional activity. MCF7 cells were transfected with either of two unique siRNA duplexes to LSD1 (siLSD1 A or siLSD1 B) or siRNA control (siMED). After 2 d, the cells were treated for 18 h with either vehicle or 100 nM 17β-estradiol (E2). Activation of genes was also examined for treatment with 1a-c for 6 h followed by vehicle or 100 nM E2 for 18 h. mRNA was analyzed by quantitative RT-PCR for A. pS2, B. GREB1, C. PR and D. EGR1. Data is presented as ±SEM.

FIG. 6 illustrates that after treatment with 1a or 1c or siRNA to LSD1 ERα is recruitment ERE promoters is decreased. A, B. Recruitment of ERα to pS2 ERE and distal promoter. C, D. Recruitment of ERα to two PR EREs and distal promoter. E. Recruitment of LSD1 to pS2 ERE and not distal promoter upon E2 treatment. F. Recruitment of LSD1 to PR ERE and not distal promoter upon E2 treatment. In all cases, IgG is used as a negative control. Data is presented as ±SEM.

FIG. 7 illustrates demethylation of H3K4-Me2 at pS2 and PR promoters is decreased after A. treatment with 1c or B. knockdown with siRNA to LSD 1.

FIG. 8 illustrates proliferation of breast cancer cells after repeated treatment with DMSO control or 250 μM 2-PCPA and 1a-1c. A. ERα-positive MCF7 cells. B. Triple negative MDA-MB-231 cells. C. Triple negative HCC1143 cells. D. Triple negative HCC1937 cells.

FIG. 9 illustrates Representative example of dosage dependent inhibition of proliferation of breast cancer cells after treatment with inhibitors. A. MCF7 cells after treatment with 1c. B. MDA-MB-231 cells after treatment with 1c.

FIG. 10 illustrates cell cycle analysis after treatment with A. 2-PCPA or 1a-c or B. siRNA to LSD1 in MCF7 cells indicates G1 and G2/M arrest.

FIG. 11 illustrates global dimethylation levels of histones from nuclear extracts after treatment with 250 μM 2-PCPA or 1a-1c for 24 h or after knockdown of LSD1 with siRNA. A. MCF7 cells after treatment with inhibitors. B. MDA-MB-231 cells after treatment with inhibitors. C. MCF7 cells after knockdown. D. MDA-MB-231 cells after knockdown.

### DETAILED DESCRIPTION

Before any embodiments of the disclosure are explained in detail, it is to be understood that the methods of the disclosure are not limited in application to the details of construction and the arrangement of components set forth in the following description or illustrated in the following drawings. The methods of the disclosure are capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The invention generally relates to methods of treating breast cancer and related disorders using arylcyclopropylamine compounds.

### DEFINITIONS

The term “C₅₋₂₀ aryl”, as used herein, pertains to a monovalent moiety obtained by removing a hydrogen atom from an aromatic ring atom of a C₅₋₂₀ aromatic compound, said compound having one ring, or two or more rings (e.g., fused), and having from 5 to 20 ring atoms, and wherein at least one of said ring(s) is an aromatic ring. Suitably, each ring has from 5 to 7 ring atoms.

The ring atoms may be all carbon atoms, as in “carboaryl groups”, in which case the group may conveniently be referred to as a “C₅₋₂₀ carboaryl” group. Examples of C₅₋₂₀ aryl groups which do not have ring heteroatoms (i.e. C₅₋₂₀ carboaryl groups) include, but are not limited to, those derived from benzene (i.e. phenyl) (C6), naphthalene (C10), anthracene (C14), phenanthrene (C14), naphthacene (C18), and pyrene (C16).

Examples of aryl groups that comprise fused rings, one of which is not an aromatic ring, include, but are not limited to, groups derived from indene and fluorene.

Alternatively, the ring atoms may include one or more heteroatoms, including but not limited to oxygen, nitrogen, and sulfur, as in “heteroaryl groups”. In this case, the group may conveniently be referred to as a “C₅₋₂₀ heteroaryl” group, wherein “C₅₋₂₀” denotes ring atoms, whether carbon atoms or heteroatoms. Suitably, each ring has from 5 to 7 ring atoms, of which from 0 to 4 are ring heteroatoms. Examples of C₅₋₂₀ heteroaryl groups include, but are not limited to, C5 heteroaryl groups derived from furan (oxole), thiophene (thiole), pyrrole (azole), imidazole (1,3-diazole), pyrazole (1,2-diazole), triazole, oxazole, isoxazole, thiazole, isothiazole, oxadiazole, tetrazole, oxadiazole (furazan) and oxatriazole; and C6 heteroaryl groups derived from isoxazine, pyridine (azine), pyridazine (1,2-diazine), pyrimidine (1,3-diazine; e.g., cytosine, thymine, uracil), pyrazine (1,4-diazine) and triazine.

The above C₅₋₂₀ aryl groups whether alone or part of another substituent, may themselves optionally be substituted with one or more groups selected from themselves and the additional substituents listed below.

The term “C₁₋₇ alkyl”, as used herein, pertains to a monovalent moiety obtained by removing a hydrogen atom from a hydrocarbon compound having from 1 to 7 carbon atoms, which may be aliphatic or alicyclic, or a combination thereof, and which may be saturated, partially unsaturated, or fully unsaturated. Suitably, the alkyl group contains from 3 to 7 carbon atoms, i.e. is a “C₃₋₇ alkyl”.

Examples of saturated linear C₁₋₇ alkyl groups include, but are not limited to, methyl, ethyl, n-propyl, n-butyl, and n-pentyl (amyl).

Examples of saturated branched C₁₋₇ alkyl groups include, but are not limited to, iso-propyl, iso-butyl, sec-butyl, tert-butyl, and neo-pentyl.

Examples of saturated alicyclic C₁₋₇ alkyl groups (also referred to as “C₃₋₇ cycloalkyl” groups) include, but are not limited to, groups such as cyclopropyl, cyclobutyl, cyclopentyl, and cyclohexyl, as well as substituted groups (e.g., groups which comprise such groups), such as methylcyclopropyl, dimethylcyclopropyl, methylcyclobutyl, dimethylcyclobutyl, methylcyclopentyl, dimethylcyclopentyl, methylcyclohexyl, dimethylcyclohexyl, cyclopropylmethyl and cyclohexylmethyl.

Examples of unsaturated C₁₋₇ alkyl groups which have one or more carbon-carbon double bonds (also referred to as “C₂₋₇ alkenyl” groups) include, but are not limited to, ethenyl (vinyl, —CH═CH₂), 2-propenyl (allyl, —CH—CH═CH₂), isopropenyl (—C(CH₃)═CH₂), butenyl, pentenyl, and hexenyl.

Examples of unsaturated C₁₋₇ alkyl groups which have one or more carbon-carbon triple bonds (also referred to as “C₂₋₇ alkynyl” groups) include, but are not limited to, ethynyl and 2-propynyl (propargyl).

Examples of unsaturated alicyclic (carbocyclic) C₁₋₇ alkyl groups which have one or more carbon-carbon double bonds (also referred to as “C₃₋₇ cycloalkenyl” groups) include, but are not limited to, unsubstituted groups such as cyclopropenyl, cyclobutenyl, cyclopentenyl, and cyclohexenyl, as well as substituted groups (e.g., groups which comprise such groups) such as cyclopropenylmethyl and cyclohexenylmethyl.

The term “C₃₋₂₀ heterocyclyl”, as used herein, pertains to a monovalent moiety obtained by removing a hydrogen atom from a ring atom of a C₃₋₂₀ heterocyclic compound, said compound having one ring, or two or more rings (e.g., spiro, fused, bridged), and having from 3 to 20 ring atoms, of which from 1 to 10 are ring heteroatoms, and wherein at least one of said ring(s) is a heterocyclic ring. Suitably, each ring has from 3 to 7 ring atoms, of which from 1 to 4 are ring heteroatoms. Ring heteroatoms may be selected from the group consisting of O, N, S and P. “C₃₋₂₀” denotes ring atoms, whether carbon atoms or heteroatoms.

Examples of C₃₋₂₀ heterocyclyl groups having one nitrogen ring atom include, but are not limited to, those derived from aziridine, azetidine, pyrrolidines (tetrahydropyrrole), pyrroline (e.g., 3-pyrroline, 2,5-dihydropyrrole), 2H-pyrrole or 3H-pyrrole (isopyrrole, isoazole), piperidine, dihydropyridine, tetrahydropyridine, and azepine.

Examples of C₃₋₂₀ heterocyclyl groups having one oxygen ring atom include, but are not limited to, those derived from oxirane, oxetane, oxolane (tetrahydrofuran), oxole (dihydrofuran), oxane (tetrahydropyran), dihydropyran, pyran (C6), and oxepin. Examples of substituted C₃₋₂₀ heterocyclyl groups include sugars, in cyclic form, for example, furanoses and pyranoses, including, for example, ribose, lyxose, xylose, galactose, sucrose, fructose, and arabinose.

Examples of C₃₋₂₀ heterocyclyl groups having one sulfur ring atom include, but are not limited to, those derived from thiirane, thietane, thiolane (tetrahydrothiophene), thiane (tetrahydrothiopyran), and thiepane.

Examples of C₃₋₂₀ heterocyclyl groups having two oxygen ring atoms include, but are not limited to, those derived from dioxolane, dioxane, and dioxepane.

Examples of C₃₋₂₀ heterocyclyl groups having two nitrogen ring atoms include, but are not limited to, those derived from imidazolidine, pyrazolidine (diazolidine), imidazoline, pyrazoline (dihydropyrazole), and piperazine.

Examples of C₃₋₂₀ heterocyclyl groups having one nitrogen ring atom and one oxygen ring atom include, but are not limited to, those derived from tetrahydrooxazole, dihydrooxazole, tetrahydroisoxazole, dihydroisoxazole, morpholine, tetrahydrooxazine, dihydrooxazine, and oxazine.

Examples of C₃₋₂₀ heterocyclyl groups having one oxygen ring atom and one sulfur ring atom include, but are not limited to, those derived from oxathiolane and oxathiane (thioxane).

Examples of C₃₋₂₀ heterocyclyl groups having one nitrogen ring atom and one sulfur ring atom include, but are not limited to, those derived from thiazoline, thiazolidine, and thiomorpholine.

Other examples of C₅₋₂₀ heterocyclic groups (some of which are C₅₋₂₀ heteroaryl groups) which comprise fused rings, include, but are not limited to, C9 heterocyclic groups derived from benzofuran, isobenzofuran, indole, isoindole, purine (e.g., adenine, guanine), benzothiophene, benzimidazole; C10 heterocyclic groups derived from quinoline, isoquinoline, benzodiazine, pyridopyridine, quinoxaline; C13 heterocyclic groups derived from carbazole, dibenzothiophene, dibenzofuran; C14 heterocyclic groups derived from acridine, xanthene, phenoxathiin, phenazine, phenoxazine, phenothiazine.

Other examples of C₃₋₂₀ heterocyclyl groups include, but are not limited to, oxadiazine and oxathiazine.

Examples of heterocyclyl groups which additionally bear one or more oxo (═O) groups, include, but are not limited to, those derived from: C5 heterocyclics, such as furanone, pyrone, pyrrolidone (pyrrolidinone), pyrazolone (pyrazolinone), imidazolidone, thiazolone, and isothiazolone; C6 heterocyclics, such as piperidinone (piperidone), piperidinedione, piperazinone, piperazinedione, pyridazinone, and pyrimidinone (e.g., cytosine, thymine, uracil), and barbituric acid; fused heterocyclics, such as oxindole, purinone (e.g., guanine), benzoxazolinone, benzopyrone (e.g., coumarin); cyclic anhydrides (—C(═O)—O—C(═O)— in a ring), including but not limited to maleic anhydride, succinic anhydride, and glutaric anhydride; cyclic carbonates (—O—C(═O)—O— in a ring), such as ethylene carbonate and 1,2-propylene carbonate; imides (—C(═O)—NR—C(═O)— in a ring), including but not limited to, succinimide, maleimide, phthalimide, and glutarimide; lactones (cyclic esters, —O—C(═O)— in a ring), including, but not limited to, β-propiolactone, γ-butyrolactone, δ-valerolactone (2-piperidone), and ε-caprolactone; lactams (cyclic amides, —NR—C(═O)— in a ring), including, but not limited to, β-propiolactam, γ-butyrolactam (2-pyrrolidone), δ-valerolactam, and ε-caprolactam; cyclic carbamates (—O—C(═O)—NR— in a ring), such as 2-oxazolidone; cyclic ureas (—NR—C(═O)—NR— in a ring), such as 2-imidazolidone and pyrimidine-2,4-dione (e.g., thymine, uracil).

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group, discussed below), a C₃₋₂₀ heterocyclyl group (also referred to as a C₃₋₂₀ heterocyclyloxy group), a C₅₋₂₀ aryl group (also referred to as a C₅₋₂₀ aryloxy group), or a C₅₋₂₀ arylalkyl group (also referred to as a C₅₋₂₀ arylalkyloxy group), for example, a benzyl group.

C₁₋₇ alkoxy: —OR, wherein R is a C₁₋₇ alkyl group. Examples of C₁₋₇ alkoxy groups include, but are not limited to, —OCH₃ (methoxy), —OCH₂CH₃ (ethoxy) and —OC(CH₃)₃ (tert-butoxy).

Oxo (keto, -one): ═O. Examples of cyclic compounds and/or groups having, as a substituent, an oxo group (═O) include, but are not limited to, carbocyclics such as cyclopentanone and cyclohexanone; heterocyclics, such as pyrone, pyrrolidone, pyrazolone, pyrazolinone, piperidone, piperidinedione, piperazinedione, and imidazolidone; cyclic anhydrides, including but not limited to maleic anhydride and succinic anhydride; cyclic carbonates, such as propylene carbonate; imides, including but not limited to, succinimide and maleimide; lactones (cyclic esters, —O—C(═O)— in a ring), including, but not limited to, β-propiolactone, γ-butyrolactone, δ-valerolactone, and ε-caprolactone; and lactams (cyclic amides, —NH—C(═O)— in a ring), including, but not limited to, β-propiolactam, γ-butyrolactam (2-pyrrolidone), δ-valerolactam, and ε-caprolactam.

Imino (imine): ═NR, wherein R is an imino substituent, for example, hydrogen, C₁₋₇ alkyl group, a C₃₋₂₀ heterocyclyl group, or a C₅₋₂₀ aryl group. Examples of ester groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl). Examples of acyl groups include, but are not limited to, —C(═O)CH₃ (acetyl), —C(═O)CH₂CH₃ (propionyl), —C(═O)C(CH₃)₃ (butyryl), and —C(═O)Ph (benzoyl, phenone).

Carboxy (carboxylic acid): —COOH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Amido (carbamoyl, carbamyl, aminocarbonyl, carboxamide): —C(═O)NR1R2, wherein R1 and R2 are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R1 and R2, together with the nitrogen atom to which they are attached, form a heterocyclic structure as in, for example, piperidinocarbonyl, morpholinocarbonyl, thiomorpholinocarbonyl, and piperazinocarbonyl.

Acylamido (acylamino): —NR1C(═O)R2, wherein R1 is an amide substituent, for example, hydrogen, a C₁₋₇ alkyl group, a C₃₋₂₀ heterocyclyl group, or a C₅₋₂₀ aryl group, and R2 is an acyl substituent, for example, a C₁₋₇ alkyl group, a C₃₋₂₀ heterocyclyl group, or a C₅₋₂₀ aryl group. Examples of acylamide groups include, but are not limited to, —NHC(═O)CH₃, —NHC(═O)CH₂CH₃, and —NHC(═O)Ph. R1 and R2 may together form a cyclic structure, as in, for example, succinimidyl, maleimidyl and phthalimidyl.

Acylureido: —N(R1)C(O)NR2C(O)R3 wherein R1 and R2 are independently ureido substituents, for example, hydrogen, a C₁₋₇ alkyl group, a C₃₋₂₀ heterocyclyl group, or a C₅₋₂₀ aryl group. R3 is an acyl group as defined for acyl groups. Examples of acylureido groups include, but are not limited to, —NHCONHC(O)H, —NHCONMeC(O)H, —NHCONEtC(O)H, —NHCONMeC(O)Me, —NHCONEtC(O)Et, —NMeCONHC(O)Et, —NMeCONHC(O)Me, —NMeCONHC(O)Et, —NMeCONMeC(O)Me, —NMeCONEtC(O)Et, and —NMeCONHC(O)Ph.

Carbamate: —NR1-C(O)—OR2 wherein R1 is an amino substituent as defined for amino groups and R2 is an ester group as defined for ester groups. Examples of carbamate groups include, but are not limited to, —NH—C(O)—O-Me, —NMe-C(O)—O-Me, —NH—C(O)—O-Et, —NMe-C(O)—O-t-butyl, and —NH—C(O)—O-Ph.

Thioamido (thiocarbamyl): —C(═S)NR1R2, wherein R1 and R2 are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Tetrazolyl: a five membered aromatic ring having four nitrogen atoms and one carbon atom.

Amino: —NR1R2, wherein R1 and R2 are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, or, in the case of a “cyclic” amino group, R1 and R2, taken together with the nitrogen atom to which they are attached, form a heterocyclic ring having from 4 to 8 ring atoms. Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Imino: ═NR, wherein R is an imino substituent, for example, for example, hydrogen, a C₁₋₇ alkyl group, a C₃₋₂₀ heterocyclyl group, or a C₅₋₂₀ aryl group.

Amidine: —C(═NR)NR₂, wherein each R is an amidine substituent, for example, hydrogen, a C₁₋₇ alkyl group, a C₃₋₂₀ heterocyclyl group, or a C₅₋₂₀ aryl group. An example of an amidine group is —C(═NH)NH₂.

Carbazoyl (hydrazinocarbonyl): —C(O)—NN—R1 wherein R1 is an amino substituent as defined for amino groups. Examples of azino groups include, but are not limited to, —C(O)—NN—H, —C(O)—NN-Me, —C(O)—NN-Et, —C(O)—NN-Ph, and —C(O)—NN—CH₂-Ph.

Nitro: —NO₂.

Nitroso: —NO.

Azido: —N₃.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group. Examples of C₁₋₇ alkylthio groups include, but are not limited to, —SCH₃ and —SCH₂CH₃.

Disulfide: —SS—R, wherein R is a disulfide substituent, for example, a C₁₋₇ alkyl group (also referred to herein as C₁₋₇ alkyl disulfide), a C₃₋₂₀ heterocyclyl group, or a C₅₋₂₀ aryl group. Examples of C₁₋₇ alkyl disulfide groups include, but are not limited to, —SSCH₃ and —SSCH₂CH₃.

Sulfone (sulfonyl): —S(═O)₂R, wherein R is a sulfone substituent, for example, a C₁₋₇ alkyl group, a C₃₋₂₀ heterocyclyl group, or a C₅₋₂₀ aryl group. Examples of sulfone groups include, but are not limited to, —S(═O)₂CH₃ (methanesulfonyl, mesyl), —S(═O)₂CF₃ (triflyl), —S(═O)₂CH₂CH₃, —S(—O)₂C₄F₉ (nonaflyl), —S(—O)₂CH₂CF₃ (tresyl), —S(—O)₂Ph (phenylsulfonyl), 4-methylphenylsulfonyl (tosyl), 4-bromophenylsulfonyl (brosyl), and 4-nitrophenyl (nosyl).

Sulfine (sulfinyl, sulfoxide): —S(═O)R, wherein R is a sulfine substituent, for example, a C₁₋₇ alkyl group, a C₃₋₂₀ heterocyclyl group, or a C₅₋₂₀ aryl group. Examples of sulfine groups include, but are not limited to, —S(═O)CH₃ and —S(═O)CH₂CH₃.

Sulfonyloxy: —OS(═O)₂R, wherein R is a sulfonyloxy substituent, for example, a C₁₋₇ alkyl group, a C₃₋₂₀ heterocyclyl group, or a C₅₋₂₀ aryl group. Examples of sulfonyloxy groups include, but are not limited to, —OS(═O)₂CH₃ and —OS(═O)₂CH₂CH₃.

Sulfinyloxy: —OS(═O)R, wherein R is a sulfinyloxy substituent, for example, a C₁₋₇ alkyl group, a C₃₋₂₀ heterocyclyl group, or a C₅₋₂₀ aryl group. Examples of sulfinyloxy groups include, but are not limited to, —OS(═O)CH₃ and —OS(═O)CH₂CH₃.

Sulfamino: —NR1S(═O)₂OH, wherein R1 is an amino substituent, as defined for amino groups. Examples of sulfamino groups include, but are not limited to, —NHS(═O)₂OH and —N(CH₃)S(═O)₂OH.

Sulfinamino: —NR1S(═O)R, wherein R1 is an amino substituent, as defined for amino groups, and R is a sulfinamino substituent, for example, a C₁₋₇ alkyl group, a C₃₋₂₀ heterocyclyl group, or a C₅₋₂₀ aryl group. Examples of sulfinamino groups include, but are not limited to, —NHS(═O)CH₃ and —N(CH₃)S(═O)C₆H₅.

Sulfamyl: —S(═O)NR1R2, wherein R1 and R2 are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamino: —NR1S(═O)2R, wherein R1 is an amino substituent, as defined for amino groups, and R is a sulfonamino substituent, for example, a C₁₋₇ alkyl group, a C₃₋₂₀ heterocyclyl group, or a C₅₋₂₀ aryl group. Examples of sulfonamino groups include, but are not limited to, —NHS(═O)₂CH₃ and —N(CH₃)S(═O)₂C₆H₅.

Phosphoramidite: —OP(OR1)-N(R2)₂, where R1 and R2 are phosphoramidite substituents, for example, —H, a C₁₋₇ alkyl group, a C₃₋₂₀ heterocyclyl group, or a C₅₋₂₀ aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.

Phosphoramidate: —OP(═O)(OR1)-N(R2)₂, where R1 and R2 are phosphoramidate substituents, for example, —H, a C₁₋₇ alkyl group, a C₃₋₂₀ heterocyclyl group, or a C₅₋₂₀ aryl group. Examples of phosphoramidate groups include, but are not limited to, —OP(═O)(OCH₂CH₃)—N(CH₃)₂, —OP(═O)(OCH₂CH₃)—N(i-Pr)₂, and —OP(═O)(OCH₂CH₂CN)—N(i-Pr)₂.

In many cases, substituents may themselves be substituted. For example, a C₁₋₇ alkoxy group may be substituted with, for example, a C₁₋₇ alkyl (also referred to as a C₁₋₇ alkyl-C₁₋₇ alkoxy group), for example, cyclohexylmethoxy, a C₃₋₂₀ heterocyclyl group (also referred to as a C₅₋₂₀ heterocyclyl-C₁₋₇ alkoxy group), for example phthalimidoethoxy, or a C₅₋₂₀ aryl group (also referred to as a C₅₋₂₀ aryl-C₁₋₇ alkoxy group), for example, benzyloxy.

### Compounds

Compounds that may be used in the methods described herein include compounds of formula (I):

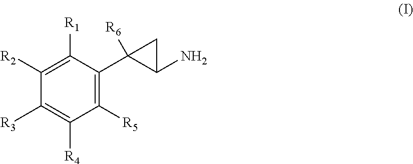

wherein R₁, R₂, R₃, R₄ and R₅ are independently selected from hydrogen, C₁₋₇ alkyl, C₃₋₂₀ heterocyclyl, C₅₋₂₀ aryl, C₁₋₇ alkoxy, C₁₋₇ haloalkyl, halo, amino, cyano, nitro, ether and thioether, or any two of R₁, R₂, R₃, R₄ and R₅ may be taken together with the carbon atoms to which they are attached to form an optionally substituted ring; and
R₆ is selected from hydrogen and optionally substituted C₅₋₂₀ aryl;
or an isomer, prodrug or pharmaceutically acceptable salt thereof.

In some embodiments, at least one of R₁, R₂, R₃, R₄ and R₅ is not hydrogen. In some embodiments, the compound that may be used in the methods described herein has the following formula (Ia):

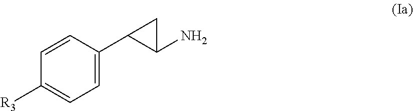

wherein R₃ is selected from hydrogen, C₁₋₇ alkyl, C₃₋₂₀ heterocyclyl, C₅₋₂₀ aryl, C₁₋₇ alkoxy, C₁₋₇ haloalkyl, halo, amino, cyano, nitro, ether and thioether; or an isomer, prodrug or salt thereof

In some embodiments, R³ is halo (e.g., bromo). In some embodiments, R³ is haloalkyl (e.g., trifluoromethyl). In some embodiments, R³ is C₁₋₇ alkoxy (e.g., methoxy, ethoxy or isopropoxy). In some embodiments, R³ is ether (e.g., —O-aryl such as —O-phenyl).

Compounds that may be used in the methods described herein include compounds of formula (II):

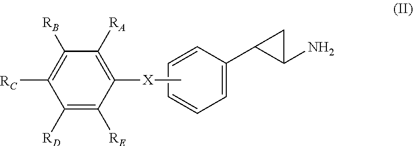

wherein:
X is selected from a bond, O, S, and NH; and
R_{A}, R_{B}, R_{C}, R_{D} and R_{E} are independently selected from hydrogen, C₁₋₇ alkyl, C₅₋₂₀ aryl, C₃₋₂₀ heterocyclyl, C₁₋₇ alkoxy, amino, cyano, nitro, halo, haloalkyl, ether and thioether;

or an isomer, prodrug or pharmaceutically acceptable salt thereof.

In embodiments, compounds that may be used in the methods described herein include compounds of formula (III):

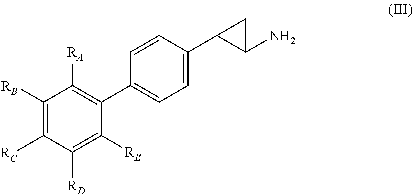

wherein R_{A}, R_{B}, R_{C}, R_{D} and R_{E} are independently selected from hydrogen, C₁₋₇ alkyl, C₅₋₂₀ aryl, C₃₋₂₀ heterocyclyl, C₁₋₇ alkoxy, amino, cyano, nitro, halo, haloalkyl, ether and thioether; or an isomer, prodrug or pharmaceutically acceptable salt thereof.

In embodiments, compounds that may be used in the methods described herein include compounds of formula (IV):

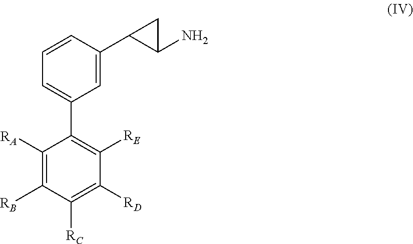

wherein R_{A}, R_{B}, R_{C}, R_{D} and R_{E} are independently selected from hydrogen, C₁₋₇ alkyl, C₅₋₂₀ aryl, C₃₋₂₀ heterocyclyl, C₁₋₇ alkoxy, amino, cyano, nitro, halo, haloalkyl, ether and thioether; or an isomer, prodrug or pharmaceutically acceptable salt thereof.

In embodiments, compounds that may be used in the methods described herein include compounds of formula (V):

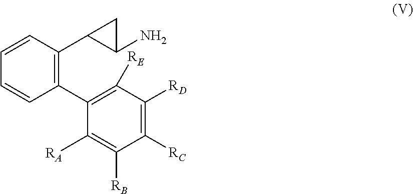

wherein R_{A}, R_{B}, R_{C}, R_{D} and R_{E} are independently selected from hydrogen, C₁₋₇ alkyl, C₅₋₂₀ aryl, C₃₋₂₀ heterocyclyl, C₁₋₇ alkoxy, amino, cyano, nitro, halo, haloalkyl, ether and thioether; or an isomer, prodrug or pharmaceutically acceptable salt thereof.

In embodiments, compounds that may be used in the methods described herein include compounds of formula (VI):

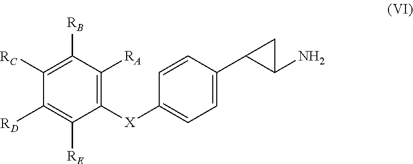

wherein R_{A}, R_{B}, R_{C}, R_{D} and R_{E} are independently selected from hydrogen, C₁₋₇ alkyl, C₅₋₂₀ aryl, C₃₋₂₀ heterocyclyl, C₁₋₇ alkoxy, amino, cyano, nitro, halo, haloalkyl, ether and thioether; and X is selected from O, S, and NH; or an isomer, prodrug or pharmaceutically acceptable salt thereof.

In some embodiments, X is O. In some embodiments, X is S. In some embodiments, R_{C} is C₁₋₇ alkyl such as tert-butyl.

In embodiments, compounds that may be used in the methods described herein include compounds of formula (VII):

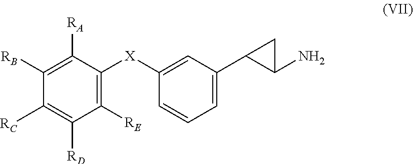

wherein R_{A}, R_{B}, R_{C}, R_{D} and R_{E} are independently selected from hydrogen, C₁₋₇ alkyl, C₅₋₂₀ aryl, C₃₋₂₀ heterocyclyl, C₁₋₇ alkoxy, amino, cyano, nitro, halo, haloalkyl, ether and thioether; and X is selected from O, S, and NH; or an isomer, prodrug or pharmaceutically acceptable salt thereof.

In embodiments, compounds that may be used in the methods described herein include compounds of formula (VIII):

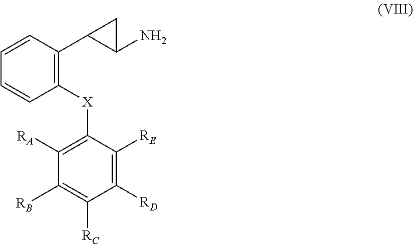

wherein R_{A}, R_{B}, R_{C}, R_{D} and R_{E} are independently selected from hydrogen, C₁₋₇ alkyl, C₅₋₂₀ aryl, C₃₋₂₀ heterocyclyl, C₁₋₇ alkoxy, amino, cyano, nitro, halo, haloalkyl, ether and thioether; and X is selected from a O, S, and NH; or an isomer, prodrug or pharmaceutically acceptable salt thereof.

In embodiments, compounds that may be used in the methods described herein include compounds of formula (IX):

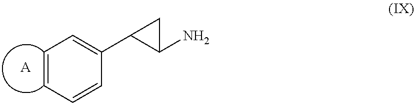

wherein:
A is a C₅-C₆ aryl, cycloalkenyl or heterocyclyl ring;
or an isomer, prodrug or pharmaceutically acceptable salt thereof.

In embodiments, compounds that may be used in the methods described herein include compounds of formula (X):

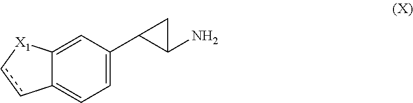

wherein X₁ is selected from CH₂, O, S, and NH; and - - - represents the presence or absence of a bond; or an isomer, prodrug or pharmaceutically acceptable salt thereof.

In embodiments, compounds that may be used in the methods described herein include compounds of formula (XI):

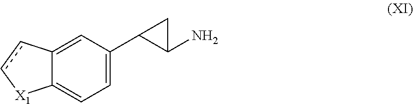

wherein X₁ is selected from CH₂, O, S, and NH; and - - - represents the presence or absence of a bond; or an isomer, prodrug or pharmaceutically acceptable salt thereof.

In embodiments, compounds that may be used in the methods described herein include compounds of formula (XII):

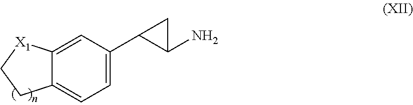

wherein X₁ is selected from CH₂, O, S, and NH; n is 1 or 2; or an isomer, prodrug or pharmaceutically acceptable salt thereof.

In embodiments, compounds that may be used in the methods described herein include compounds of formula (XIII):

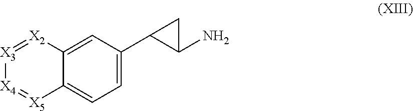

wherein X₂, X₃, X₄ and X₅ are independently selected from CH and N; or an isomer, prodrug or pharmaceutically acceptable salt thereof.

In embodiments, compounds that may be used in the methods described herein include compounds of formula (XIV):

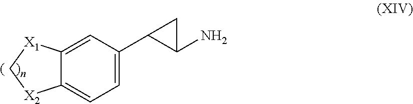

wherein X₁ and X₂ are independently selected from O and S; and n is 1 or 2; or an isomer, prodrug or pharmaceutically acceptable salt thereof.

In some embodiments, X₁ and X₂ are O. In some embodiments, n is 1.

In embodiments, compounds that may be used in the methods described herein include compounds of formula (XV):

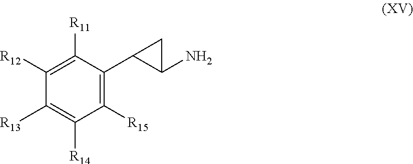

wherein R₁₁, R₁₂, R₁₃, R₁₄ and R₁₅ are independently selected from hydrogen, C₁₋₇ alkyl, C₃₋₂₀ heterocyclyl, C₂₋₇ alkoxy, amino, cyano, nitro, ether and thioether; or an isomer, prodrug or pharmaceutically acceptable salt thereof.

In some embodiments, R₁₃ is C₂₋₇ alkoxy, such as ethoxy or isopropoxy. In some embodiments, R₁₃ is ether, such as phenoxy or benzyloxy. In some embodiments, R₁₃ is amino. In some embodiments, R₁₃ is thioether.

In embodiments, compounds that may be used in the methods described herein include compounds of formula (XVI):

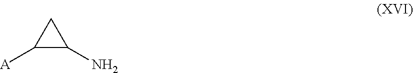

where A is an optionally substituted C₅₋₂₀ aryl group, or an isomer, prodrug or pharmaceutically acceptable salt thereof

Suitable compounds include:

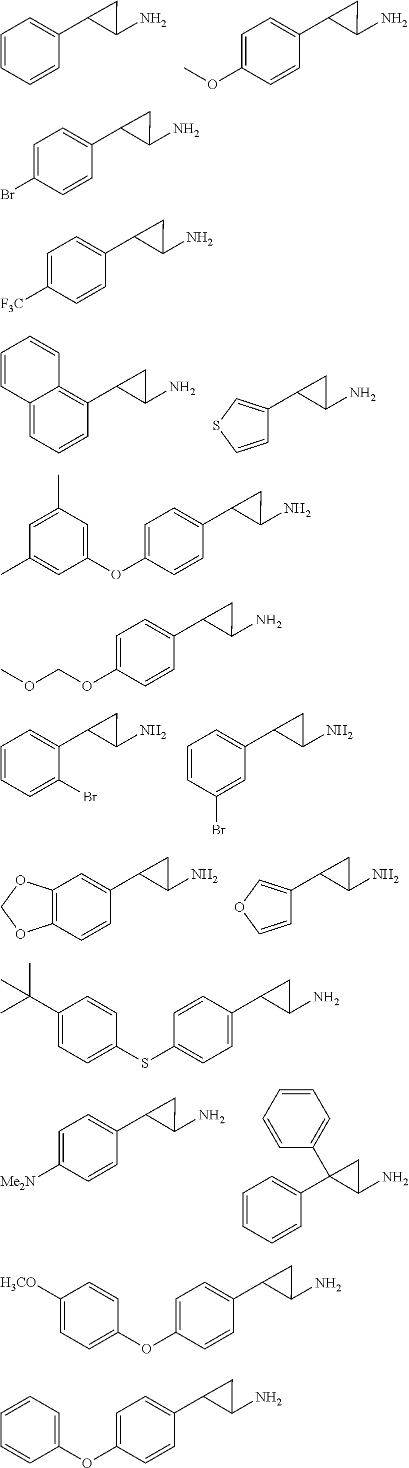

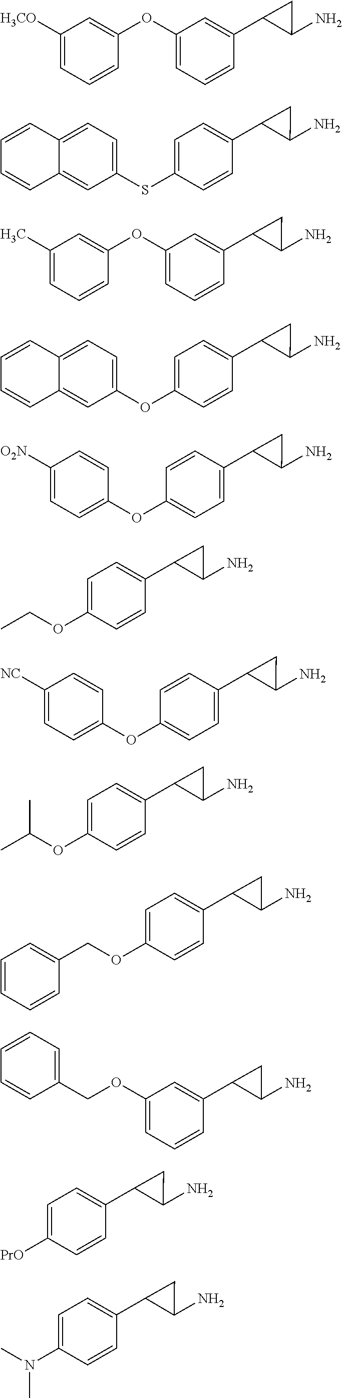

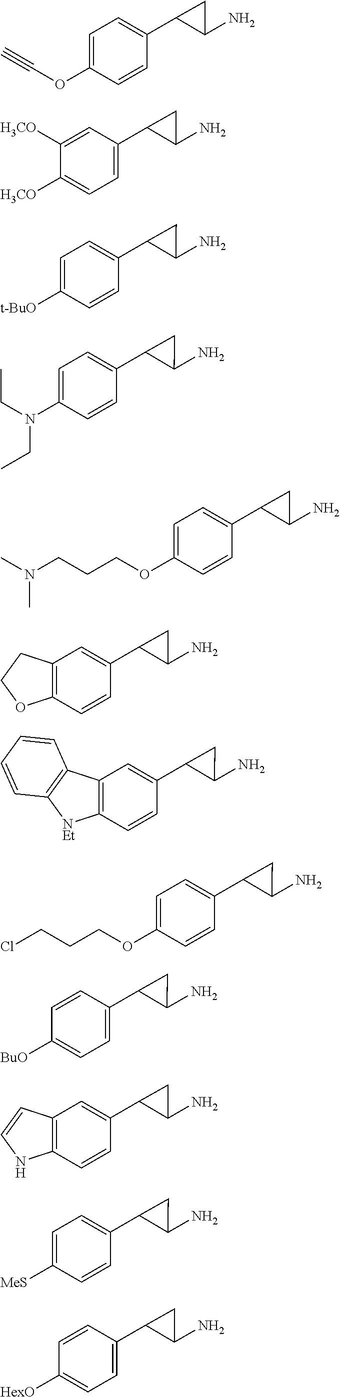

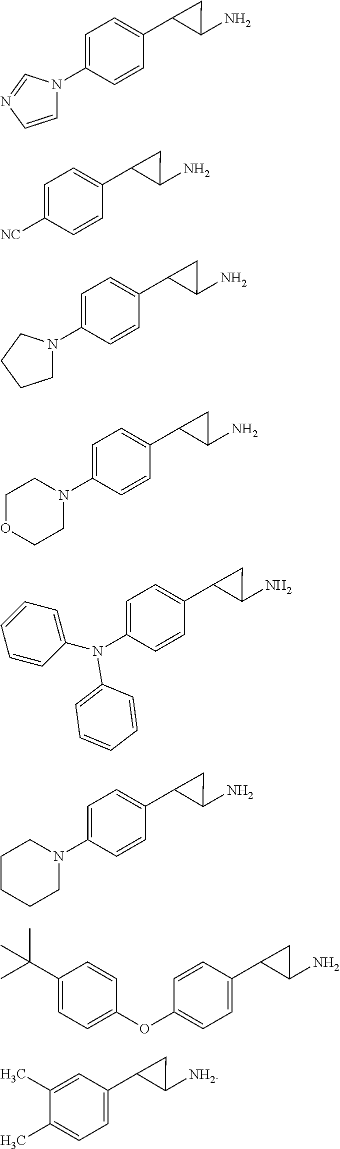

Suitable compounds include those described in U.S. Patent Publication No. 2010/0324147, and in Gooden et al., Bioorg. Med. Chem. Lett. 18 (2008) 3047-3051, each of which is incorporated herein by reference in its entirety.

### Isomers, Salts, Protected Forms, and Prodrugs

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and paramethoxyphenyl).

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g., fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below. It may be convenient or desirable to prepare, purify, and/or handle a corresponding salt of the active compound, for example, a pharmaceutically-acceptable salt. Examples of pharmaceutically acceptable salts are discussed in Berge et al., J. Pharm. Sci., 66, 1-19 (1977). Exemplary pharmaceutically acceptable salts include hydrochloride salts.

For example, if the compound is anionic, or has a functional group which may be anionic (e.g., —COOH may be —COO—), then a salt may be formed with a suitable cation. Examples of suitable inorganic cations include, but are not limited to, alkali metal ions such as Na⁺ and alkaline earth cations such as Ca²⁺ and Mg²⁺, and other cations such as Al³⁺. Examples of suitable organic cations include, but are not limited to, ammonium ion (i.e., NH⁴⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: acetic, propionic, succinic, glycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, phenylsulfonic, toluenesulfonic, methanesulfonic, ethanesulfonic, ethane disulfonic, oxalic, pantothenic, isethionic, valeric, lactobionic, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form”, as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts, Wiley, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc). For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. For example, an amine group may be protected, for example, as an amide or a urethane, for example, as: a methyl amide (—NHCO—CH₃); a benzyloxy amide (—NHCO—OCH₂C₆H₅, —NHCbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH—Fmoc), as a 6-nitroveratryloxy amide (—NH—Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH—Psec); or, in suitable cases, as an N-oxide.

For example, a carboxylic acid group may be protected as an ester for example, as: an C₁₋₇ alkyl ester (e.g. a methyl ester; a t-butyl ester); a C₁₋₇ haloalkyl ester (e.g., a C₁₋₇ trihaloalkylester); a triC₁₋₇ alkylsilyl-C₁₋₇ alkyl ester; or a C₅₋₂₀ aryl-C₁₋₇ alkyl ester (e.g. a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃). It may be convenient or desirable to prepare, purify, and/or handle the active compound in the form of a prodrug.

The term “prodrug”, as used herein, pertains to a compound which, when metabolized (e.g. in vivo), yields the desired active compound. Typically, the prodrug is inactive, or less active than the active compound, but may provide advantageous handling, administration, or metabolic properties.

For example, some prodrugs are esters of the active compound (e.g. a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required. Examples of such metabolically labile esters include those wherein R is C₁₋₇ alkyl (e.g. -Me, -Et); C₁₋₇ aminoalkyl (e.g. aminoethyl; 2-(N,N-diethylamino)ethyl; 2-(4-morpholino)ethyl); and acyloxy-C₁₋₇ alkyl (e.g. acyloxymethyl; acyloxyethyl; e.g. pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexylcarbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl).

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

### Synthesis of Compounds

Compounds of the invention may be synthesized according to Scheme 1. For example, an α,β-unsaturated carboxylic acid may be protecting with an acid protecting group (e.g., as an ester such as a methyl ester). Cyclopropanation may be effected by a number of methods, such as use of the Corey-Chaykovsky reagent, or diazomethane in the presence of a catalyst (e.g., palladium(II) acetate). Subsequent deprotection (e.g., via hydrolysis) may be followed by conversion of the carboxylic acid to a primary amine, e.g., via Curtius rearrangement or a Hofmann rearrangement.

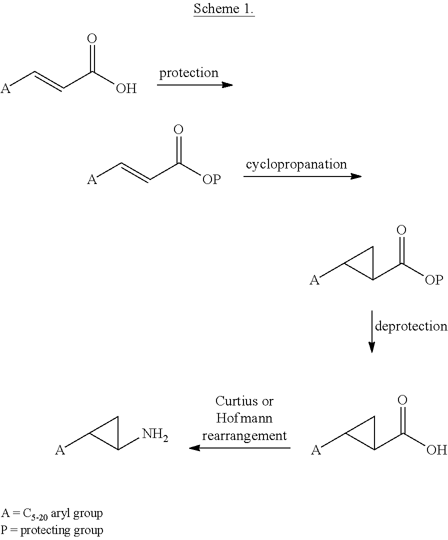

The starting material may be a commercially available α,β-unsaturated acid. Alternatively, an appropriate alkene may be generated from the corresponding aryl aldehyde via an olefination reaction (e.g., the Horner-Wadsworth-Emmons reaction). Additional non-commerically available substituted benzaldehydes for olefination can be prepared using a cross-coupling reaction (e.g., a copper-catalyzed Ullmann coupling) between para-halobenzaldehydes and a variety of phenols and thiophenols, as illustrated in Scheme 2.

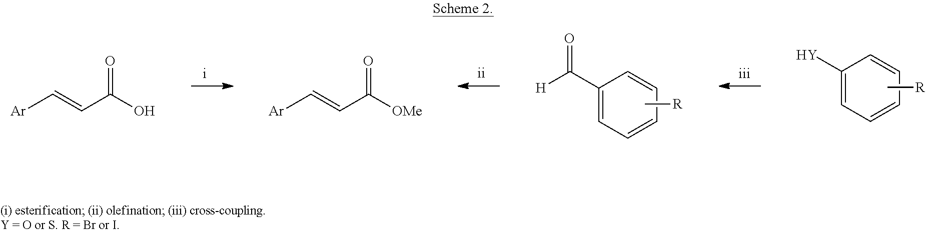

As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCR Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

### Evaluating Compounds

A variety of methods can be used to evaluate a compound for its potential use in treatment of cancer. Evaluation methods include in vitro assays, in vitro cell-based assays, ex vivo assays and in vivo methods. The methods can evaluate binding to a protein or enzyme, an activity downstream of a protein or enzyme of interest, or treatment or alleviation of symptoms.

For example, compounds may be assayed for their ability to inhibit the enzymatic activity of recombinant LSD1 utilizing a horseradish peroxidase coupled assay for the detection of hydrogen peroxide formed in the demethylase catalytic cycle.

Compounds described herein may also be evaluated in other widely accepted animal models of breast cancer.

### Treatment of Breast Cancer

Growth of human breast cancer is closely related to steroid hormones, and the effects of steroid hormones are mediated by their respective receptors. Among steroid hormone receptors, estrogen receptor a (ERα) is of special interest because of the highly increased expression level in malignant breast lesions. Thus, ERα is implicated as a key transcriptional regulator in breast cancer biology. The transcriptional function of ERα is known to be influenced by several coregulatory proteins, some of which possess intrinsic enzymatic activities that modify chromatin structure.

Histone modifications play important roles in regulating gene expression in various cellular processes by altering the underlying chromatin structure and thus influencing related pathological conditions. Histone methylation is one such modification that was thought to be static and enzymatically irreversible until the recent discovery of histone demethylases. Lysine specific demethylase 1 (LSD1) is a unique histone demethylase that removes methyl groups on lysine residues and mediates expression of many genes important in cancer progression. LSD1 activity and its substrate specificity are mainly regulated by association with a number of co-regulatory proteins. LSD1 is one enzyme that removes histone methylation marks in the vicinity of ERα target genes and is known to regulate ERα-mediated transcriptions.

LSD1 demethylation activity can be regulated by its associated proteins. One such protein is CoREST, which has been shown to play an essential role in endowing LSD1 with the ability to demethylate nucleosomal substrates. CoREST binds tightly to LSD1. The dissociation constant for LDS1-CoREST binding is 16 nM. Disruption of the protein-protein interaction leads to a new avenue of therapeutic intervention for breast cancer.

The indolent forms of breast cancer usually have estrogen receptors, but over time, or in aggressive cancers expression of estrogen receptors is lost, yet LSD1 is still over expressed. Surprisingly, the compounds of the present invention also work well against estrogen receptor-negative cancer cell lines.

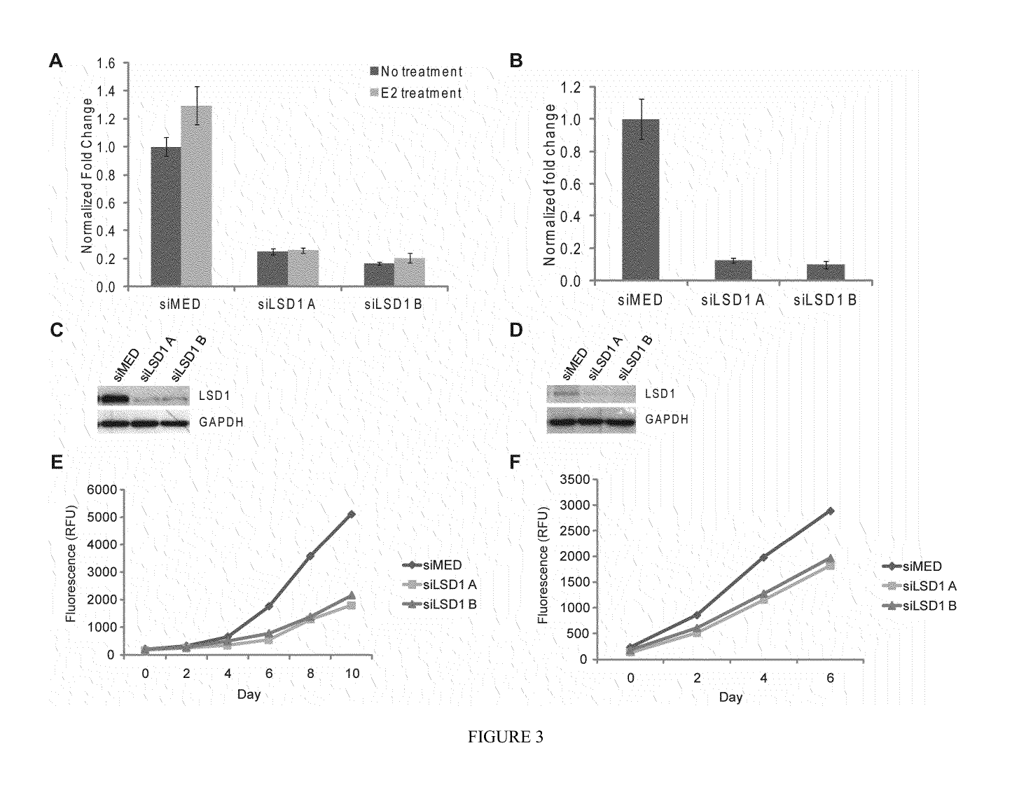

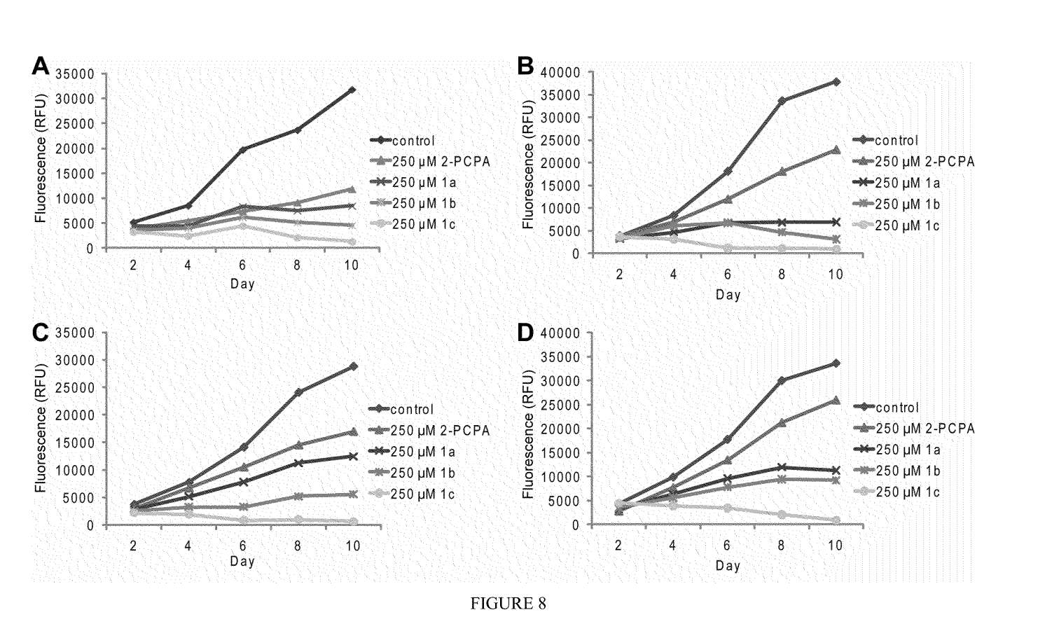

Suitably, the compounds according to the present invention are effective inhibitors of LSD1. LSD1 is essential for the proliferation of both ERα-positive and negative breast cancer cells. 2-PCPA derivatives 1a-1c have a similar but slightly more profound effect on the proliferation of the breast cancer cells (FIG. 8A-B) than the knockdown of LSD1 by siRNA (FIG. 3A-B). Without wishing to be bound by theory, this could be attributed to a few factors. First, the knockdown approach utilized herein does not completely deplete the cellular levels of LSD1 protein, and therefore, active enzyme may be present, albeit at lower levels. Thus, the residual levels could be expected to carry out the essential functions of LSD1. Second, the 2-PCPA derived compounds may be inhibiting other enzymes that are also crucial to breast cancer cell proliferation and survival. However, among the many breast cancer cell lines found to be sensitive to 2-PCPA, LSD1 and LSD2 were the only two FAD dependent amine oxidases highly expressed.

While the specific mechanisms underlying sensitivity to LSD1 inhibition remain to be defined, it is clear that the anti-proliferative activities of the compounds is not secondary to inhibition of ER-transcriptional activity and that this enzyme is involved in additional processes fundamental for proliferation. Despite the important role of LSD1, LSD2 may have a different cellular responsibility in breast cancer or be less important for contributing to proliferative effects in breast cancers. Although both enzymes catalyze similar chemical reactions, their sequences and the presence of conserved protein-protein interaction domains (such as the Tower domain in LSD1 that recruits CoREST) suggests that there are clearly structural differences that might contribute to differing functional roles in the cell. Also, there is a strong possibility that additional components of respective LSD1 and LSD2 complexes may facilitate target selection and specificity. Nonetheless it is clear that LSD1 plays a critical role in ERα signaling.

In an aspect, the disclosure provides a method of treating breast cancer in a subject in need of treatment, comprising administering to the subject an effective amount of a compound of any of formulae (I)-(XVI) as described herein.

In another aspect, the disclosure provides a method of treating breast cancer in a subject in need of treatment, comprising administering to the subject an effective amount of a compound of any of formulae (I)-(XVI) as described herein, and another cancer therapy.

In an aspect, the disclosure provides a method of treating estrogen receptor-negative breast cancer in a subject in need of treatment, comprising administering to the subject an effective amount of a compound of any of formulae (I)-(XVI) as described herein.

“Contacting,” as used herein as in “contacting a cell,” refers to contacting a cell directly or indirectly in vitro, ex vivo, or in vivo (i.e. within a subject, such as a mammal, including humans, mice, rats, rabbits, cats, and dogs). Contacting a cell, can occur as a result of administration to a subject. Contacting encompasses administration to a cell, tissue, mammal, subject, patient, or human. Further, contacting a cell includes adding an agent to a cell culture. Other suitable methods may include introducing or administering an agent to a cell, tissue, mammal, subject, or patient using appropriate procedures and routes of administration as defined herein.

The term “effective amount” as used herein, pertains to that amount of an active compound, or a material, composition or dosage from comprising an active compound, which produces some desired effect, such as alleviation of symptoms or alleviation of side effects. This term as used herein may also refer to an amount effective at bringing about a desired in vivo effect in an animal, mammal, or human, such as reducing proliferation of a cancer cell.

“Reducing proliferation of a cell,” as used herein, refers to reducing, inhibiting, or preventing the survival, growth, or differentiation of a cell, including killing a cell. A cell can be derived from any organism or tissue type and includes, for example, a cancer cell (e.g., neoplastic cells, tumor cells, and the like).

The term “treatment”, as used herein in the context of treating a condition, pertains generally to treatment and therapy, whether of a human or an animal (e.g. in veterinary applications), in which a desired therapeutic effect is achieved. For example, treatment may ameliorate the condition or may inhibit the progress of the condition (e.g., reduce the rate of progress or halt the rate of progress), or may alleviate symptoms of the condition.

The term “therapeutically-effective amount” as used herein, pertains to that amount of an active compound, or a material, composition or dosage form comprising an active compound, which is effective for producing some desired therapeutic effect, commensurate with a reasonable benefit/risk ratio.

### Other Anti-Cancer Agents

Compounds described herein can be used in conjunction with other anti-cancer/chemotherapeutic agents. Exemplary anti-cancer/chemotherapeutic agents include, but are not limited to, the following:

alkylating agents (including, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), bendamustine (Treakisym®, Ribomustin®, Treanda®) chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexylen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), estramustine (Emcyt®, Estracit®), fotemustine, irofulven, mannosulfan, mitobronitol, nimustine, procarbazine, ranimustine, semustine, triaziquone, treosulfan, and Dacarbazine (DTIC-Dome®).

anti-EGFR antibodies (e.g., cetuximab (Erbitux®), panitumumab (Vectibix®), and gefitinib (Iressa®)).

anti-Her-2 antibodies (e.g., trastuzumab (Herceptin®) and other antibodies from Genentech).

antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), carmofur, cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®), decitabine (Dacogen®), enocitabine (Sunrabin®), sapacitabine, tegafur-uracil, tiazofurine, tioguanine, trofosfamide, and gemcitabine (Gemzar®).

vinca alkaloids: vinblastine (Velban®, Velsar®), vincristine (Vincasar®, Oncovin®), vindesine (Eldisine®), vinorelbine (Navelbine®), vinflunine (Javlor®).

platinum-based agents: carboplatin (Paraplat®, Paraplatin®), cisplatin (Platinol®), oxaliplatin (Eloxatin®), nedaplatin, satraplatin, triplatin.

anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®), aclarubicin, amrubicin, liposomal doxorubicin, liposomal daunorubicin, pirarubicin, pixantrone, zorubicin.

topoisomerase inhibitors: topotecan (Hycamtin®), irinotecan (Camptosar®), etoposide (Toposar®, VePesid®), teniposide (Vumon®), lamellarin D, SN-38, camptothecin (e.g., IT-101), belotecan, rubitecan.

taxanes: paclitaxel (Taxol®), docetaxel (Taxotere®), larotaxel, cabazitaxel, ortataxel, tesetaxel.

antibiotics: actinomycin (Cosmegen®), bleomycin (Blenoxane®), hydroxyurea (Droxia®, Hydrea®), mitomycin (Mitozytrex®, Mutamycin®).

immunomodulators: lenalidomide (Revlimid®), thalidomide (Thalomid®).

immune cell antibodies: alemtuzamab (Campath®), gemtuzumab (Myelotarg®), rituximab (Rituxan®), tositumomab (Bexxar®).

interferons (e.g., IFN-alpha (Alferon®, Roferon-A®, Intron®-A) or IFN-gamma (Actimmune®)).

interleukins: IL-1, IL-2 (Proleukin®), IL-24, IL-6 (Sigosix®), IL-12.

HSP90 inhibitors (e.g., geldanamycin or any of its derivatives). In certain embodiments, the HSP90 inhibitor is selected from geldanamycin, 17-alkylamino-17-desmethoxygeldanamycin (“17-AAG”) or 17-(2-dimethylaminoethyl)amino-17-desmethoxygeldanamycin (“17-DMAG”).

anti-androgens which include, without limitation nilutamide (Nilandron®) and bicalutamide (Caxodex®).

antiestrogens which include, without limitation tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®, Keoxifene®) and raloxifene hydrochloride.

anti-hypercalcaemia agents which include without limitation gallium (III) nitrate hydrate (Ganite®) and pamidronate disodium (Aredia®).

apoptosis inducers which include without limitation ethanol, 2-[[3-(2,3-dichlorophenoxy)propyl]amino]-(9Cl), gambogic acid, elesclomol, embelin and arsenic trioxide (Trisenox®).

Aurora kinase inhibitors which include without limitation binucleine 2.

Bruton's tyrosine kinase inhibitors which include without limitation terreic acid.

calcineurin inhibitors which include without limitation cypermethrin, deltamethrin, fenvalerate and tyrphostin 8.

CaM kinase II inhibitors which include without limitation 5-Isoquinolinesulfonic acid, 4-[{2S)-2-[(5-isoquinolinylsulfonyl)methylamino]-3-oxo-3-{4-phenyl-1-piperazinyl)propyl]phenyl ester and benzenesulfonamide.

CD45 tyrosine phosphatase inhibitors which include without limitation phosphonic acid.

CDC25 phosphatase inhibitors which include without limitation 1,4-naphthalene dione, 2,3-bis[(2-hydroxyethyl)thio]-(9Cl).

CHK kinase inhibitors which include without limitation debromohymenialdisine.

cyclooxygenase inhibitors which include without limitation 1H-indole-3-acetamide, 1-(4-chlorobenzoyl)-5-methoxy-2-methyl-N-(2-phenylethyl)-(9Cl), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid).

cRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9Cl).

cyclin dependent kinase inhibitors which include without limitation olomoucine and its derivatives, purvalanol B, roascovitine (Seliciclib®), indirubin, kenpaullone, purvalanol A and indirubin-3′-monooxime.

cysteine protease inhibitors which include without limitation 4-morpholinecarboxamide, N-[(1S)-3-fluoro-2-oxo-1-(2-phenylethyl)propyl]amino]-2-oxo-1-(phenylmeth-yl)ethyl]-(9Cl).

DNA intercalators which include without limitation plicamycin (Mithracin®) and daptomycin (Cubicin®).

DNA strand breakers which include without limitation bleomycin (Blenoxane®).

E3 ligase inhibitors which include without limitation N-((3,3,3-trifluoro-2-trifluoromethyl)propionyl)sulfanilamide.

EGF Pathway Inhibitors which include, without limitation tyrphostin 46, EKB-569, erlotinib (Tarceva®), gefitinib (Iressa®), lapatinib (Tykerb®) and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980.

farnesyltransferase inhibitors which include without limitation a-hydroxyfarnesylphosphonic acid, butanoic acid, 2-[(2S)-2-[[(2S,3S)-2-[[(2R)-2-amino-3-mercaptopropyl]amino]-3-methylpent-yl]oxy]-1-oxo-3-phenylpropyl]amino]-4-(methylsulfonyl)-1-methylethylester (2S)-(9Cl), tipifarnib (Zarnestra®), and manumycin A.

Flk-1 kinase inhibitors which include without limitation 2-propenamide, 2-cyano-3-[4-hydroxy-3,5-bis(1-methylethyl)phenyl]-N-(3-phenylpropyl)-(2E-)-(9Cl).

glycogen synthase kinase-3 (GSK3) inhibitors which include without limitation indirubin-3′-monooxime.

histone deacetylase (HDAC) inhibitors which include without limitation suberoylanilide hydroxamic acid (SAHA), [4-(2-amino-phenylcarbamoyl)-benzyl]-carbamic acid pyridine-3-ylmethylester and its derivatives, butyric acid, pyroxamide, trichostatin A, oxamflatin, apicidin, depsipeptide, depudecin, largazole and related peptides, trapoxin, vorinostat (Zolinza®), and compounds disclosed in WO 02/22577.

I-kappa B-alpha kinase inhibitors (IKK) which include without limitation 2-propenenitrile, 3-[(4-methylphenyl)sulfonyl]-(2E)-(9Cl).

imidazotetrazinones which include without limitation temozolomide (Methazolastone®, Temodar® and its derivatives (e.g., as disclosed generically and specifically in U.S. Pat. No. 5,260,291) and Mitozolomide.

insulin tyrosine kinase inhibitors which include without limitation hydroxyl-2-naphthalenylmethylphosphonic acid.

c-Jun-N-terminal kinase (JNK) inhibitors which include without limitation pyrazoleanthrone and epigallocatechin gallate.

mitogen-activated protein kinase (MAP) inhibitors which include without limitation benzenesulfonamide, N-[2-[[[3-(4-chlorophenyl)-2-propenyl]methyl]amino]methyl]phenyl]-N-(2-hy-droxyethyl)-4-methoxy-(9Cl).

MDM2 inhibitors which include without limitation trans-4-iodo, 4′-boranyl-chalcone.

MEK inhibitors which include without limitation butanedinitrile, bis[amino[2-aminophenyl)thio]methylene]-(9Cl).

MMP inhibitors which include without limitation Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MMI270B or AAJ996.

mTor inhibitors which include without limitation rapamycin (Rapamune®), and analogs and derivatives thereof, AP23573 (also known as ridaforolimus, deforolimus, or MK-8669), CCI-779 (also known as temsirolimus) (Torisel®) and SDZ-RAD.

NGFR tyrosine kinase inhibitors which include without limitation tyrphostin AG 879.

p38 MAP kinase inhibitors which include without limitation Phenol, 4-[4-(4-fluorophenyl)-5-(4-pyridinyl)-1H-imidazol-2-yl]-(9Cl), and benzamide, 3-(dimethylamino)-N-[3-[(4-hydroxylbenzoyl)amino]-4-methylphenyl]-(9Cl).

p56 tyrosine kinase inhibitors which include without limitation damnacanthal and tyrphostin 46.

PDGF pathway inhibitors which include without limitation tyrphostin AG 1296, tyrphostin 9, 1,3-butadiene-1,1,3-tricarbonitrile, 2-amino-4-(1H-indol-5-yl)-(9Cl), imatinib (Gleevec®) and gefitinib (Iressa®) and those compounds generically and specifically disclosed in European Patent No.: 0 564 409 and PCT Publication No.: WO 99/03854.

phosphatidylinositol 3-kinase inhibitors which include without limitation wortmannin, and quercetin dihydrate.

phosphatase inhibitors which include without limitation cantharidic acid, cantharidin, and L-leucinamide.

protein phosphatase inhibitors which include without limitation cantharidic acid, cantharidin, L-P-bromotetramisole oxalate, 2(5H)-furanone, 4-hydroxy-5-(hydroxymethyl)-3-(1-oxohexadecyl)-(5R)-(9Cl) and benzylphosphonic acid.

PKC inhibitors which include without limitation 1-H-pyrollo-2,5-dione, 3-[1-3-(dimethylamino)propyl]-1H-indol-3-yl]-4-(1H-indol-3-yl)-(9Cl), Bisindolylmaleimide IX, Sphinogosine, staurosporine, and Hypericin.

PKC delta kinase inhibitors which include without limitation rottlerin.

polyamine synthesis inhibitors which include without limitation DMFO.

PTP1B inhibitors which include without limitation L-leucinamide.

protein tyrosine kinase inhibitors which include, without limitation tyrphostin Ag 216, tyrphostin Ag 1288, tyrphostin Ag 1295, geldanamycin, genistein and 7H-pyrrolo[2,3-d]pyrimidine derivatives as generically and specifically described in PCT Publication No.: WO 03/013541 and U.S. Publication No.: 2008/0139587.

SRC family tyrosine kinase inhibitors which include without limitation PP1 and PP2.

Syk tyrosine kinase inhibitors which include without limitation piceatannol.

Janus (JAK-2 and/or JAK-3) tyrosine kinase inhibitors which include without limitation tyrphostin AG 490 and 2-naphthyl vinyl ketone.

retinoids which include without limitation isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®).

RNA polymerase II elongation inhibitors which include without limitation 5,6-dichloro-1-beta-D-ribofuranosylbenzimidazole.

serine/Threonine kinase inhibitors which include without limitation 2-aminopurine.

sterol biosynthesis inhibitors which include without limitation squalene epoxidase and CYP2D6.

VEGF pathway inhibitors, which include without limitation anti-VEGF antibodies, e.g., bevacizumab, and small molecules, e.g., sunitinib (Sutent®), sorafinib (Nexavar®), ZD6474 (also known as vandetanib) (Zactima™), SU6668, CP-547632 and AZD2171 (also known as cediranib) (Recentin™).

Examples of chemotherapeutic agents are also described in the scientific and patent literature, see, e.g., Bulinski (1997) J. Cell Sci. 110:3055-3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem. 271:29807-29812.

Other exemplary anti-cancer agents include alitretinon, altretamine, aminopterin, aminolevulinic acid, amsacrine (Amsidine®), asparaginase (crisantaspase, Erwinase®), atrasentan, bexarotene (Targretin®), carboquone, demecolcine, efaproxiral, elsamitrucin, etoglucid, hydroxycarbamide, leucovorin, lonidamine, lucanthone, masoprocol, methyl aminolevulinate, mitoguazone, mitotane (Lysodren®), oblimersen, omacetaxine (Genasense®), pegaspargase (Oncaspar®), porfimer sodium (Photofrin®), prednimustine, sitimagene ceradenovec (Cerepro®), talaporfin, temoporfin, trabectedin (Yondelis®), and verteporfin.

### Administration

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

In some embodiments, co-administration of an effective amount of a compound of any of formulae (I)-(XVI) may be used in combination and optionally with other known breast cancer therapies, including surgery, anti-cancer agents (including hormonal and chemotherapeutic), immunotherapy, and radiation. Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the overall treatment is more effective because of combined administration. In some embodiments, delivery is such that the reduction in a symptom, or other parameter related to the disorder is greater than what would be observed with one treatment delivered in the absence of the other. The effect of the two treatments can be partially additive, wholly additive, or greater than additive (synergistic).

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human.

### Formulations

While it is possible for the active compound to be administered alone, it can be formulated as a pharmaceutical composition (e.g. formulation) comprising at least one active compound, as defined above, together with one or more pharmaceutically acceptable carriers, adjuvants, excipients, diluents, fillers, buffers, stabilizers, preservatives, lubricants, or other materials well known to those skilled in the art and optionally other therapeutic or prophylactic agents.

Thus, the disclosure further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, losenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include lozenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active compound.

Formulations suitable for administration by inhalation include those presented as an aerosol spray from a pressurized pack, with the use of a suitable propellant, such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Suitable emulgents and emulsion stabilizers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as diisoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g. by injection, including cutaneous, subcutaneous, intramuscular, intravenous and intradermal), include aqueous and nonaqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

### Dosages

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

### Examples

### General Considerations

Unless stated to the contrary, where applicable, the following conditions apply. Air sensitive reactions were carried out using dried solvents (see below) and under a slight static pressure of Ar (pre-purified quality) that had been passed through a column of Drierite. Glassware was dried in an oven at 120° C. for at least 12 h prior to use and then assembled quickly while hot, sealed with rubber septa, and allowed to cool under a stream of Ar. Reactions were stirred magnetically using Teflon-coated magnetic stirring bars. Teflon-coated magnetic stirring bars and syringe needles were dried in an oven at 120° C. for at least 12 h prior to use. Commercially available Norm-Ject disposable syringes were used. All ¹H and ¹³C NMR spectra were recorded on 300 MHz or 400 MHz Varian Mercury spectrometers as noted. ¹H spectra were referenced to CHCl₃ at 7.26 ppm and ¹³C spectra were referenced to CDCl₃ at 77.23 ppm. All spectra were taken in CDCl₃ unless otherwise noted. Thin layer chromatography (TLC) was carried out on Merck silica gel 60 F₂₅₄ aluminum backed plates and visualized using 254 nm UV light. Flash chromatographic purifications were performed using silica gel (40-60 μm) purchased from Agela Technologies (Newark, Del.). Compounds and solvents were obtained from Fisher, Sigma-Aldrich, and VWR and used without further purification. PCR reagents were obtained from Bio-Rad. PCR oligos were purchased from Integrated DNA Technologies and Sigma-Aldrich. siRNA were obtained from Invitrogen.

### Example 1

### Synthesis of Ethers

The following example is representative for the formation of all diaryl ethers from their respective phenols or thiophenols and para-bromobenzaldehyde.

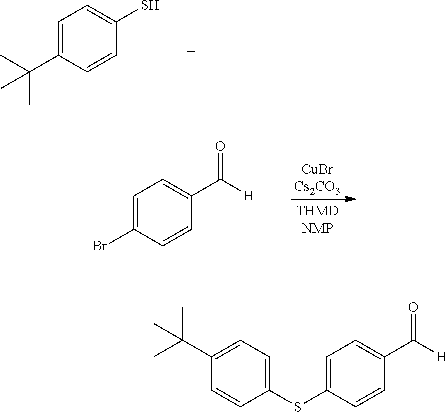 [SMILES: CC(C)(C)c1ccc(S)cc1 ; O=Cc1ccc(Br)cc1 ; CC(C)(C)c1ccc(Sc2ccc(C=O)cc2)cc1]

4-(4-tert-butylphenylthio)benzaldehyde: Under argon, an oven dried round bottom flask was charged with the 4-tert-butylthiophenol (0.69 mL, 4 mmol, 2 eq) and anhydrous N-methyl-2-pyrrolidone (5 mL). Cesium carbonate (1.3 g, 4 mmol, 2 eq) was added to the stirring solution and it immediately turned cloudy. Para-bromobenzaldehyde (370 mg, 2 mmol, 1 eq) was added, followed by copper (I) bromide (143 mg, 1 mmol, 0.5 eq) and 2,2,6,6-tetramethyl-3,5-heptanedione (41 μL, 0.2 mmol, 0.1 eq). The flask was equipped with a reflux condenser and heated to 70-80° C. for 15.5 h while stirring. After cooling to rt, the reaction mixture was diluted with methyl tert-butyl ether (100 mL) and vacuum filtered. The residue was washed with MTBE (100 mL) and the combined filtrates were washed with 2 N HCl (100 mL), 0.6 N HCl (100 mL), 2 M NaOH (100 mL), and saturated NaCl (100 mL). The organic layer was dried over MgSO₄, filtered, and concentrated in vacuo. The desired 4-(4-tert-butylphenylthio)benzaldehyde was isolated by flash chromatography over silica gel with 10:1 hexanes:ethyl acetate to afford a gold oil in 62% yield (0.065 g). ¹H NMR (400 MHz, CDCl₃): δ9.88 (1H, s), 7.69 (2H, d, J=8.4 Hz), 7.44 (4H, m), 7.20 (2H, d, J=8.4 Hz), 1.34 (9H, s). ¹³C NMR (100 MHz, CDCl₃): δ191.2, 152.7, 148.0, 134.4, 133.5, 130.1, 127.3, 126.9, 126.7, 34.8, 31.2.

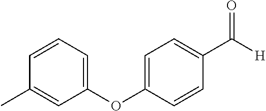 [SMILES: Cc1cccc(Oc2ccc(C=O)cc2)c1]

4-(m-tolyloxy)benzaldehyde: 0.049 g, 54%, white solid. ¹H NMR (300 MHz, CDCl₃): δ9.92 (1H, s), 7.83 (2H, m), 7.27 (1H, m), 7.03 (3H, m), 6.90 (2H, m), 2.36 (3H, s). ¹³C NMR (75 MHz, CDCl₃): δ191.0, 132.2, 130.1, 126.0, 121.3, 117.8, 117.6, 21.6.

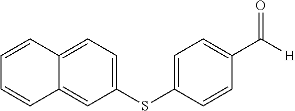 [SMILES: O=Cc1ccc(Sc2ccc3ccccc3c2)cc1]

4-(naphthalen-2-ylthio)benzaldehyde: 0.071 g, 60%, off-white solid. ¹H NMR (400 MHz, CDCl₃): δ9.90 (1H, s), 8.07 (1H, s), 7.82 (3H, m), 7.70 (2H, m), 7.54 (3H, m), 7.27 (2H, m). ¹³C NMR (100 MHz, CDCl₃): δ191.2, 147.1, 134.0, 133.8, 133.7, 133.1, 130.7, 130.2, 129.6, 127.9, 127.8, 127.3, 127.2, 126.9.

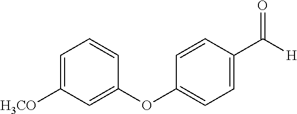 [SMILES: COc1cccc(Oc2ccc(C=O)cc2)c1]

4-(3-methoxyphenoxy)benzaldehyde: 0.204 g, 44%, yellow oil. ¹H NMR (300 MHz, CDCl₃): δ9.89 (1H, s), 7.83 (2H, m), 7.28 (1H, m), 7.07 (2H, m), 6.77 (1H, m), 6.66 (2H, m), 3.77 (3H, s). ¹³C NMR (75 MHz, CDCl₃): δ190.6, 162.8, 161.0, 156.0, 131.8, 131.2, 130.4, 117.5, 112.2, 110.4, 106.2, 55.2.

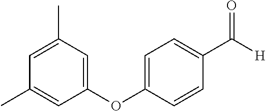 [SMILES: Cc1cc(C)cc(Oc2ccc(C=O)cc2)c1]

4-(3,5-dimethylphenoxy)benzaldehyde: 0.176 g, 38%, gold oil. ¹H NMR (300 MHz, CDCl₃): δ9.91 (1H, s), 7.82 (2H, m), 7.03 (2H, m), 6.85 (1H, s), 6.70 (2H, s), 2.31 (6H, s). ¹³C NMR (75 MHz, CDCl₃): δ191.0, 163.7, 155.2, 140.3, 132.2, 131.3, 126.9, 118.3, 117.7, 21.5.

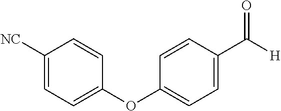 [SMILES: N#Cc1ccc(Oc2ccc(C=O)cc2)cc1]

4-(4-formylphenoxy)benzonitrile: 0.082 g, 18%, gold solid. ¹H NMR (400 MHz, CDCl₃): δ9.98 (1H, s), 7.92 (2H, m), 7.68 (2H, m), 7.15 (4H, m). ¹³C NMR (100 MHz, CDCl₃): δ190.9, 159.8, 134.7, 133.0, 132.4, 128.8, 119.9, 119.7, 118.6, 107.9.

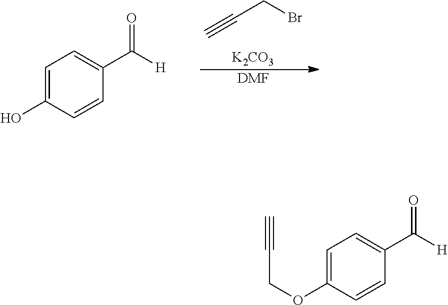

4-(prop-2-ynyloxy)benzaldehyde: An oven-dried round bottom flask was charged with anhydrous dimethylformamide (35 mL), 4-hydroxybenzaldehyde (2.08 g, 16.4 mmol, 1 eq) and anhydrous potassium carbonate (6.80 g, 49.2 mmol, 3 eq) and was stirred at 55° C. for 30 min. The reaction mixture was cooled to rt and propargyl bromide (1.75 mL, 19.7 mmol, 1.2 eq) was added. The reaction was stirred for an additional 5 h at rt. The crude reaction mixture was poured on ice water (100 mL) and stirred for 10 min. The desired 4-(prop-2-ynyloxybenzaldehyde) was isolated by vacuum filtration and dried in vacuo over CaSO₄ to afford a brown solid in 92% yield (2.520 g). ¹H NMR (300 MHz, CDCl₃): δ9.90 (1H, s), 7.86 (2H, m), 7.10 (2H, m), 4.79 (2H, d, J=2.7 Hz), 2.57 (1H, J=2.4 Hz). ¹³C NMR (75 MHz, CDCl₃): δ191.0, 132.1, 115.4, 76.6, 56.2.

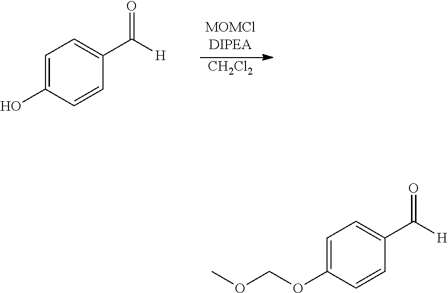

4-(methoxymethoxy)benzaldehyde: Diisopropylethylamine (7.1 mL, 41 mmol, 2.5 eq) was added slowly dropwise to a stirring solution of 4-hydroxybenzaldehyde (2.0 g, 16.4 mmol, 1 eq) in dichloromethane (50 mL). Chloromethyl methyl ether (1.9 mL, 24.6 mmol, 1.5 eq) was added slowly producing a gas. The reaction was stirred at rt for 1.25 h. The reaction was quenched by adding water (50 mL). The organic products were extracted with ethyl acetate (3×25 mL), dried with anhydrous Na₂SO₄ and concentrated to a golden oil under reduced pressure.

The desired 4-(methoxymethoxy)benzaldehyde was isolated by flash chromatography over silica gel with 4:1 hexanes:ethyl acetate to afford a clear colorless oil in 83% yield (2.227 g). ¹H NMR (400 MHz, CDCl₃): δ9.87 (1H, s), 7.82 (1H, d, J=8.8 Hz), 7.14 (2H, d, J=8.8 Hz), 5.24 (2H, s), 3.47 (3H, s). ¹³C NMR (100 MHz, CDCl₃): δ191.0, 162.3, 131.9, 130.8, 116.4, 56.4.

### Example 2

### Synthesis of Methyl Esters from Cinnamic Acids

The following example is representative for the formation of all methyl esters from their respective cinnamic acids with acidic methanol.

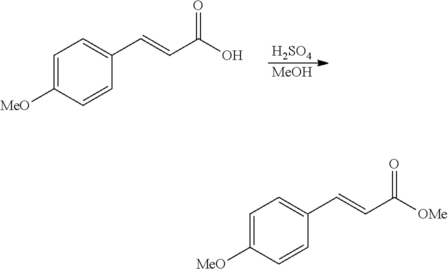

(E)-methyl 3-(4-methoxyphenyl)acrylate: In a round bottom flask, 4-methoxycinnamic acid (3.14 g, 17 mmol, 1 eq) was suspended in methanol (20 mL). Sulfuric acid (650 μL) was added dropwise. The reaction was brought to 70° C. and allowed to reflux for 3.5 hours, until starting material was consumed as observed by TLC. The crude reaction mixture was poured on ice water (30 mL). The organic products were extracted with ether (1×60 mL, 2×30 mL), washed with brine (30 mL) and dried using anhydrous MgSO₄. The crude product was concentrated in vacuo. The desired (E)-methyl 3-(4-methoxyphenyl)acrylate was isolated by flash chromatography using 5:1 hexanes:ethyl acetate to afford a white solid in 96% yield (3.241 g). ¹H NMR (300 MHz, CDCl₃): δ7.61 (1H, d, J=15.9 Hz), 7.41 (2H, d, J=8.7 Hz), 6.84 (2H, d, J=8.7 Hz), 6.27 (1H, d, J=15.9 Hz), 3.83 (3H, s), 3.79 (3H, s). ¹³C NMR (75 MHz, CDCl₃): δ167.8, 161.6, 144.6, 129.9, 127.2, 115.3, 114.4, 55.4, 51.6.

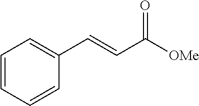 [SMILES: COC(=O)/C=C/c1ccccc1]

Methyl cinnamate: 7.947 g, 73%, white solid. ¹H NMR (400 MHz, CDCl₃): δ7.70 (1H, d, J=16 Hz), 7.51 (2H, m), 7.37 (2H, m), 6.46 (1H, d, J=1.8 Hz), 6.42 (1H, d, J=1.8 Hz), 3.81 (3H, s). ¹³C NMR (100 MHz, CDCl₃): δ145.1, 134.6, 130.5, 129.1, 128.3, 118.1, 51.9.

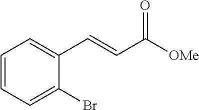 [SMILES: COC(=O)/C=C/c1ccccc1Br]

(E)-methyl 3-(2-bromophenyl)acrylate: 0.094 g, 89%, light gold oil. ¹H NMR (300 MHz, CDCl₃): δ8.05 (1H, d, J=15.9 Hz), 7.58 (2H, m), 7.29 (2H, m), 6.39 (1H, d, J=15.9 Hz), 3.82 (3H, s).

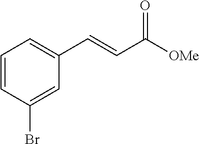 [SMILES: COC(=O)/C=C/c1cccc(Br)c1]

(E)-methyl 3-(3-bromophenyl)acrylate: 1.622 g, 77%, white solid. ¹H NMR (300 MHz, CDCl₃): δ 7.50 (1H, d, J=16.2 Hz), 7.44 (1H, s), 7.36 (1H, d, J=8.1 Hz), 7.28 (1H, d, J=8.1 Hz), 7.10 (1H, t, J=7.8 Hz), 6.30 (1H, J=16.2 Hz), 3.70 (3H, s). ¹³C NMR (100 MHz, CDCl₃): δ167.0, 143.2, 136.6, 133.2, 130.9, 130.5, 126.8, 123.2, 119.4.

### Example 3

### Synthesis of Methyl Esters from Cinnamic Acids with TMSCHN₂

The following example is representative for the formation of all methyl esters from their respective cinnamic acids with TMSCHN₂.

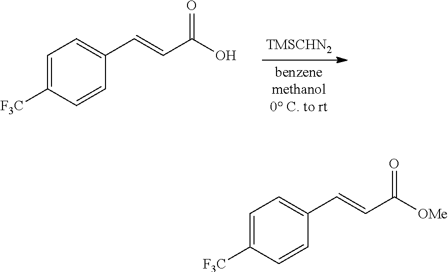 [SMILES: O=C(O)/C=C/c1ccc(C(F)(F)F)cc1 ; COC(=O)/C=C/c1ccc(C(F)(F)F)cc1]

(E)-methyl 3-(4-trifluoromethyl)phenyl)acrylate: A solution of TMSCHN₂ (2.0 M in hexanes, 1.6 eq) was added dropwise with stirring to a 0° C. solution of the (E)-3-(4-(trifluoromethyl)phenyl)acrylic acid (0.25 M) in benzene:methanol (2:1). The reaction was allowed to warm to rt over the course of 0.5 h. Concentration of the reaction mixture afforded the desired (E)-methyl 3-(4-trifluoromethyl)phenyl)acrylate as a white solid in 99% yield (0.898 g). ¹H NMR (300 MHz, CDCl₃): δ7.69 (1H, d, J=15.9 Hz), 7.62 (4H, m), 6.50 (1H, d, J=15.9 Hz), 3.87 (3H, s). ¹³C NMR (75 MHz, CDCl₃): δ167.0, 143.2, 138.0, 128.4, 126.1, 126.0, 120.6, 52.1.

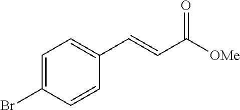 [SMILES: COC(=O)/C=C/c1ccc(Br)cc1]

(E)-methyl 3-(4-bromophenyl)acrylate: 1.47 g, 99%, off-white solid. ¹H NMR (300 MHz, CDCl₃): δ7.62 (1H, d, J=15.9 Hz), 7.51 (2H, d, J=8.1 Hz), 7.37 (2H, d, J=8.1 Hz), 6.42 (1H, d, J=15.9 Hz), 3.80 (3H, s). ¹³C NMR (75 MHz, CDCl₃): δ167.4, 143.7, 133.5, 132.4, 129.7, 124.8, 118.7, 52.0.

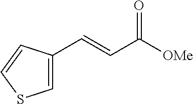 [SMILES: COC(=O)/C=C/c1ccsc1]

(E)-methyl 3-(thiophen-3-yl)acrylate: 1.22 g, 99%, light brown solid. ¹H NMR (300 MHz, CDCl₃): δ7.67 (1H, d, J=15.6 Hz), 7.49 (1H, m), 7.29 (1H, m), 7.33 (1H, m), 6.26 (1H, d, J=15.6 Hz), 3.79 (3H, s). ¹³C NMR (75 MHz, CDCl₃): δ167.9, 138.6, 137.8, 128.3, 127.2, 125.4, 117.7, 51.9.

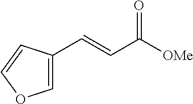 [SMILES: COC(=O)/C=C/c1ccoc1]

(E)-methyl 3-(furan-3-yl)acrylate: 1.11 g, 98%, off-white solid. ¹H NMR (300 MHz, CDCl₃): δ7.63 (1H,$), 7.56 (1H, d, J=15.6 Hz), 7.41 (1H, s), 6.57 (1H, s), 6.14 (1H, d, J=15.6 Hz), 3.76 (3H, s). ¹³C NMR (75 MHz, CDCl₃): δ167.6, 144.7, 144.6, 135.0, 122.8, 117.8, 107.6, 51.8.

### Example 4

### Synthesis of Trans-Alkenes Via Horner-Wadsworth-Emmons Olefination

The following example is representative for the formation of all trans-alkenes from the corresponding benzaldehyde and methyl diethylphosphonoacetate through a Horner-Wadsworth-Emmons olefination.

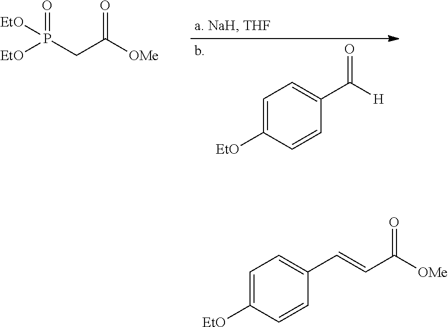 [SMILES: CCOP(=O)(CC(=O)OC)OCC ; CCOc1ccc(C=O)cc1 ; CCOc1ccc(/C=C/C(=O)OC)cc1]

(E)-methyl 3-(4-ethoxyphenyl)acrylate: Under argon, sodium hydride (252 mg, 10 mmol, 1.7 eq) was dissolved in anhydrous THF (40 mL) in an oven dried round bottom flask and cooled to 0° C. Methyl diethyl phosphonoacetate (1.7 mL, 9.6 mmol, 1.6 eq) was added dropwise and stirred for 45 min while allowing to warm to rt. In round bottom flask under argon, para-ethoxybenzaldehyde (830 μL, 6 mmol, 1.0 eq) was dissolved in anhydrous toluene (60 mL) and cooled to −78° C. The phosphonate anion solution was transferred to the aldehyde via cannula and the reaction was allowed to warm to rt over the course of 6.75 h. Saturated Rochelle's salt (20 mL) was added and stirred for 10 min. CH₂Cl₂ (20 mL) and deionized water (20 mL) was added and the layers separated. The organic products were extracted with CH₂Cl_{2 (}3×30 mL), dried over anhydrous Na₂SO₄ and the solvent was removed under reduced pressure. The desired (E)-methyl 3-(4-ethoxyphenyl)acrylate was isolated by flash chromatography using 100% CH₂Cl₂ to yield a white solid in 95% yield (1.164 g). ¹H NMR (300 MHz, CDCl₃): δ7.64 (1H, d, J=15.9 Hz), 7.46 (2H, d, J=8.7 Hz), 6.88 (2H, d, J=8.7 Hz), 6.30 (1H, d, J=15.9 Hz), 4.05 (2H, q, J=6.9 Hz), 4.79 (3H, s), 1.42 (3H, t, J=6.9 Hz). ¹³C NMR (75 MHz, CDCl₃): δ168.0, 161.0, 144.8, 129.9, 127.2, 115.3, 115.1, 63.8, 51.8, 14.9.

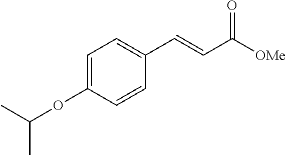 [SMILES: COC(=O)/C=C/c1ccc(OC(C)C)cc1]

(E)-methyl 3-(4-isopropoxyphenyl)acrylate: 0.651 g, 95%, clear, colorless oil. ¹H NMR (300 MHz, CDCl₃): δ7.62 (1H, d, J=15.9 Hz), 7.37 (2H, m), 6.91 (2H, m), 6.20 (1H, d, J=15.9 Hz), 3.74 (3H, s), 2.96 (6H, s). ¹³C NMR (75 MHz, CDCl₃): δ168.5, 152.0, 145.6, 130.0, 122.3, 112.2, 112.0, 51.6, 40.3.

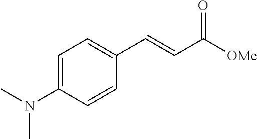

(E)-methyl 3-(4-(dimethylamino)phenyl)acrylate: 0.456 g, 86%; light yellow solid. ¹H NMR (300 MHz, CDCl₃): δ7.62 (1H, d, J=15.9 Hz), 7.37 (2H, m), 6.91 (2H, m), 6.20 (1H, d, J=15.9 Hz), 3.74 (3H, s), 2.96 (6H, s). ¹³C NMR (75 MHz, CDCl₃): δ168.5, 152.0, 145.6, 130.0, 122.3, 112.2, 112.0, 51.6, 40.3.

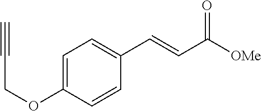 [SMILES: C#CCOc1ccc(/C=C/C(=O)OC)cc1]

(E)-methyl 3-(4-(prop-2-ynyloxy)phenyl)acrylate: 1.359 g, 87%, white solid. ¹H NMR (400 MHz, CDCl₃): δ7.48 (1H, d, J=16 Hz), 7.32 (2H, d, J=8.8 Hz), 6.82 (2H, d, 8.8 Hz), 6.17 (1H, d, J=16 Hz), 4.56 (2H, d, J=2.4 Hz), 3.62 (3H, s), 2.57 (1H, t, J=2.4 Hz).

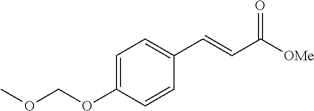 [SMILES: COCOc1ccc(/C=C/C(=O)OC)cc1]

(E)-methyl 3-(4-(methoxymethoxy)phenyl)acrylate: 1.362 g, 98%, white solid. ¹H NMR (400 MHz, CDCl₃): δ7.64 (1H, d, J=16 Hz), 7.44 (2H, m), 7.02 (2H, m), 6.32 (1H, d, J=16 Hz), 5.19 (2H, s), 3.75 (3H, s), 3.47 (3H, s). ¹³C NMR (100 MHz, CDCl₃): δ167.8, 159.1, 144.6, 129.8, 128.3, 116.6, 116.0, 94.3, 56.3, 51.7.

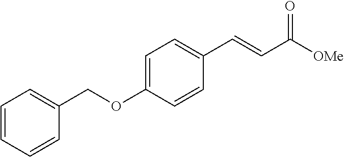 [SMILES: COC(=O)/C=C/c1ccc(OCc2ccccc2)cc1]

(E)-methyl 3-(4-(benzyloxy)phenyl)acrylate: 1.120 g, 83%, white solid. ¹H NMR (300 MHz, CDCl₃): δ7.64 (1H, d, J=16 Hz), 7.41 (7H, m), 6.96 (2H, d, J=8.7 Hz), 6.30 (1H, d, J=16 Hz), 5.08 (2H, s), 3.78 (3H, s). ¹³C NMR (75 MHz, CDCl₃): δ144.4, 129.7, 128.6, 128.1, 127.4, 115.3, 115.1, 70.0, 51.5.

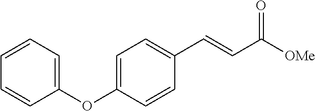 [SMILES: COC(=O)/C=C/c1ccc(Oc2ccccc2)cc1]

(E)-methyl 3-(4-phenoxyphenyl)acrylate: 0.395 g, 67%, white solid. ¹H NMR (300 MHz, CDCl₃): δ7.65 (1H, d, J=15.9 Hz), 7.45 (2H, m), 7.32 (2H, m), 7.14 (1H, m), 7.00 (4H, m), 6.33 (1H, d, J=15.9 Hz), 3.77 (3H, s). ¹³C NMR (75 MHz, CDCl₃): δ167.7, 159.7, 156.3, 144.3, 130.2, 130.0, 129.4, 124.4, 119.9, 118.6, 116.7, 51.9.

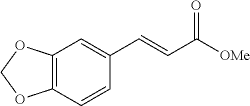 [SMILES: COC(=O)/C=C/c1ccc2c(c1)OCO2]

(E)-methyl 3-(benzo[d][1,3]dioxol-5-yl)acrylate: 1.097 g, 89%, white solid. ¹H NMR (300 MHz, CDCl₃): δ7.28 (1H, d, J=15.9 Hz), 7.00 (2H, m), 6.79 (2H, m), 6.25 (1H, d, J=15.9 Hz), 3.78 (3H, s). ¹³C NMR (75 MHz, CDCl₃): δ167.6, 149.6, 148.3, 144.5, 128.8, 124.4, 115.7, 108.5, 106.5, 101.5, 51.6.

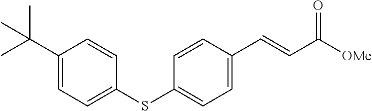 [SMILES: COC(=O)/C=C/c1ccc(Sc2ccc(C(C)(C)C)cc2)cc1]

(E)-methyl 3-(4-(4-tert-butylphenylthio)phenyl)acrylate: 0.298 g, 76%, white solid. ¹H NMR (300 MHz, CDCl₃): δ7.63 (1H, d, J=15.9), 7.38 (6H, m), 7.19 (2H, m), 6.37 (1H, d, J=15.9 Hz), 3.79 (3H, s), 1.33 (9H, s). ¹³C NMR (75 MHz, CDCl₃): δ167.3, 151.6, 144.0, 141.1, 133.1, 131.8, 129.2, 128.4, 128.4, 126.5, 117.0, 51.6, 34.6, 31.1.

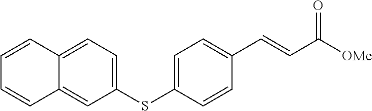 [SMILES: COC(=O)/C=C/c1ccc(Sc2ccc3ccccc3c2)cc1]

(E)-methyl 3-(4-(naphthalen-2-ylthio)phenyl)acrylate: 0.314 g, 100%, white solid. ¹H NMR (300 MHz, CDCl₃): δ7.97 (1H, s), 7.78 (3H, m), 7.63 (1H, d, J=16.2 Hz), 7.48 (5H, m), 7.25 (2H, m), 6.39 (1H, d, J=16.2 Hz). ¹³C NMR (75 MHz, CDCl₃): δ144.3, 132.3, 130.0, 129.5, 128.9, 128.0, 127.9, 127.0, 117.7, 52.0.

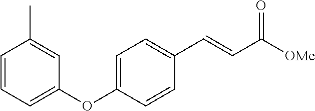 [SMILES: COC(=O)/C=C/c1ccc(Oc2cccc(C)c2)cc1]

(E)-methyl 3-(4-(m-tolyloxy)phenyl)acrylate (2.30): 67%, white solid. ¹H NMR (300 MHz, CDCl₃): δ7.60 (1H, d, J=12 Hz), 7.39 (2H, m), 7.26 (1H, m), 7.16 (1H, m), 6.89 (2H, m), 6.78 (2H, m), 6.30 (1H, m), 3.72 (3H, s).

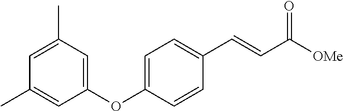 [SMILES: COC(=O)/C=C/c1ccc(Oc2cc(C)cc(C)c2)cc1]

(E)-methyl 3-(4-(3,5-dimethylphenoxy)phenyl)acrylate: 0.131 g, 95%, clear gold oil. ¹H NMR (300 MHz, CDCl₃): δ7.66 (1H, d, J=15.9 Hz), 7.46 (2H, m), 6.96 (2H, m), 6.80 (1H, s), 6.66 (2H, s), 6.34 (1H, d, J=15.9 Hz), 3.79 (3H, s), 2.29 (6H, s). ¹³C NMR (75 MHz, CDCl₃): δ167.8, 160.0, 156.2, 144.5, 140.1, 130.0, 129.1, 126.1, 118.5, 117.6, 116.5, 51.9, 21.5.

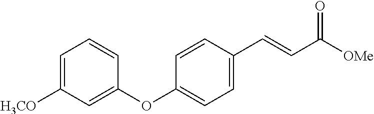 [SMILES: COC(=O)/C=C/c1ccc(Oc2cccc(OC)c2)cc1]

(E)-methyl 3-(4-(3-methoxyphenoxy)phenyl)acrylate: 0.213 g, 82%, clear gold oil. ¹H NMR (300 MHz, CDCl₃): δ7.65 (1H, d, J=15.9 Hz), 7.45 (2H, m), 7.23 (1H, m), 6.97 (2H, m), 6.69 (1H, m), 6.60 (2H, m), 6.34 (1H, d, J=15.9 Hz), 3.77 (3H, s), 3.76 (3H, s). ¹³C NMR (75 MHz, CDCl₃): δ167.8, 161.3, 159.5, 157.5, 144.3, 130.6, 130.0, 129.5, 118.8, 116.7, 111.9, 110.0, 105.9, 55.6, 51.9.

### Example 5

### Cyclopropanation of Acrylates Using Diazomethane

The following example is representative for the cyclopropanation of the acrylates using diazomethane.

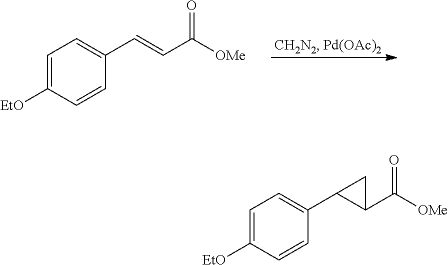 [SMILES: CCOc1ccc(/C=C/C(=O)OC)cc1 ; CCOc1ccc(C2CC2C(=O)OC)cc1]

Methyl trans-2-(4-ethoxyphenyl)cyclopropanecarboxylate: The diazomethane generator was used. The (E)-methyl 3-(4-ethoxyphenyl)acrylate (1.8 mmol, 1 eq) and palladium (II) acetate catalyst (8.0 mg, 1.6 mol %) were dissolved in diethyl ether (26 mL) in the round bottom flask. 85% Potassium hydroxide pellets (2.81 g, 42 mmol, 23 eq) was dissolved in water (10 mL) and diethylene glycol monoethyl ether (15 mL) in the distillation chamber and brought to 60-70° C. using an oil bath. The cold finger was brought to −72° C. using isopropyl alcohol/dry ice and the round bottom brought to <−25° C. using ethylene glycol/dry ice. Diazald (3.90 g, 18 mmol, 10 eq) dissolved in diethyl ether (30 mL) was added dropwise from the addition funnel to the distillation chamber. The produced diazomethane was distilled into the round bottom collecting the clear golden yellow liquid. The round bottom capped loosely and stirred overnight (12-20 h) allowing to warm to rt. The reaction mixture was run over a plug of celite to remove the catalyst and the solvent removed by in vacuo. The reaction was monitored by ¹H NMR, and if alkene was still present (1H d˜6.3 ppm, 1H d˜7.6 ppm), additional equivalents of reagents were added to drive reaction to completion. No other purification was necessary to yield the white solid in 99% yield (0.286 g). ¹H NMR (400 MHz, CDCl₃): δ6.99 (2H, m), 6.79 (2H, m), 3.97 (2H, q, J=7.6 Hz), 3.69 (3H, s), 2.48 (1H, ddd, J=4.2, 4.4, 11.3 Hz), 1.81 (2H, quintet, J=4.2), 1.54 (1H), 1.38 (3H, t, J=4.2), 1.25 (1H). ¹³C NMR (100 MHz, CDCl₃): δ174.2, 157.9, 132.0, 127.5, 114.7, 63.6, 52.0, 26.0, 23.8, 16.9, 15.0.

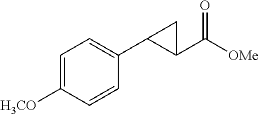 [SMILES: COC(=O)C1CC1c1ccc(OC)cc1]

Methyl trans-2-(4-methoxyphenyl)cyclopropanecarboxylate: 0.343 g, 91%, light yellow solid. ¹H NMR (300 MHz, CDCl₃): δ7.00 (2H, m), 6.78 (2H, m), 3.72 (3H, s), 3.70 (3H, s), 2.49 (1H, ddd, J=4.5, 6.6, 8.4 Hz), 1.83 (2H, quintet, J=4.5 Hz), 1.56 (1H, m), 1.27 (1H, m). ¹³C NMR (75 MHz, CDCl₃): δ174.0, 158.7, 132.1, 127.6, 114.2, 55.3, 51.9, 25.8, 23.8, 16.8.

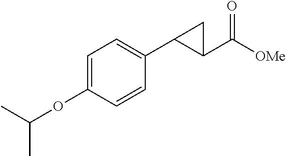 [SMILES: COC(=O)C1CC1c1ccc(OC(C)C)cc1]

Methyl trans-2-(4-isopropoxyphenyl)cyclopropanecarboxylate: 0.408 g, 100%, clear yellow oil. ¹H NMR (300 MHz, CDCl₃): δ6.98 (2H, m), 6.78 (2H, m), 4.47 (1H, sep, J=6 Hz), 2.47 (1H, ddd, J=4.3, 4.5, 11.3), 1.82 (1H, q, J=4.2 Hz), 1.54 (1H, q, J=4.2 Hz). ¹³C NMR (75 MHz, CDCl₃): δ174.1, 156.9, 131.9, 127.6, 116.2, 70.1, 51.9, 26.0, 23.9, 22.2, 16.9.

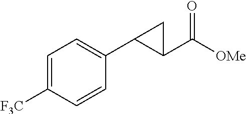 [SMILES: COC(=O)C1CC1c1ccc(C(F)(F)F)cc1]

Methyl trans-2-(4-(trifluoromethyl)phenyl)cyclopropanecarboxylate: 0.423 g, 96%, pale yellow solid. ¹H NMR (300 MHz, CDCl₃): δ7.51 (2H, d, J=8.1 Hz), 7.17 (2H, d, J=8.1 Hz), 3.71 (3H, s), 2.56 (1H, ddd, J=4.2, 6.3, 9.0 Hz), 1.94 (1H, ddd, J=4.2, 5.4, 8.7 Hz), 1.65 (1H, m), 1.33 (1H, ddd, J=4.8, 6.6, 8.4 Hz). ¹³C NMR (75 MHz, CDCl₃): δ173.6, 144.5, 125.6, 52.3, 26.0, 24.5, 17.5.

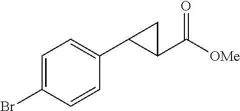 [SMILES: COC(=O)C1CC1c1ccc(Br)cc1]

Methyl trans-2-(4-bromophenyl)cyclopropanecarboxylate: 0.435 g, 94%, pale yellow oil. ¹H NMR (300 MHz, CDCl₃): δ7.39 (2H, d, J=8.4 Hz), 6.97 (2H, d, J=8.4 Hz), 3.72 (3H, s), 2.48 (1H, ddd, J=4.5, 6.6, 8.4 Hz), 1.87 (1H, ddd, J=4.2, 5.1, 8.4 Hz), 1.60 (1H, ddd, J=4.5, 5.4, 9.3 Hz), 1.28 (1H, ddd, J=4.8, 6.6, 8.4 Hz). ¹³C NMR (75 MHz, CDCl₃): δ173.8, 139.3, 131.8, 128.2, 120.4, 52.2, 25.2, 24.1, 17.2.

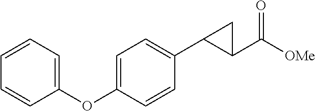 [SMILES: COC(=O)C1CC1c1ccc(Oc2ccccc2)cc1]

Methyl trans-2-(4-phenoxyphenyl)cyclopropanecarboxylate: 0.279 g, 100%, light gold oil. ¹H NMR (300 MHz, CDCl₃): δ7.28 (2H, m), 7.02 (3H, m), 6.94 (4H, m), 3.69 (3H, s), 2.51 (1H, ddd), 1.86 (1H, ddd), 1.58 (1H, ddd), 1.27 (1H, ddd). ¹³C NMR (75 MHz, CDCl₃): δ174.1, 157.6, 156.1, 135.1, 130.0, 127.9, 123.4, 119.3, 118.9, 52.1, 30.0, 26.0, 24.1, 17.1.

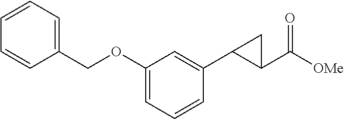 [SMILES: COC(=O)C1CC1c1cccc(OCc2ccccc2)c1]

Methyl trans-2-(3-(benzyloxy)phenyl)cyclopropanecarboxylate: 0.298 g, 100%, off-white solid. ¹H NMR (300 MHz, CDCl₃): δ 7.37 (4H, m), 7.05 (2H, m), 6.92 (2H, m), 5.04 (2H, s), 3.73 (3H, s), 2.53 (1H, ddd), 1.86 (1H, ddd), 1.59 (1H, ddd), 1.29 (1H, ddd). ¹³C NMR (75 MHz, CDCl₃): δ147.2, 157.8, 137.3, 132.5, 128.9, 128.2, 127.7, 115.2, 70.3, 52.1, 26.0, 24.0, 17.0.

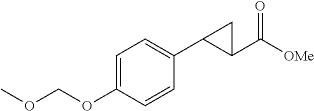 [SMILES: COCOc1ccc(C2CC2C(=O)OC)cc1]

Methyl trans-2-(4-(methoxymethoxy)phenyl)cyclopropanecarboxylate: 0.420 g, 100%, white solid. ¹H NMR (400 MHz, CDCl₃): δ7.00 (2H, m), 6.94 (2H, m), 5.11 (2H, s), 3.68 (3H, s), 3.43 (3H, s), 2.47 (1H), 1.82 (1H), 1.54 (1H), 1.25 (1H). ¹³C NMR (100 MHz, CDCl₃): δ174.0, 156.1, 133.4, 127.5, 116.5, 94.6, 56.0, 51.9, 25.8, 23.8, 16.8.

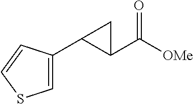 [SMILES: COC(=O)C1CC1c1ccsc1]

Methyl trans-2-(thiophen-3-yl)cyclopropanecarboxylate: 0.163 g, 99%, amber oil. ¹H NMR (300 MHz, CDCl₃): δ7.23 (1H, m), 6.95 (1H, m), 6.83 (1H, m), 3.71 (3H, s), 2.56 (1H, ddd, J=4.2, 6.6, 9.0 Hz), 1.87 (1H, ddd, J=4.2, 5.1, 8.4 Hz), 1.56 (1H, ddd, J=4.5, 5.1, 9.0 Hz), 1.26 (1H, ddd, J=4.5, 6.3, 8.4 Hz). ¹³C NMR (75 MHz, CDCl₃): δ173.9, 141.3, 126.2, 126.1, 120.0, 52.1, 23.6, 22.2, 17.1.

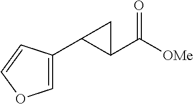 [SMILES: COC(=O)C1CC1c1ccoc1]

Methyl trans-2-(furan-3-yl)cyclopropanecarboxylate: 0.052 g, 97%, clear yellow oil. ¹H NMR (300 MHz, CDCl₃): δ7.32 (1H, m), 7.28 (1H, m), 6.15 (1H, m), 3.71 (3H, s), 2.33 (1H, ddd, J=4.2, 6.6, 9.0 Hz), 1.76 (1H, ddd, J=3.9, 5.1, 8.4 Hz), 1.50 (1H, m), 1.13 (1H, ddd, J=4.5, 6.6, 8.1 Hz). ¹³C NMR (75 MHz, CDCl₃): δ174.1, 143.4, 139.4, 124.9, 109.2, 52.1, 22.7, 17.5, 16.3.

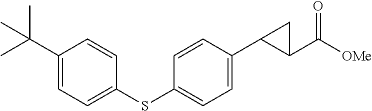 [SMILES: COC(=O)C1CC1c1ccc(Sc2ccc(C(C)(C)C)cc2)cc1]

Methyl trans-2-(4-(4-tert-butylphenylthio)phenyl)cyclopropanecarboxylate: 0.246 g, 94%, gold oil. ¹H NMR (300 MHz, CDCl₃): δ7.25 (5H, m), 6.99 (2H, m), 3.69 (3H, s), 2.41 (1H, ddd), 1.87 (1H, ddd), 1.59 (1H, ddd), 1.29 (10H, m). ¹³C NMR (100 MHz, CDCl₃): δ173.9, 150.6, 139.1, 134.5, 132.4, 131.2, 127.2, 126.5, 52.2, 34.8, 31.5, 26.2, 24.3, 17.3.

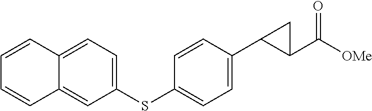 [SMILES: COC(=O)C1CC1c1ccc(Sc2ccc3ccccc3c2)cc1]

Methyl trans-2-(4-(naphthalen-2-ylthio)phenyl)cyclopropanecarboxylate: 0.254 g, 95%, gold oil. ¹H NMR (300 MHz, CDCl₃): δ7.70 (4H, m), 7.42 (5H, m), 6.99 (2H, m), 3.69 (3H, s), 2.49 (1H, ddd), 1.88 (1H, ddd), 1.60 (1H, ddd), 1.26 (1H, ddd). ¹³C NMR (75 MHz, CDCl₃): δ173.9, 139.7, 134.0, 133.7, 132.4, 131.9, 129.5, 129.1, 128.6, 128.0, 127.6, 127.4, 126.9, 126.4, 52.2, 26.2, 24.4, 17.4.

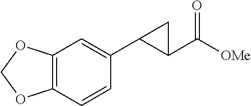 [SMILES: COC(=O)C1CC1c1ccc2c(c1)OCO2]

Methyl 2-(benzo[d][1,3]dioxol-5-yl)cyclopropanecarboxylate: 0.405 g, 100%, white solid. ¹H NMR (300 MHz, CDCl₃): δ6.68 (2H, m), 6.54 (2H, m), 5.88 (2H, s), 3.68 (3H, s), 2.44 (1H, ddd, J=4.2, 6.6, 8.4 Hz), 1.80 (1H, ddd, J=4.2, 5.0, 8.4 Hz), 1.52 (1H, ddd, J=5.0, 6.6, 7.8 Hz), 1.22 (1H, ddd, J=4.2, 6.6, 8.4 Hz). ¹³C NMR (75 MHz, CDCl₃): δ173.8, 147.8, 146.3, 133.8, 119.7, 108.1, 106.6, 101.0, 51.8, 26.8, 26.1, 23.7, 16.7.

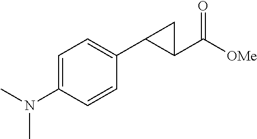 [SMILES: COC(=O)C1CC1c1ccc(N(C)C)cc1]

Methyl trans-2-(4-(dimethylamino)phenyl)cyclopropanecarboxylate: 0.359 g, 81%, dark gold solid. ¹H NMR (300 MHz, CDCl₃): δ6.95 (2H, m), 6.64 (2H, m), 3.66 (3H, s), 2.80 (6H, s), 2.45 (1H, ddd, J=4.2, 4.5, 11.4 Hz), 1.79 (1H, ddd, J=4.2, 4.7, 8.4 Hz), 1.52 (1H, dd, J=4.8, 14.1 Hz), 1.24 (1H, ddd, J=4.5, 6.6, 8.3). ¹³C NMR (75 MHz, CDCl₃): δ174.4, 149.8, 127.9, 127.4, 113.1, 52.0, 41.0, 26.2, 23.8, 16.8.

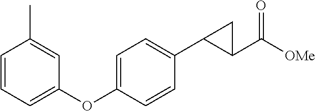 [SMILES: COC(=O)C1CC1c1ccc(Oc2cccc(C)c2)cc1]

Methyl trans-2-(3-(m-tolyloxy)phenyl)cyclopropanecarboxylate: 0.298 g, 100%, dark gold oil. ¹H NMR (300 MHz, CDCl₃): δ6.99 (8H, m), 3.71 (3H, s), 2.48 (1H, ddd), 2.33 (3H, s), 1.88 (1H, ddd), 1.57 (1H, ddd), 1.29 (1H, ddd). ¹³C NMR (75 MHz, CDCl₃): δ130.2, 129.9, 129.7, 129.5, 125.3, 124.4, 121.1, 199.2, 117.1, 116.8, 116.1, 52.2, 26.3, 24.3, 24.2, 17.4, 17.2.

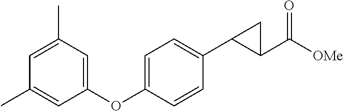 [SMILES: COC(=O)C1CC1c1ccc(Oc2cc(C)cc(C)c2)cc1]

Methyl trans-2-(4-(3,5-dimethylphenoxy)phenyl)cyclopropanecarboxylate: 0.125 g, 95%, gold oil. ¹H NMR (300 MHz, CDCl₃): δ7.03 (2H, m), 6.91 (2H, m), 6.72 (1H, s), 6.59 (2H, s), 3.71 (3H, s), 2.51 (1H, ddd), 2.26 (6H, s), 1.85 (1H, ddd), 1.60 (1H, ddd), 1.29 (1H, ddd). ¹³C NMR (75 MHz, CDCl₃): δ174.1, 157.6, 156.3, 139.8, 134.8, 127.8, 125.2, 119.3, 116.6, 52.2, 26.0, 24.1, 21.6, 17.1.

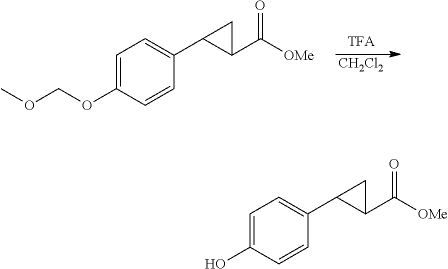

Methyl 2-(4-hydroxyphenyl)cyclopropanecarboxylate: A round bottom flask charged with the methyl ester (0.851 g, 3.6 mmol, 1 eq) in CH₂Cl₂ (20 mL) was brought to 0° C. Trifluoroacetic acid (2 mL, 26 mmol, 7.2 eq) was added slowly. The reaction was stirred for 24 h allowing to warm to rt. The crude reaction mixture was diluted with CH₂Cl₂ (50 mL) and then washed with saturated NaHCO₃ (50 mL) and saturated NaCl (50 mL), dried over anhydrous Na₂SO₄, and concentrated under reduced pressure. The desired methyl 2-(4-hydroxyphenyl)cyclopropanecarboxylate was isolated by flash chromatography over silica gel using 3:1 hexanes:ethyl acetate as a light gold oil in 62% yield (0.239 g). ¹H NMR (300 MHz, CDCl₃): δ6.95 (2H, d, J=8.4 Hz), 6.75 (2H, d, J=8.4 Hz), 3.71 (3H, s), 2.48 (1H, m), 1.82 (1H, m), 1.55 (1H, m), 1.26 (1H, m). ¹³C NMR (75 MHz, CDCl₃): δ174.8, 154.9, 131.8, 127.8, 115.6, 52.2, 36.1, 25.6, 24.0, 17.0.

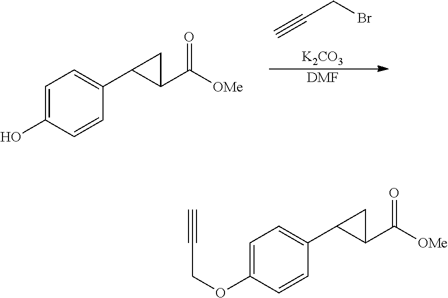

Methyl 2-(4-(prop-2-ynyloxy)phenyl)cyclopropanecarboxylate: An oven-dried round bottom flask charged with anhydrous dimethylformamide (2 mL), methyl 2-(4-hydroxyphenyl)cyclopropanecarboxylate (0.180 g, 0.9 mmol, 1 eq) and anhydrous potassium carbonate (0.418 g, 2.8 mmol, 3 eq) was stirred at 55° C. for 30 min. The reaction mixture was cooled to rt and propargyl bromide (98 pt, 1.1 mmol, 1.2 eq) was added. The reaction was stirred for 5 h at rt. The crude reaction mixture was poured on ice water (25 mL) and no precipitate formed. The organic products were extracted with ethyl acetate (3×30 mL), washed with saturated NH₄Cl (25 mL) and saturated NaCl (25 mL), dried over Na₂SO₄ and concentrated in vacuo affording the desired methyl 2-(4-(prop-2-ynyloxy)phenyl)cyclopropanecarboxylate as a dark yellow oil in 79% yield (0.170 g). ¹H NMR (300 MHz, CDCl₃): δ7.04 (2H, d, J=8.7 Hz), 6.89 (2H, d, J=8.7 Hz), 4.66 (2H, d, J=2.4 Hz), 3.71 (3H, s), 2.50 (2H, m), 1.83 (1H, m), 1.56 (1H, m), 1.27 (1H, m). ¹³C NMR (75 MHz, CDCl₃): δ174.1, 156.5, 133.2, 127.6, 115.2, 78.8, 75.8, 56.1, 52.1, 25.9, 23.9, 16.9.

### Example 6

### Saponification of methylcyclopropanecarboxylates

The following procedure is representative for saponification of the methyl cyclopropanecarboxylates to yield the corresponding carboxylic acids.

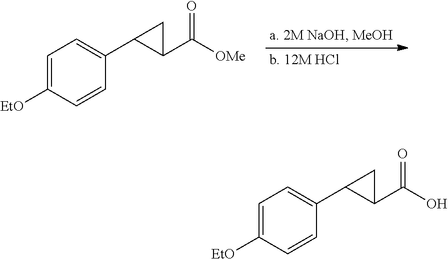 [SMILES: CCOc1ccc(C2CC2C(=O)OC)cc1 ; CCOc1ccc(C2CC2C(=O)O)cc1]

trans-2-(4-ethoxyphenyl)cyclopropanecarboxylic acid: To a solution of methyl trans-2-(4-ethoxyphenyl)cyclopropanecarboxylate (0.2862 g, 1.3 mmol, 1 eq) in methanol (3.4 mL) was added 2 M sodium hydroxide (3.4 mL) while stirring. The reaction was monitored by TLC and upon consumption of the ester, the mixture was poured onto ice (—60 mL) and 12 N HCl (1.4 mL) was added dropwise while stirring. The resulting precipitate was isolated by vacuum filtration. The filter cake was washed with portions of ice water until the filtrate was pH neutral and was dried in vacuo to give trans-2-(4-ethoxyphenyl)cyclopropanecarboxylic acid as an off-white solid in 78% yield (0.210 g). ¹H NMR (400 MHz, CDCl₃): δ7.02 (2H, m), 6.82 (2H, m), 4.00 (2H, q, J=6.8 Hz), 2.56 (1H, ddd, J=4.0, 6.5, 8.3 Hz), 1.82 (1H, ddd, J=4.0, 5.2, 8.3 Hz), 1.61 (1H, quintet, J=5.2 Hz), 1.40 (3H, t, J=6.8 Hz), 1.35 (ddd, J=4.0, 6.5, 8.3). ¹³C NMR (100 MHz, CDCl₃): δ180.1, 158.0, 131.5, 127.7, 114.8, 63.7, 26.9, 23.9, 17.4, 15.0.

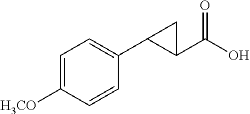 [SMILES: COc1ccc(C2CC2C(=O)O)cc1]

trans-2-(4-methoxyphenyl)cyclopropanecarboxylic acid: 0.237 g, 73%, white solid. ¹H NMR (300 MHz, CD₃OD): δ7.04 (2H, m), 6.82 (2H, m), 4.92 (1H, bs), 3.74 (3H, s), 2.41 (1H, ddd, J=4.2, 6.2, 9.5 Hz), 1.74 (1H, m), 1.47 (1H, quintet, J=4.8 Hz), 1.29 (ddd, J=4.8, 6.2, 8.1). ¹³C NMR (75 MHz, CD₃OD): δ176.1, 158.7, 132.1, 127.1, 113.8, 54.5, 25.5, 23.5, 15.9.

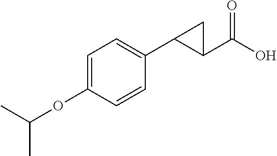 [SMILES: CC(C)Oc1ccc(C2CC2C(=O)O)cc1]

trans-2-(4-isopropoxyphenyl)cyclopropanecarboxylic acid: 0.384 g, 78%; white solid. ¹H NMR (400 MHz, CDCl₃): δ7.02 (2H, m), 6.81 (2H, m), 4.50 (1H, sep, J=6.0 Hz), 2.56 (1H, ddd, J=4.0, 6.8, 8.4 Hz), 1.82 (1H, ddd, J=4.0, 5.2, 8.4 Hz), 1.61 (1H, q, J=5.2 Hz), 1.35 (1H, ddd, J=4.0, 6.8, 8.4 Hz), 1.32 (6H, d, J=6.0 Hz). ¹³C NMR (100 MHz, CDCl₃): δ180.3, 157.0, 131.5, 127.7, 116.2, 70.2, 26.9, 24.0, 22.2, 17.4.

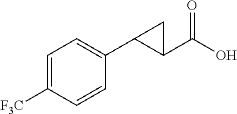 [SMILES: O=C(O)C1CC1c1ccc(C(F)(F)F)cc1]

trans-2-(4-(trifluoromethyl)phenyl)cyclopropanecarboxylic acid: 0.395 g, 98%, pale yellow solid. ¹H NMR (300 MHz, CD₃OD): δ7.56 (2H, d J=8.1 Hz), 731 (2H, d, J=8.1 Hz), 2.54 (1H, ddd, J=4.2, 6.0, 9.0 Hz), 1.93 (1H, ddd, J=4.2, 5.4, 8.4 Hz), 1.59 (1H, m) 1.41 (1H, ddd, J=4.8, 6.3, 8.4 Hz). ¹³C NMR (75 MHz, CD₃OD): δ176.5, 146.5, 127.7, 126.4, 26.6, 25.5, 17.8.

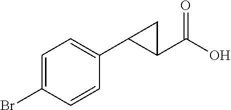 [SMILES: O=C(O)C1CC1c1ccc(Br)cc1]

trans-2-(4-bromophenyl)cyclopropanecarboxylic acid: 0.412 g, 100%, white solid. ¹H NMR (300 MHz, CD₃OD): δ7.40 (2H, d, J=8.1 Hz), 7.04 (2H, d, J=8.1 Hz), 2.43 (1H, ddd, J=4.2, 6.6, 9.0 Hz), 1.83 (1H, m), 1.52 (1H, m), 1.33 (1H, ddd, J=4.5, 6.3, 8.6 Hz). ¹³C NMR (75 MHz, CD₃OD): δ176.7, 141.0, 132.5, 129.0, 121.0, 26.5, 25.1, 17.4.

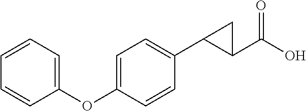 [SMILES: O=C(O)C1CC1c1ccc(Oc2ccccc2)cc1]

trans-2-(4-phenoxyphenyl)cyclopropanecarboxylic acid: 0.148 g, 58%, white solid. ¹H NMR (300 MHz, CD₃OD): δ7.27 (2H, m), 7.04 (3H, m), 6.89 (4H, m), 5.05 (1H, bs), 2.44 (1H, ddd), 1.78 (1H, ddd), 1.50 (1H, ddd), 1.28 (1H, ddd). ¹³C NMR (75 MHz, CD₃OD): δ175.9, 157.6, 156.1, 135.3, 129.7, 127.5, 123.1, 118.9, 118.5, 25.5, 23.8, 16.2.

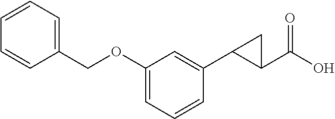 [SMILES: O=C(O)C1CC1c1cccc(OCc2ccccc2)c1]

trans-2-(3-(benzyloxy)phenyl)cyclopropanecarboxylic acid: 0.217 g, 76%, white solid. ¹H NMR (300 MHz, CDCl₃): δ7.30 (5H, m), 7.02 (2H, m), 6.90 (2H, m), 5.03 (2H, s), 2.40 (1H, ddd), 1.74 (1H, ddd), 1.47 (1H, ddd), 1.28 (1H, ddd).

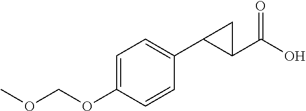 [SMILES: COCOc1ccc(C2CC2C(=O)O)cc1]

2-(4-(methoxymethoxy)phenyl)cyclopropanecarboxylic acid: 0.172 g, 67%, white solid. ¹H NMR (400 MHz, CD₃OD): δ7.01 (2H, d, J=8.6 Hz), 6.90 (2H, d, J=8.6 Hz), 5.09 (2H, s), 4.91 (1H, s), 3.39 (2H, s), 2.38 (1H, ddd, J=4.0, 6.4, 9.2 Hz), 1.72 (1H, ddd, J=4.0, 5.2, 8.1 Hz), 1.45 (1H, ddd, J=4.4, 5.2, 9.2 Hz), 1.26 (1H, ddd, J=4.4, 6.4, 8.1 Hz). ¹³C NMR (100 MHz, CD₃OD): δ176.0, 156.2, 133.2, 133.5, 127.1, 116.3, 94.4, 54.9, 25.5, 23.6, 16.0.

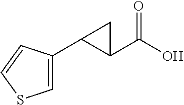 [SMILES: O=C(O)C1CC1c1ccsc1]

trans-2-(thiophen-3-yl)cyclopropanecarboxylic acid: 0.123 g, 90%, yellow solid. ¹H NMR (300 MHz, CD₃OD): δ7.31 (1H, m), 7.08 (1H, m), 6.89 (1H, m), 2.54 (1H, ddd, J=4.2, 6.6, 9.0 Hz), 1.79 (1H, ddd, J=3.9, 5.1, 8.4 Hz), 1.48 (1H, m), 1.30 (1H, ddd, J=4.2, 6.6, 8.4 Hz). ¹³C NMR (75 MHz, CD₃OD): δ177.0, 142.6, 127.0, 126.9, 120.7, 24.5, 22.9, 17.3.

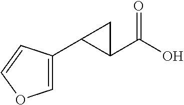 [SMILES: O=C(O)C1CC1c1ccoc1]

trans-2-(furan-3-yl)cyclopropanecarboxylic acid: 0.185 g, 65%, yellow solid. ¹H NMR (300 MHz, CD₃OD): δ7.37 (2H, m), 6.22 (1H, m), 2.28 (1H, ddd, J=3.9, 6.3, 9.3 Hz), 1.70 (1H, ddd, J=4.2, 5.1, 8.4 Hz), 1.42 (1H, m), 1.16 (1H, ddd, J=4.2, 6.3, 8.4 Hz). ¹³C NMR (75 MHz, CD₃OD): δ177.1, 144.4, 140.4, 126.1, 109.8, 23.5, 18.2, 16.5.

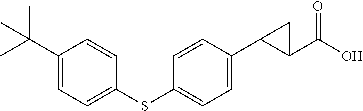 [SMILES: CC(C)(C)c1ccc(Sc2ccc(C3CC3C(=O)O)cc2)cc1]

trans-2-(4-(4-tert-butylphenylthio)phenyl)cyclopropanecarboxylic acid: 0.166 g, 73%, yellow oil. ¹H NMR (300 MHz, CD₃OD): δ7.14 (8H, m), 4.99 (1H, bs), 2.41 (1H, ddd), 1.79 (1H, ddd), 1.49 (1H, ddd), 1.24 (10H, m).

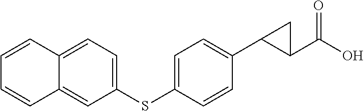 [SMILES: O=C(O)C1CC1c1ccc(Sc2ccc3ccccc3c2)cc1]

trans-2-(4-(naphthalen-2-ylthio)phenyl)cyclopropanecarboxylic acid: 0.203 g, 87%, yellow solid. ¹H NMR (300 MHz, CD₃OD): δ7.72 (4H, m), 7.44 (2H, m), 7.29 (3H, m), 7.13 (2H, m), 2.46 (1H, ddd), 1.84 (1H, ddd), 1.55 (1H, ddd), 1.36 (1H, ddd).

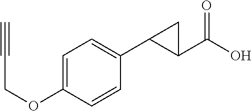 [SMILES: C#CCOc1ccc(C2CC2C(=O)O)cc1]

2-(4-(prop-2-ynyloxy)phenyl)cyclopropanecarboxylic acid: 0.179 g, 70%, white solid. ¹H NMR (300 MHz, CD₃OD): δ7.07 (2H, d, J=8.6 Hz), 6.90 (2H, d, J=8.6 Hz), 4.86 (1H, s), 4.68 (2H, d, J=2.1 Hz)), 2.90 (1H, t, J=2.1 Hz), 2.42 (1H, m), 1.75 (1H, m), 1.48 (1H, m), 1.30 (1H, m).

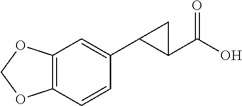 [SMILES: O=C(O)C1CC1c1ccc2c(c1)OCO2]

2-(benzo[d][1,3]dioxol-5-yl)cyclopropanecarboxylic acid: 0.281 g, 74%, white solid. ¹H NMR (300 MHz, CD₃OD): δ6.62 (3H, m), 5.89 (2H, s), 4.95 (1H, bs), 2.39 (1H, ddd, J=3.9, 5.1, 9.0 Hz), 1.73 (1H, ddd, J=5.1, 6.5, 6.9), 1.45 (1H, ddd, J=3.9, 6.5, 9.0 Hz). ¹³C NMR (75 MHz, CD₃OD): 6175.7, 147.9, 146.3, 133.9, 119.3, 107.7, 106.1, 100.9, 25.7, 23.4, 15.8.

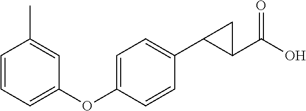 [SMILES: Cc1cccc(Oc2ccc(C3CC3C(=O)O)cc2)c1]

trans-2-(4-(m-tolyloxy)phenyl)cyclopropanecarboxylic acid: 0.199 g, 72%, dark brown oil. ¹H NMR (300 MHz, CD₃OD): δ10.89 (1H, bs), 7.00 (8H, m), 2.54 (1H, ddd), 2.30 (3H, s), 1.84 (1H, ddd), 1.60 (1H, ddd), 1.34 (1H, ddd).

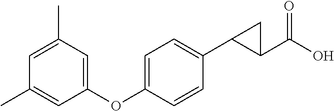 [SMILES: Cc1cc(C)cc(Oc2ccc(C3CC3C(=O)O)cc2)c1]

trans-2-(4-(3,5-dimethylphenoxy)phenyl)cyclopropanecarboxylic acid: 0.048 g, 41%, off-white solid. ¹H NMR (300 MHz, CD₃OD): δ7.09 (2H, m), 6.85 (2H, m), 6.72 (1H, s), 6.54 (2H, s), 4.89 (1H, bs), 2.44 (1H, m), 2.23 (6H, s), 1.77 (1H, m), 1.49 (1H, m), 1.30 (1H, ddd).

### Example 7

### Curtius Rearrangements of Carboxylic Acids to Boc-Protected Amines

The following example is representative for Curtius rearrangement of carboxylic acids to general the corresponding t-butylcarbamate protected amines using diphenylphosphorylazide, triethylamine and t-butanol. In some cases, the carbamate could not be purified completely so impure material was taken on to the subsequent hydrolysis step.

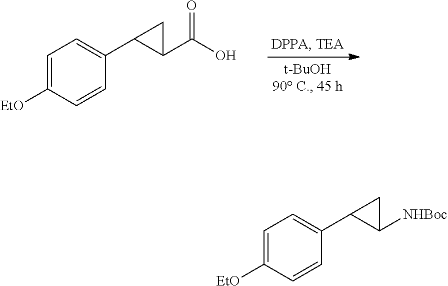 [SMILES: CCOc1ccc(C2CC2C(=O)O)cc1 ; CCOc1ccc(C2CC2NC(=O)OC(C)(C)C)cc1]

### tert-Butyl trans-[2-(4-ethoxyphenyl)cyclopropyl]carbamate

Diphenylphosphorazidate (125 μL, 0.58 mmol, 1.2 eq) and anhydrous triethylamine (94 μL, 0.67 mmol, 1.4 eq) were added sequentially to a room temperature solution of trans-2-(4-ethoxyphenyl)cyclopropanecarboxylic acid (0.100 g, 0.48 mmol, 1 eq) in anhydrous tert-butanol (1 mL). The reaction was heated to 90° C. with an oil bath for 41 h, cooled to rt and concentrated to dryness under reduced pressure. The resulting residue was partitioned between ethyl acetate (10 mL) and 10% aqueous K₂CO₃ (10 mL). The organic products were extracted with ethyl acetate (2×10 mL), dried over Na₂SO₄, filtered, and concentrated in vacuo. The tert-butyl trans-[2-(4-ethoxyphenyl)cyclopropyl]carbamate was isolated by flash chromatography using 5:1 hexanes:ethyl acetate affording a yellow solid in 30% yield (0.040 g). ¹H NMR (400 MHz, CDCl₃): δ7.06 (2H, m), 6.79 (2H, m), 4.85 (1H, bs), 3.99 (2H, q, J=6.8 Hz) 2.64 (1H, bs), 1.98 (1H, m), 1.45 (9H, s), 1.38 (3H, d, J=6.8 Hz), 1.08 (2H, m). ¹³C NMR (100 MHz, CDCl₃): δ157.5, 132.8, 127.9, 114.6, 63.6, 28.6, 24.5, 16.0, 15.0.

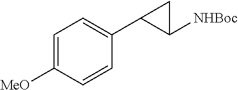 [SMILES: COc1ccc(C2CC2NC(=O)OC(C)(C)C)cc1]

tert-butyl trans-2-(4-methoxyphenyl)cyclopropylcarbamate: 0.186 g, 67%, white solid. ¹H NMR (300 MHz, CDCl₃): δ7.07 (2H, d, J=8.4 Hz), 6.80 (2H, d, J=8.4 Hz), 4.96 (1H, bs), 3.75 (3H, s), 2.64 (1H, m), 1.95 (1H, m), 1.45 (9H, s), 1.07 (2H, m). ¹³C NMR (75 MHz, CDCl₃): δ158.2, 133.2, 128.0, 120.4, 114.0, 79.7, 55.5, 32.4, 28.7, 24.5, 16.0.

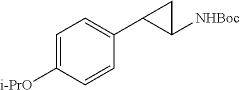

tert-butyl trans-[2-(4-isopropoxyphenyl)cyclopropyl]carbamate: 0.077 g, 57%, white solid. ¹H NMR (400 MHz, CDCl₃): δ7.05 (2H, m), 6.79 (2H, m), 4.89 (1H, bs), 4.48 (1H, sep, J=6.0 Hz), 2.65 (1H, bs), 1.98 (1H, ddd, J=3.2, 6.4, 9.3 Hz), 1.45 (9H, s), 1.30 (6H, d, J=6.0 Hz), 1.08 (2H, m). ¹³C NMR (100 MHz, CDCl₃): δ156.4, 132.8, 127.9, 116.1, 70.2, 32.2, 28.6, 24.4, 22.3, 16.0.

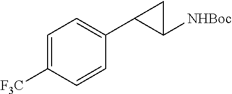 [SMILES: CC(C)(C)OC(=O)NC1CC1c1ccc(C(F)(F)F)cc1]

tert-butyl trans-[2-(4-(trifluoromethyl)phenyl)cyclopropyl]carbamate: 0.056 g, 42%, white solid. ¹H NMR (300 MHz, CDCl₃): δ7.50 (2H, d, J=8.1 Hz), 7.22 (2H, d, J=8.1 Hz), 4.85 (1H, bs), 2.74 (1H, m), 2.09 (1H, m), 1.45 (9H, s), 1.21 (2H, m). ¹³C NMR (75 MHz, CDCl₃): δ156.1, 145.4, 128.8, 126.8, 126.0, 79.3, 32.0, 28.6, 23.1, 16.1.

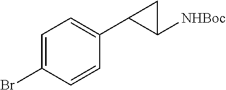 [SMILES: CC(C)(C)OC(=O)NC1CC1c1ccc(Br)cc1]

tert-butyl trans-[2-(4-bromophenyl)cyclpropyl]carbamate: 0.065 g, 50%, white solid. ¹H NMR (300 MHz, CDCl₃): δ7.36 (2H, d, J=8.1 Hz), 7.01 (2H, d, J=8.1 Hz), 4.84 (1H, bs), 2.66 (1H, m), 1.98 (1H, m), 1.45 (9H, s), 1.13 (2H, m). ¹³C NMR (75 MHz, CDCl₃): δ156.5, 140.0, 131.5, 128.6, 119.9, 80.0, 32.8, 28.6, 25.0, 16.4.

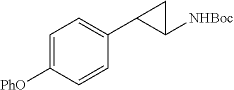 [SMILES: CC(C)(C)OC(=O)NC1CC1c1ccc(Oc2ccccc2)cc1]

tert-butyl trans-[2-(4-phenoxyphenyl)cyclopropyl]carbamate: 0.035 g, 29%, yellow oil. ¹H NMR (300 MHz, CDCl₃): δ7.33 (2H, m), 7.10 (3H, m), 6.93 (4H, m), 4.85 (1H, bs), 2.69 (1H, m), 2.03 (1H, m), 1.46 (9H, s), 1.15 (2H, m). ¹³C NMR (75 MHz, CDCl₃): δ157.9, 156.6, 155.5, 136.0, 129.9, 128.1, 123.2, 119.4, 118.7, 79.9, 32.6, 28.6, 24.8, 16.3.

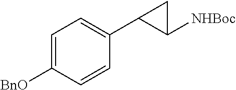 [SMILES: CC(C)(C)OC(=O)NC1CC1c1ccc(OCc2ccccc2)cc1]

tert-butyl trans-[2-(4-(benzyloxy)phenyl)cyclopropyl]carbamate: 0.039 g, 27%, yellow solid. ¹H NMR (300 MHz, CDCl₃): δ7.39 (5H, m), 7.07 (2H, d, J=8.7 Hz), 6.87 (2H, d, J=8.7 Hz), 5.03 (2H, s), 4.82 (1H, bs), 2.65 (1H, m), 1.99 (1H, ddd), 1.45 (9H, s), 1.09 (2H, m). ¹³C NMR (75 MHz, CDCl₃): δ157.5, 137.4, 133.3, 128.8, 128.1, 128.0, 127.7, 115.0, 70.3, 32.4, 29.9, 28.7, 24.6, 16.1.

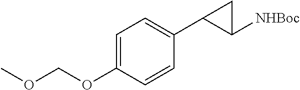 [SMILES: COCOc1ccc(C2CC2NC(=O)OC(C)(C)C)cc1]

tert-butyl 2-(4-(methoxymethoxy)phenyl)cyclopropylcarbamate: 0.144 g, 50% yield, white solid. ¹H NMR (300 MHz, CDCl₃): δ7.04 (2H, m), 6.92 (2H, m), 5.11 (2H, s), 5.05 (1H, bs), 3.43 (3H, s), 2.63 (1H, m), 1.97 (1H, m), 1.44 (9H, s), 1.05 (2H, m). ¹³C NMR (75 MHz, CDCl₃): δ156.6, 155.8, 134.4, 127.9, 116.5, 94.8, 79.7, 56.1, 32.5, 28.6, 24.5, 16.1.

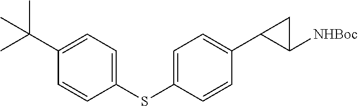 [SMILES: CC(C)(C)OC(=O)NC1CC1c1ccc(Sc2ccc(C(C)(C)C)cc2)cc1]

tert-butyl trans-[2-(4-(4-tert-butylphenylthio)phenyl]carbamate: 0.034 g, 29%, yellow oil. ¹H NMR (300 MHz, CDCl₃): δ7.29 (6H, m), 7.06 (2H, d, J=8.4 Hz), 4.85 (1H, bs), 2.70 (1H, m), 2.01 (1H, m), 1.24 (18H, m), 0.95 (2H, m). ¹³C NMR (75 MHz, CDCl₃): δ150.3, 140.1, 133.4, 131.5, 130.7, 130.3, 127.5, 127.1, 126.4, 34.7, 32.9, 31.5, 29.9, 28.6, 25.1, 16.6.

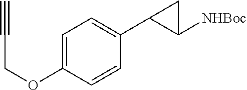 [SMILES: C#CCOc1ccc(C2CC2NC(=O)OC(C)(C)C)cc1]

tert-butyl trans-2-(4-(prop-2-ynyloxy)phenyl)cyclopropylcarbamate: 0.083 g, 37%, white solid. ¹H NMR (300 MHz, CDCl₃): δ7.05 (2H, m), 6.77 (2H, m), 4.90 (1H, bs), 4.64 (2H, d, J=2.4 Hz), 2.64 (1H, m), 2.50 (1H, t, J=2.4 Hz), 2.00 (1H, m), 1.43 (9H, s), 1.09 (2H, m).

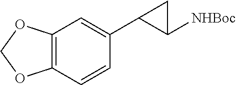 [SMILES: CC(C)(C)OC(=O)NC1CC1c1ccc2c(c1)OCO2]

tert-butyl trans-2-(benzo[d][1,3]dioxol-5-yl)cyclopropylcarbamate: 0.135 g, 43%, off-white solid. ¹H NMR (300 MHz, CDCl₃): δ6.65 (3H, m), 5.87 (2H,$), 4.97 (1H, bs), 2.98 (1H, s), 2.60 (1H, m), 1.95 (1H, m), 1.44 (9H, s), 1.05 (2H, m). ¹³C NMR (75 MHz, CDCl₃): δ156.4, 147.6, 145.8, 134.6, 120.1, 108.0, 100.8, 79.5, 32.2, 28.4, 25.0, 15.8.

### Example 8

### Curtius Rearrangements to Form 2-(Trimethylsilyl)Ethyl Carbamates

The following example is representative for the Curtius rearrangement conditions to form the 2-(trimethylsilyl)ethyl carbamates.

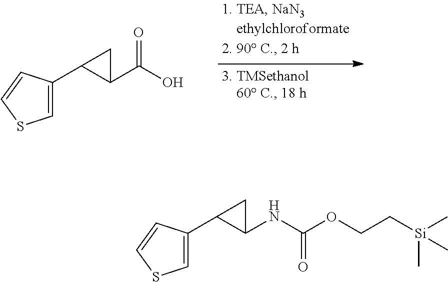 [SMILES: O=C(O)C1CC1c1ccsc1 ; C[Si](C)(C)CCOC(=O)NC1CC1c1ccsc1]

2-(trimethylsilyl)ethyl 2-(thien-3-yl)cyclopropylcarbamate: Ethylchloroformate (80.5 pt, 1.4 eq) and anhydrous triethylamine (103 μL, 1.2 eq) were added sequentially at −10 to −15° C. to a solution of the carboxylic acid (0.101 g, 1 eq) in anhydrous acetone (3.5 mL). The reaction mixture was stirred for 2 h. A solution of NaN₃ (0.065 g, 1.53 eq) in water (190 μL) was added, and the reaction stirred for 2 h. The reaction was quenched with ice cold water (3.5 mL). The acyl azide was extracted with ethyl ether (4×3 mL), dried over anhydrous MgSO₄, and concentrated under reduced pressure. The acyl azide was resuspended in toluene (3.6 mL) and heated to 90° C. while stirred for 2 h to promote the Curtius rearrangement. The reaction mixture was cooled to rt and concentrated under reduced pressure. TMS-ethanol (175 μL) was added and the reaction stirred at 60° C. for 18 h. The excess TMS-ethanol was removed under reduced pressure to afford the desired protected carbamate as a dark amber oil in 93% yield (0.158 g). ¹H NMR (300 MHz, CDCl₃): δ7.21 (m, 1H), 6.90 (m, 2H), 5.10 (bs, 1H), 4.17 (t, J=8.2 Hz, 2H), 2.68 (bs, 1H), 2.05 (m, 1H), 1.11 (m, 2H), 0.98 (m, 2H), 0.02 (s, 9H). ¹³C NMR (300 MHz, CDCl₃): δ141.9, 127.1, 126.5, 125.8, 119.5, 63.4, 60.3, 32.4, 21.1, 18.0, 16.4, −1.2.

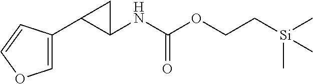 [SMILES: C[Si](C)(C)CCOC(=O)NC1CC1c1ccoc1]

2-(trimethylsilyl)ethyl 2-(furan-3-yl)cyclopropylcarbamate: 0.007 g, 26%, yellow solid. ¹H NMR (300 MHz, CDCl₃): δ7.68 (m, 1H), 7.50 (m, 1H), 7.28 (m, 1H), 6.19 (bs, 1H), 4.20 (m, 2H), 2.58 (bs, 1H), 1.81 (m, 1H), 1.66 (m, 1H), 1.30 (m, 1H), 0.90 (m, 1H), 0.01 (s, 9H). ¹³C NMR (300 MHz, CDCl₃): δ157.4, 144.7, 127.0, 123.9, 123.3, 63.5, 33.5, 20.7, 18.0, 17.4, −1.3.

### Example 9

### Hydrolysis of Boc-Carbamates to Cyclopropylamines

The following example is representative for hydrolysis of the t-butyl carbamates to yield the cyclopropylamines.

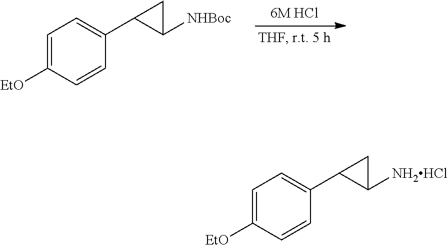

trans-2-(4-ethoxyphenyl)cyclopropylamine hydrochloride: The N-protected carbamate (0.0398 g, 0.14 mmol) was dissolved in THF (0.5 mL) and 6M HCl (0.3 mL). The reaction for stirred at rt for 25 h until TLC indicated complete consumption of starting material. The reaction mixture was concentrated to dryness and the resulting solid residue was dried in vacuo for 24 h over CaSO₄, resulting in a yellow solid in 85% yield (0.031 g). ¹H NMR (300 MHz, CD₃OD): δ7.08 (2H, d, J=8.6 Hz), 6.84 (2H, d J=8.6 Hz), 4.86 (3H, bs), 3.99 (2H, q, J=6.9 Hz), 2.75 (1H, m), 2.24 (1H, m), 2.33 (3H, t, J=6.9 Hz), 1.25 (2H, m). ¹³C NMR (75 MHz, CD₃OD): δ158.2, 130.3, 127.5, 114.5, 63.3, 30.6, 20.7, 14.0, 12.2.

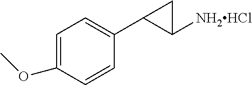

trans-2-(4-methoxyphenyl)cyclopropylamine hydrochloride: 0.131 g, 94%, yellow solid. ¹H NMR (300 MHz, CD₃OD): δ7.06 (2H, d, J=8.4 Hz), 6.83 (2H, d, J=8.4 Hz), 3.75 (3H, s), 2.71 (1H, m), 2.35 (1H, m), 1.36 (1H, m), 1.22 (1H, m). ¹³C NMR (75 MHz, CD₃OD): δ160.0, 132.2, 128.5, 115.0, 55.7, 32.3, 22.5, 14.1.

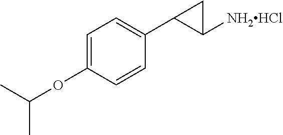

trans-2-(4-isopropoxyphenyl)cyclopropylamine hydrochloride: 0.840 g, 84%, yellow solid. ¹H NMR (400 MHz, CD₃OD): δ7.04 (2H, d, J=8.6 Hz), 6.79 (2H, d, J=8.6 Hz), 4.50 (1H, quintet, J=6.0 Hz), 3.27 (1H, m), 2.71 (1H, m), 2.31 (1H, m), 1.34 (1H, m), 1.23 (6H, d, J=6.0 Hz) 1.19 (1H, m). ¹³C NMR (100 MHz, CD₃OD): δ157.0, 130.4, 127.5, 116.0, 69.8, 30.6, 21.1, 20.7, 12.2.

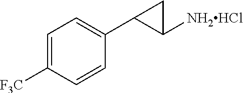

trans-2-(4-(trifluoromethyl)phenyl)cyclopropylamine hydrochloride: 0.044 g, 99%, white solid. ¹H NMR (300 MHz, CD₃OD): δ7.60 (2H, d, J=8.7 Hz), 7.37 (2H, d, J=8.7 Hz), 2.95 (1H, m), 2.50 (1H, m), 1.53 (1H, m), 1.41 (1H, m). ¹³C NMR (75 MHz, CD₃OD): δ144.7, 128.1, 127.5, 126.5, 32.3, 22.3, 14.4.

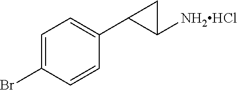

trans-2-(4-bromophenyl)cyclopropylamine hydrochloride: 0.052 g, 99%, white solid. ¹H NMR (300 MHz, CD₃OD): δ7.44 (2H, d, J=8.1 Hz), 7.10 (2H, d, J=8.1 Hz), 2.84 (1H, m), 2.39 (1H, m), 1.46 (1H, m), 1.31 (1H, m). ¹³C NMR (75 MHz, CD₃OD): δ139.2, 132.7, 129.4, 121.3, 32.0, 22.0, 13.9.

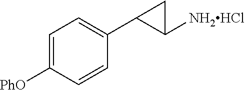

trans-2-(4-phenoxyphenyl)cyclopropylamine hydrochloride: 0.025 g, 88%, yellow solid. ¹H NMR (300 MHz, CD₃OD): δ7.32 (2H, m), 7.17 (2H, m), 7.09 (1H, m), 6.93 (4H, m), 4.87 (3H, s), 2.82 (1H, m), 2.39 (1H, m), 1.42 (1H, m), 1.29 (1H, m). ¹³C NMR (75 MHz, CD₃OD): δ157.5, 156.5, 133.5, 129.7, 127.8, 123.3, 118.8, 118.6, 30.7, 20.8, 12.5.

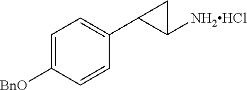

trans-2-(4-(benzyloxy)phenyl)cyclopropylamine hydrochloride: 0.028 g, 87%, yellow solid. ¹H NMR (300 MHz, CD₃OD): δ7.36 (5H, m), 7.09 (2H, d, J=8.7 Hz), 6.93 (2H, d, J=8.7 Hz), 5.05 (2H, s), 4.87 (3H, s), 2.75 (1H, m), 2.32 (1H, m), 1.31 (2H, m). ¹³C NMR (75 MHz, CD₃OD): δ158.0, 137.5, 130.7, 128.3, 127.7, 127.5, 127.3, 115.0, 69.8, 30.6, 20.7, 12.2.

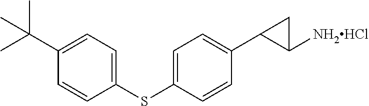

trans-2-(4-(4-tert-butylphenylthio)phenyl)cyclopropylamine hydrochloride: 0.019 g, 66%, yellow solid. ¹H NMR (300 MHz, CD₃OD): δ7.21 (8H, m), 4.86 (3H, s), 2.83 (1H, ddd), 2.35 (1H, ddd), 1.31 (11H, m). ¹³C NMR (75 MHz, CD₃OD): δ137.4, 125.4, 134.2, 131.5, 130.4, 126.9, 126.6, 126.3, 34.3, 30.5, 29.6, 21.0, 12.7.

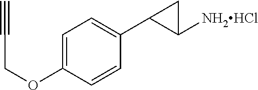

2-(4-(prop-2-ynyloxy)phenyl)cyclopropanamine hydrochloride salt: 0.055 g, 93%, light yellow solid. ¹H NMR (300 MHz, CD₃OD): δ7.12 (2H, d, J=6.9 Hz), 4.87 (3H, s), 4.69 (2H, d, J=2.1 Hz), 2.92 (1H, t, J=2.1 Hz), 2.77 (1H, m), 2.36 (1H, m), 1.39 (1H, m), 1.26 (1H, m). ¹³C NMR (75 MHz, CD₃OD): δ156.7, 131.1, 127.2, 114.8, 78.4, 75.4, 55.3, 30.4, 20.5, 12.1.

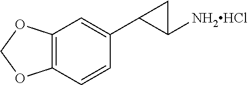

2-(benzo[d][1,3]dioxol-5-yl)cyclopropanamine hydrochloride salt: 0.089 g, 70%, yellow solid. ¹H NMR (300 MHz, CD₃OD): δ6.70 (3H, m), 5.90 (2H, s), 4.86 (3H, s), 2.75 (1H, m), 2.32 (1H, m), 1.37 (1H, m), 1.24 (1H, m). ¹³C NMR (75 MHz, CD₃OD): δ148.0, 146.6, 132.1, 119.5, 107.8, 106.5, 101.0, 30.4, 21.0, 12.1.

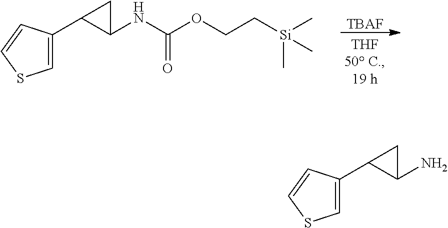

2-(thiophen-3-yl)cyclopropanamine: The 2-(trimethylsilyl)ethylcarbamate was resuspended in a solution of tetra-N-butylammonium fluoride (180 mg, 0.7 mmol, 1.25 eq) in THF (0.7 mL). The reaction was brought to 50° C. and stirred for 19 h. The reaction was quenched by dropwise addition of water (1.8 mL) and stirring for 30 min. The mixture was acidified with 1 M HCl (2 mL), washed with dichloromethane (4×2 mL), alkalinized with aqueous Na₂CO₃, extracted with EtOAC (3×3 mL), dried over K₂CO₃ and concentrated in vacuo. The desired free amine was isolated by flash chromatography using 100:1 chloroform:triethylamine as a yellow oil in 26% yield (0.021 g). ¹H NMR (300 MHz, CDCl₃): δ 7.20 (m, 1H), 6.79 (m, 2H), 2.50 (m, 1H), 1.90 (m, 1H), 0.97 (m, 1H), 0.89 (m, 1H).

### General Information for Biological Analysis

Bioinformatic Analysis. The breast cancer cell line dataset, GSE12777(36) was downloaded from the Gene Expression Omnibus (GEO) at “http://www.ncbi.nlm.nih.gov/geo/”. For expression analysis the CEL files were normalized using R/Bioconductor(40-42) with RMA and individual probe expression values for each gene were obtained. The FAD-dependent amine-oxidase probes were then subset from this dataset, and the expression data was converted into a heatmap using the gplots package. To analyze the expression of FAD-dependent amine-oxidase genes in tumor datasets, we used GSE4922(37), combining both U133A with the U133B chips into a single dataset and normalized as above. For purposes of identifying relative expression within each tumor, the rows consisting of tumor samples were scaled and displayed as a heatmap.

Enzymatic Assays.

LSD1 overexpression, purification and horseradish peroxidase coupled enzyme assay were performed as previously described. (Schmidt, D. M., and McCafferty, D. G. (2007) trans-2-Phenylcyclopropylamine is a mechanism-based inactivator of the histone demethylase LSD1, Biochemistry 46, 4408-4416 (which is incorporated by reference herein); Gooden, D. M., Schmidt, D. M., Pollock, J. A., Kabadi, A. M., and McCafferty, D. G. (2008) Facile synthesis of substituted trans-2-arylcyclopropylamine inhibitors of the human histone demethylase LSD1 and monoamine oxidases A and B, Bioorg. Med. Chem. Lett. 18, 3047-3051 (which is incorporated by reference herein); and Gaweska, H., Henderson Pozzi, M., Schmidt, D. M., McCafferty, D. G., and Fitzpatrick, P. F. (2009) Use of pH and kinetic isotope effects to establish chemistry as rate-limiting in oxidation of a peptide substrate by LSD1, Biochemistry 48, 5440-5445 (which is incorporated by reference herein).

Inhibitors were prepared as 100 mM stocks in dimethylsulfoxide (DMSO) before dilution into assay reagents at appropriate concentrations. Progress curves for time-dependent enzyme inactivation were fit to Equation 1

Product=(vᵢ/k_{obs})*(1−exp^{-kobs*t}) (1)

to obtain values of k_{obs} as a function of inhibitor concentration, which were then fit to Equation 2 to obtain values of k_{inact} and K₁.

k_{obs}=(k_{inact}*[I])/(K_{I}+[I]) (2)

Cell Culture.

MCF7 cells were maintained in DMEM/F12 (Gibco) supplemented with 8% fetal bovine serum (FBS) (Sigma), 1 mM sodium pyruvate and 0.1 mM non-essential amino acids. MDA-MB 231 cells were maintained in DMEM (Cellgro) supplemented with 8% FBS, 1 mM sodium pyruvate and 0.1 mM non-essential amino acids. HCC1937 and HCC1143 cells were maintained in RPMI 1640 (Gibco) supplemented with 8% FBS, 1 mM sodium pyruvate and 0.1 mM non-essential amino acids. All cells were grown in a 37° C. incubator with 5% carbon dioxide.

Transfection Assays.

For siRNA transfections, MCF7 cells were plated in phenol red-free media containing 8% charcoal-stripped FBS (Hyclone laboratories), 1 mM sodium pyruvate and 0.1 mM non-essential amino acids into either 150 mm dishes (for ChIP), 12-well plates (for mRNA levels) or 6-well plates (for Western blot) and were transfected with DharmaFECT 1 (Invitrogen) according to the supplier's protocol. MDA-MB-231 cells were plated in DMEM (Gibco) supplemented with 8% FBS, 1 mM sodium pyruvate and 0.1 mM non-essential amino acids into either 12-well plates (for mRNA levels) or 6-well plates (for Western blot) and were transfected with DharmaFECT 1 (Invitrogen) according to the supplier's protocol.

RNA Isolation and Real-Time PCR.

For RNA analysis, MCF7 cells were seeded in 12-well plates in phenol red-free media containing 8% charcoal-stripped serum, 1 mM sodium pyruvate, and 1 mM non-essential amino acids. After 4 d, the cells were treated with the inhibitors (250 μM). After 6 h, the cells were treated with ethanol (no treatment) or 100 nM E2 for 18 h and then were harvested. Total RNA was isolated using the Aurum Total RNA Mini Kit (Bio-Rad). One half microgram of RNA was reverse transcribed using the iScript cDNA synthesis kit (Bio-Rad). The Bio-Rad iCycler Realtime PCR System was used to amplify and quantify levels of target gene cDNA. qRT-PCR were performed with 8 μL cDNA, 0.4 μM specific primers and iQ SYBR Green Supermix (Bio-Rad). Data are normalized to the 36B4 housekeeping gene and presented as fold induction over control. Data are presented as the mean±SEM for triplicate amplification reactions from one representative experiment. Each experiment was repeated at least three independent times with nearly identical results.

qPCR Primers:

| Primer | Sequence |
|---|---|
| 36B4 F | GGACATGTTGCTGGCCAATAA |
| 36B4 R | GGGCCCGAGACCAGTGTT |
| LSD1 F | GTGCAGTACCTCAGCCCAAAG |
| LSD1 R | CCGAGCCCAGGGATCAG |
| LSD2 F | GCGTGCTGATGTCTGTGATT |
| LSD2 R | GACCTCCTGCTCCTTGAACA |
| pS2 F | TCCCCTGGTGCTTCTATCCTAATAC |
| pS2 R | GCAGTCAATCTGTGTTGTGAGCC |
| PR F | GCATCGTTGATAAAATCCGCAG |
| PR R | AATCTCTGGCTTAGGGCTTGGC |
| GREB1 F | GCAGGCAGGACCAGCTTCTGA |
| GREB1 R | GCTCTGTTCCCACCACCTTGG |
| EGR1 F | CACCTGACCGCAGAGTCTTT |
| EGR1 R | AGCGGCCAGTATAGGTGATG |

### ChIP Assays.

MCF7 cells were grown to 90% confluence in 15 cm dishes in phenol red-free media containing 8% charcoal-stripped FBS, 1 mM sodium pyruvate, and 0.1 mM non-essential amino acids for 3 d, after which the cells were serum starved for 24 h. After treatment with vehicle or E2 (100 mM) for 45 min, the cells were fixed with 1% formaldehyde for 10 min at rt. The reaction was stopped with glycine (250 nM final concentration) by incubation at rt for 5 min. The cells were washed with ice-cold PBS, harvested in PBS, and centrifuged for 1 min. The cells were frozen (˜80° C.) until ready to lyse. The cells were lysed in 1 mL sonication buffer (50 mM HEPES, pH 7.8, 140 mM NaCl, 1 mM EDTA, 1% Triton X-100, 0.1% sodium deoxycholate, 0.1% SDS, 1× protease inhibitor) by sonication (13×13 sec at 9-10 W). The lysate was clarified by centrifugation (15 min, 4° C., 17000×g) and the supernatant collected, diluted with RIPA buffer (50 mM Tris, pH 7.5, 150 mM NaCl, 1% NP-40, 0.5% sodium deoxycholate, 0.05% SDS, 1 mM EDTA, 1× protease inhibitor), and precleared in 100 μL Protein A/G Agarose beads (50% slurry in 10 mM Tris-HCl, pH 8.0, 1 mM EDTA, 200 μg sonicated salmon sperm DNA, and 500 μg Bovine serum albumin) for 30 min at 4° C. Immunoprecipitation was performed for 4-6 hr at 4° C. with antibodies as described below. After immunoprecipitation, 100 μL Protein A/G Agarose beads (50% slurry in PBS) was added and allowed to incubate overnight at 4° C. Precipitates were washed sequentially twice with sonication buffer, buffer A (50 mM HEPES, pH 7.8, 1 mM EDTA, 1% Triton X-100, 0.1% Na-deoxycholate, 0.1% SDS, 1× protease inhibitor), buffer B (20 mM Tris, pH 8.0, 1 mM EDTA, 250 mM LiCl, 0.5% NP-40, 0.5% sodium deoxycholate, 1× protease inhibitor), and TE (10 mM Tris, pH 8.0, 1 mM EDTA). The precipitates were eluted twice with 50 mM Tris, pH 8.0, 1 mM EDTA, 1% SDS at 65° C. for 10 min. Cross-linking was reversed by addition of NaCl (final concentration 230 mM) and incubation overnight at 65° C. Protein was removed by incubation with EDTA (final concentration 4.5 mM) and proteinase K (final concentration 45 μg mL⁻¹) for 1 h at 42° C. DNA was isolated with a QIA-quick PCR Purification kit (Qiagen). qRT-PCR was performed with immunoprecipitated DNA, specific primers, and iQ SYBR Green Supermix (Bio-Rad). Data were normalized to the input for the immunoprecipitation.

### ChIP Primers:

| Primer | Sequence |
|---|---|
| pS2 ERE F | TTAGGCCTAGACGGAATGGGCTTCAT |
| pS2 ERE R | TGAGATTCAGAAAGTCCCTCTTTCCC |
| pS2 distal F | CCAGAGGCCTGGCAGGAAAC |
| pS2 distal R | CGTCCTCTCCACACACCATCTTC |
| PR distal F | TTGGTTCTGCTTCGGAATCTG |
| PR distal R | CCTCCTCTCCTCACTCTTGG |
| PR ERE A F | ATGACATCAGCAGCAGTG |
| PR ERE A R | GAAAGAACACACCAACCTG |
| PR ERE B F | AAATAGGGCAAAGGGAACAG |
| PR ERE B R | CCCACACTTAACCCAATCC |

Cell Viability Assays.

MCF7 cells were seeded at 8000 cells per well in 96 well plates in phenol red-free media containing 8% charcoal-stripped FBS, 1 mM sodium pyruvate, and 0.1 mM non-essential amino acids. MDA-MB-231 cells were seeded at 8000 cells per well in 96 well plates in DMEM media containing 8% FBS, 1 mM sodium pyruvate and 0.1 mM non-essential amino acids. After 4 d, cells were given fresh media and treated with inhibitors at various concentrations (0.2 μM-1 mM) for 24 h. Cells were incubated for 2 h after addition of CellTiter-Blue reagent (Promega), and then the fluorescence was measured (excitation 535 nm, emission 630 nm) using SpectraMax Gemini EM plate (Molecular Devices). The data were background corrected using a ‘no cell’ control.

Cell Cycle Analysis Assays.

MCF7 cells were seeded at 400,000 cells per well in 6 well plates in DMEM/F12 (Invitrogen) containing 8% FBS, 1 mM sodium pyruvate and 0.1 mM non-essential amino acids. After 24 h, the cells were treated with fresh media containing the 250 μM inhibitor. After 48 h of treatment, the cells were pulsed for 2 h with BrdU (10 μg mL⁻¹). The cells were trypsinized and collected using cold IFA buffer (3 mL) (4% charcoal stripped FBS, 150 mM sodium chloride, 10 mM HEPES, pH 7.5). They were washed with PBS, resuspended in 1 mL of PBS and fixed with 70% ethanol. The cells were incubated for 30 min on ice and then stored at −20° C. The cells were washed with PBS containing 0.5% BSA. The pellet was denatured with 2 M hydrochloric acid containing 0.5% BSA. The residual acid was neutralized using 0.1M sodium borate, pH 8.5. The cell pellet was resuspended in dilute anti-BrdU-alexa fluor-488 antibody (Molecular Probes) (PBS+0.5% Tween 20+0.5% BSA+5% antibody). After washing excess away, the cell pellets were resuspended in PI+RNase A (10 μg mL⁻¹ propidium iodide and 10 μg mL⁻¹ RNase A in PBS). The cells were vortexed and analyzed using flow cytometry (Accuri C6).

Cell Proliferation Assays.

MDA-MB 231 or MCF7 cells were seeded at 3000 cells per well in 96-well plates. After 2 d, the cells were treated with fresh media containing the inhibitors at the concentrations indicated. Every 2 d, for a total of 6 or 10 d, respectively, the cells were treated similarly. Total DNA content was measured by fluorescence using Hoechst 33258 dye (Sigma, λₑₓ=360 nm, λₑₘ=460 nm). Data are presented as the mean±SEM for triplicate wells in one representative experiment. Each experiment was repeated at least two independent times, with nearly identical results.

Western Blot Analysis.

MCF7 cells or MDA-MB 231 cells were seeded in six-well plates. Cells were treated after 48 h with inhibitor for 24 h. Whole-cell extracts were isolated using RIPA buffer [50 mM Tris (pH 8.0), 200 mM NaCl, 1.5 mM MgCl₂, 1% Nonidet P-40 (NP40), 1 mM EGTA, 10% glycerol, 50 mM NaF, 2 mM Na₃VO₄ and 1× protease inhibitor mixture]. Crude histones were extracted from the lysate pellet by resuspending in water and precipitating with 25% TCA. The pellets were washed with acetone and then resuspended. Concentration of whole-cell lysate or resuspended histones was determined using Bio-Rad Bradford reagent using BSA for standard curve. For each sample, proteins were resolved by SDS-PAGE and transferred to a PVDF membrane (Biorad).

Antibodies.

H3K4-Me₂ was detected using polyclonal rabbit antibody (Millipore 07-030). H3K9-Me₂ was detected using a monoclonal mouse antibody (Abcam ab1220). Total H3 was detected using a polyclonal rabbit antibody (Abcam ab1791). LSD1 was detected using a monoclonal rabbit antibody (Millipore 05-939) or polyclonal rabbit antibody (Abcam ab17721). ERα (D12) was detected using monoclonal mouse antibody (Santa Cruz sc-8005). GAPDH was detected using polyclonal goat antibody (Santa Cruz sc-20357). Secondary antibodies were purchased from Bio-Rad.

### Example 10

### mRNA Expression Levels of FAD-Dependent Amine Oxidases

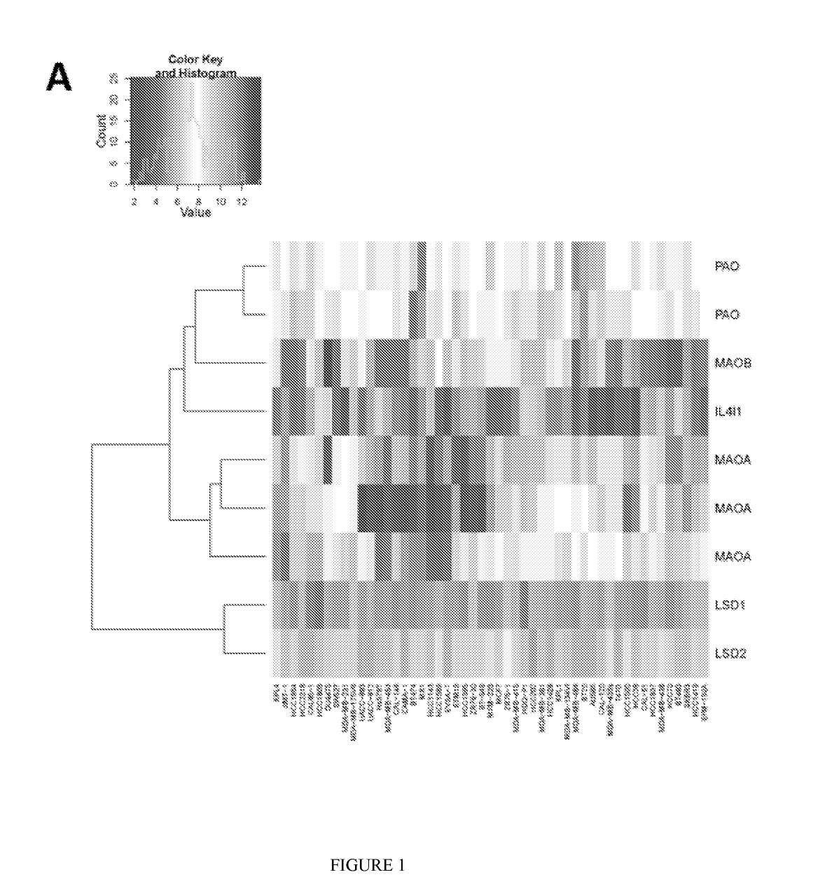

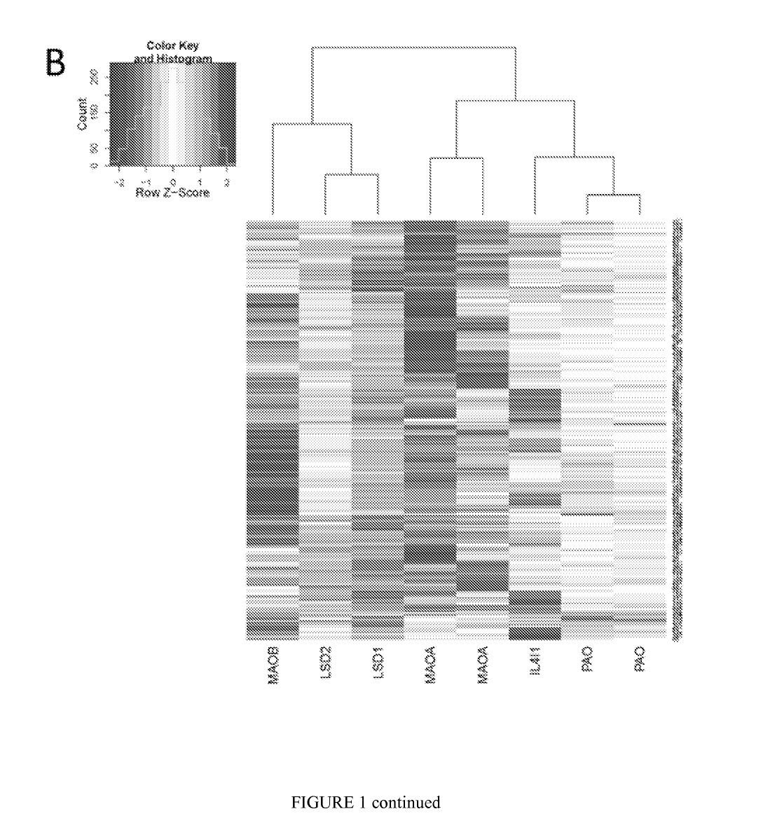

The mRNA expression levels of all of the FAD-dependent amine oxidases were investigated in established cellular models of breast cancer to determine which members of this class of enzymes may be useful targets and to define the best model system(s) to study LSD1 action. To this end, the relative expression levels of nine different FAD-dependent amine oxidases were assessed in published array data derived from a panel of 51 breast cancer cell lines (dataset GSE12777). The data obtained in this manner are presented as a heatmap (FIG. 1A) and indicate that LSD1 and LSD2 are the most highly expressed across all cell lines.

A similar analysis was performed in a breast cancer tumor dataset of 347 primary invasive breast tumors (GSE4922 combining both U133A with the U133B chips). As observed in cell lines, LSD1 and LSD2 were consistently expressed at much higher levels than the other FAD-dependent amine oxidases (FIG. 1B). The high expression levels of LSD1 and LSD2 across all types of breast cancer suggest that, if proven effective, inhibitors of these enzymes may be useful in the treatment of both ER-positive and ER-negative breast cancers.

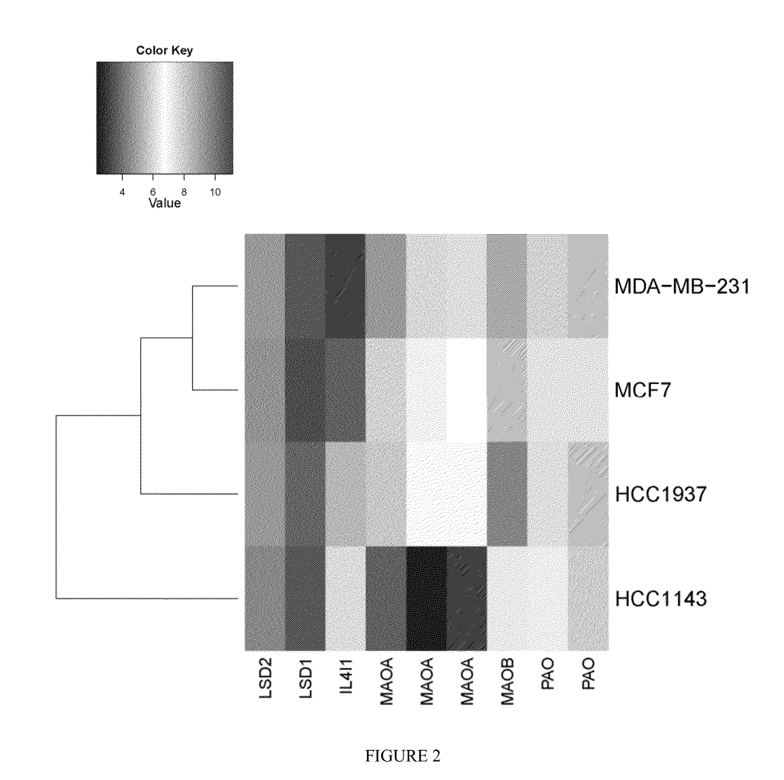

Most significant was the observation that LSD1 was highly expressed in cellular models of the difficult to treat triple negative breast cancers (MDA-MB-231, HCC1143 and HCC1937 cells (FIG. 2)). These expression data indicate that LSD1 is likely to be a useful therapeutic target, and considering expression alone, significant off-target activities on the structurally-related LSD2 enzyme may be observed.

### Example 11

### The Role of LSD1 and LSD2 in Proliferation of ERα-Positive and Triple Negative Breast Cancer Cells

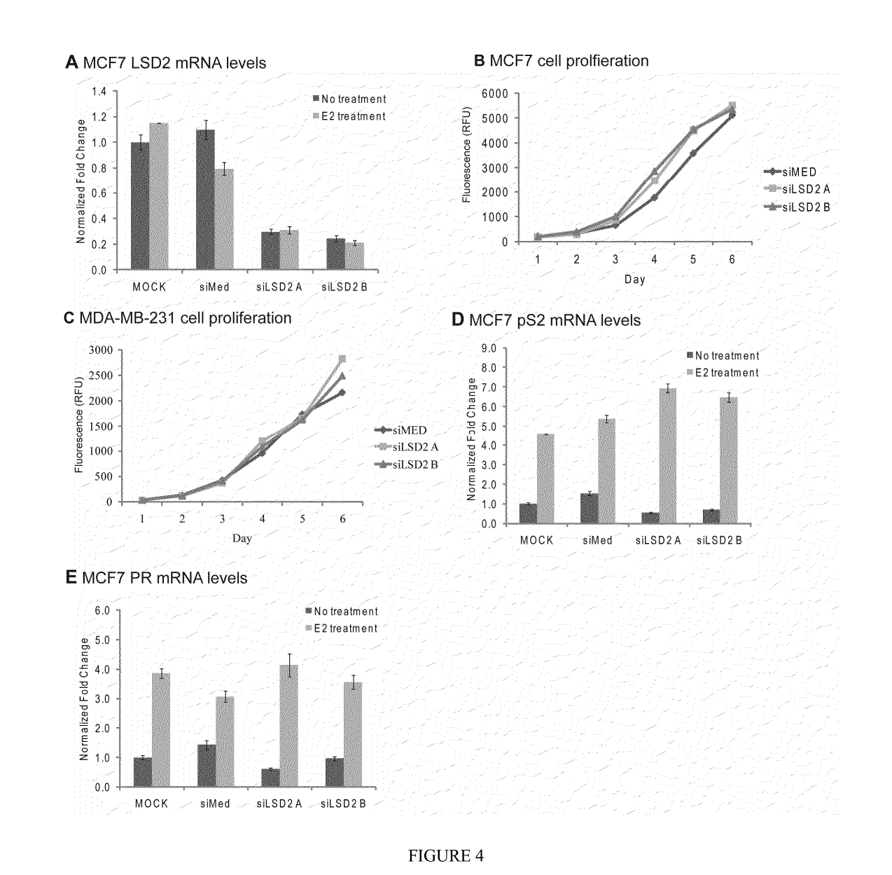

The roles of LSD1 and LSD2 in the proliferation of ERα-positive and triple negative breast cancer cells was assessed following knockdown of their expression using small interfering RNAs (siRNAs). LSD1 and LSD2 were knocked down in both MCF7 and MDA-MB-231 cells using two distinct siRNAs (FIG. 3A-D and FIG. 4A). As shown in FIG. 3E-F, knockdown of LSD1 dramatically inhibited proliferation of both MCF7 and MDA-MB-231 cells, respectively. This was primarily a cytostatic activity. Conversely, knockdown of LSD2 expression using the same approach was without effect on proliferation (FIG. 4B-C). These data suggest that LSD1, but not LSD2, is required for proliferation in these cell models; a result that highlights the utility of targeting this enzyme in breast cancer.

### Example 12

### Inhibition of LSD1 and LSD2

Compounds 1a-1f and 2a-2c were assayed for their ability to inhibit the enzymatic activity of recombinant LSD1 utilizing a horseradish peroxidase coupled assay for the detection of hydrogen peroxide formed in the demethylase catalytic cycle. All of the 2-PCPA-based inhibitors exhibit similar k_{inact} values but the K₁ increased as the steric bulk in the para-position of the aryl ring increased (Table 1, compounds 1a-1f).

TABLE 1
Inhibition kinetics of LSD1 by tranylcypromine and derivatives (1a-f)
and pargyline and derivatives (2a-c).

| | | | | k_{inact}/K_{I} |
|---|---|---|---|---|
| inhibitor | structure | k_{inact} (s⁻¹) | K_{I} (μM) | (M⁻¹ s⁻¹) |
| 2-PCPA | 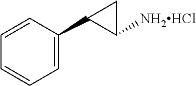 | 0.029 ± 0.004 | 550 ± 150 | 53 |
| 1a | 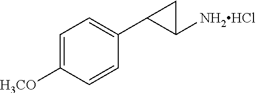 | 0.013 ± 0.001 | 173 ± 37 | 75 |
| 1b | 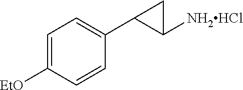 | 0.020 ± 0.002 | 456 ± 86 | 45 |
| 1c | 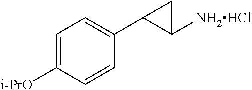 | 0.019 ± 0.002 | 352 ± 76 | 54 |
| 1d | 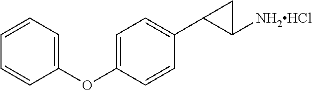 | 0.018 ± 0.002 | 296 ± 77 | 59 |
| 1e | 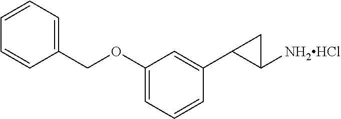 | 0.025 ± 0.005 | 760 ± 280 | 33 |
| 1f | 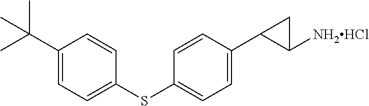 | 0.022 ± 0.003 | 540 ± 160 | 40 |
| pargyline | 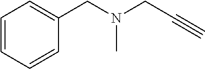 | N.D. | N.D. | <0.2 |
| 2a | 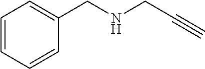 | N.D. | N.D. | <0.6 |
| 2b | 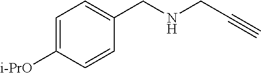 | N.D. | N.D. | <0.8 |
| 2c | 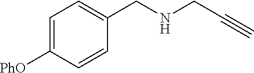 [SMILES: C#CCNCc1ccc(Oc2ccccc2)cc1] | N.D. | N.D. | <0.3 |

Additionally, 2-PCPA and compounds 1a-1d are capable of inactivating MAO B (Table 2).

TABLE 2
Inhibition kinetics of MAO B by tranylcypromine and derivatives (1a-f).

| inhibitor | k_{inact} (s⁻¹) | K_{I} (μM) | k_{inact}/K_{I} (M⁻¹ s⁻¹) |
|---|---|---|---|
| 2-PCPA | 0.024 ± 0.002 | 13.6 ± 3.0 | 1779 |
| 1a | 0.070 ± 0.002 | 86.6 ± 7.8 | 802 |
| 1b | 0.048 ± 0.004 | 63 ± 10 | 762 |
| 1c | 0.046 ± 0.006 | 106 ± 20 | 383 |
| 1d | 0.032 ± 0.002 | 29.5 ± 4.4 | 1095 |
| 1e | not tested | not tested | not tested |
| 1f | not tested | not tested | not tested |

Compounds 1a-1d are not as potent against MAO B as 2-PCPA, with 1c being the least effective in vitro inhibitor of MAO B. Although these compounds are not completely selective for LSD1, progress has been made in decreasing activity against MAO B.

These compounds also have reactivity toward LSD2 as they target covalent inactivation of the flavin following oxidation. In contrast, pargyline and derivatives 2a-2c did not inhibit LSD1 in vitro except at concentrations greater than 5 mM. In fact, treating LSD1 with concentrations of pargyline and derivatives 2a-2c as high as 10 mM only resulted in weak partial inhibition of the enzyme, with greater than 70% of the enzymatic activity remaining after prolonged exposure to the inhibitor.

### Example 13

### Analysis of LSD1 Enzymatic Function in ERα-Dependent Transcription of Genes

Compounds 1a-1c were used as probes of LSD1 function in breast cancer cells. Treatment of MCF7 cells for 24 hours with these compounds (0.2 μM and 1 mM) did not significantly impact viability enabling evaluation of gene expression without the confounding influence of cell death or apoptosis.

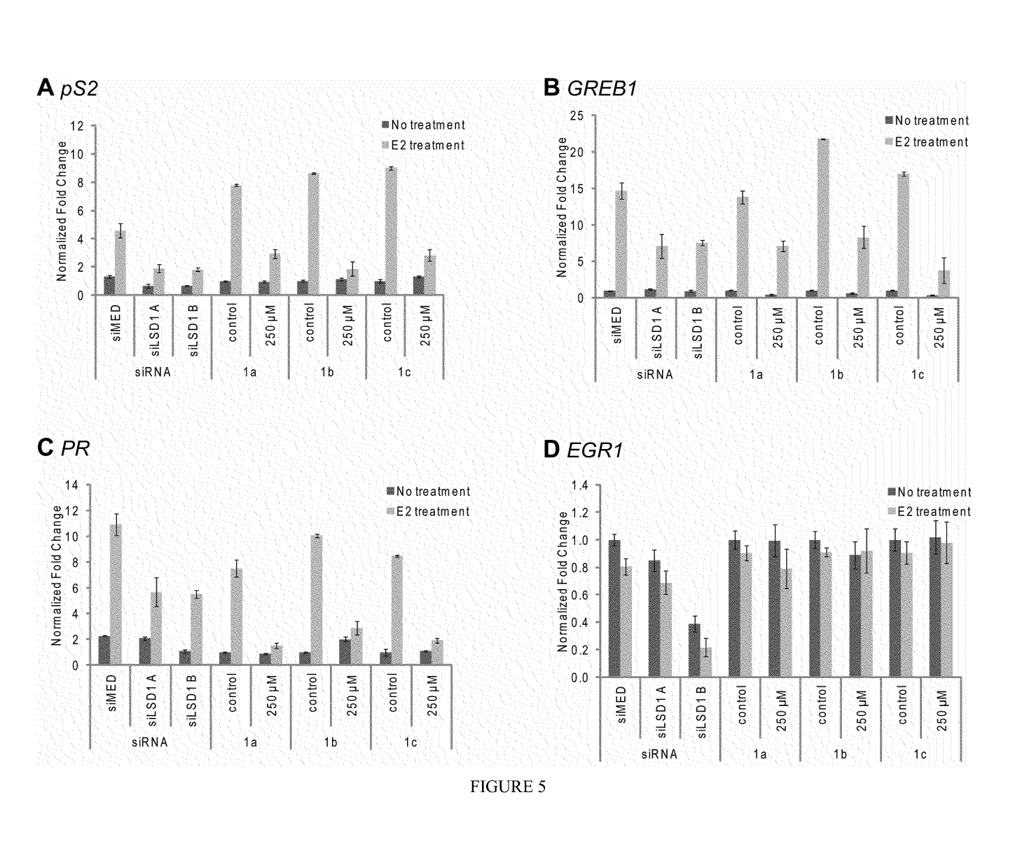

In order to confirm the role of LSD1 in E2-regulated transcription, ERα-positive MCF7 cells were treated with the LSD1 inhibitors or siRNAs to accomplish knockdown of LSD1. Both knockdown (siLSD1) and small molecule inhibition (compounds 1a-1c, 250 μM) of LSD1 resulted in decreased expression of pS2, a marker for hormone-dependent breast cancer, GREB1, a gene important in hormone-responsive cancer and PR, the gene encoding the progesterone receptor (FIG. 5A-C). Similarly, inhibition of the ER-targets genes MCM2, AMyb, CatD, WISP2, SDF1, and Siah2 was also observed. By contrast, other ERα target genes including Erbb4, Smad2, MYC, IL1-R1, and Notch3 were not affected by depletion of LSD1 activity. Additionally, levels of EGR1 (FIG. 5D), an ER unresponsive gene, were not significantly impacted by inhibition or knockdown of LSD1. Treatment with 1a-1c also resulted in changes in basal levels of transcription of some target genes.

Collectively, these data point towards a role of LSD1 in estrogen-independent processes. Interestingly, when MCF7 cells were depleted of LSD2 using siRNA, the mRNA levels of LSD1 and ERα target genes were not changed (FIG. 4D-E). This implies that the LSD1 (and not LSD2) is involved in ER-dependent gene transcription.

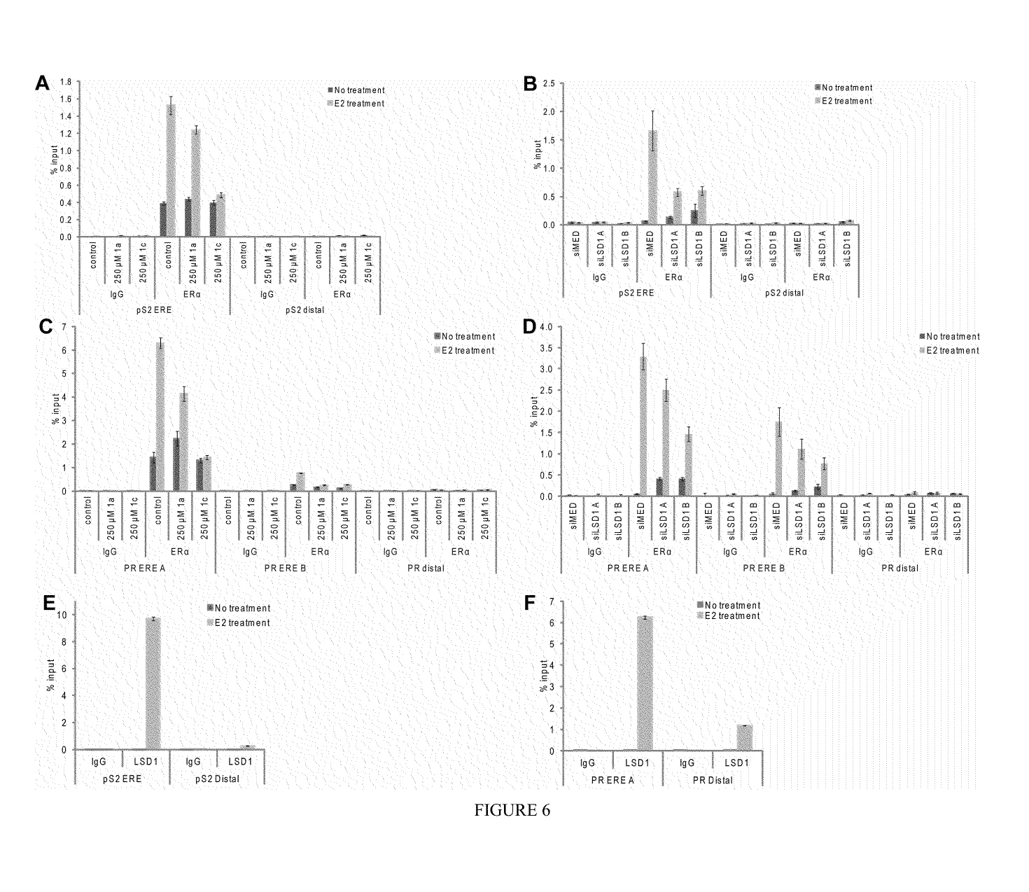

Upon binding estrogen, ERα interacts with specific estrogen response elements (EREs) located within the regulatory regions of target genes where it nucleates the assembly of large multi-protein complexes that influence gene transcription. Using chromatin immunoprecipitation (ChIP) analyzes, it was determined whether recruitment of ERα to target genes was effected by the catalytic demethylase activity of LSD1, or whether LSD1 served as a scaffolding protein. ChIP analysis showed after E2 treatment both ERα (FIG. 6A-B) and LSD1 (FIG. 6E) are clearly recruited to the ERE of pS2. However, when LSD1 expression is ablated using siRNA (FIG. 6B) or the catalytic demethylase activity of the enzyme is inhibited by 1c (FIG. 6A), recruitment of ERα to the pS2 ERE is markedly reduced. This reduction in recruitment coincides with a significant diminution of target gene transcription. This phenomenon is also observed at two validated EREs within the PR promoter (FIG. 6C-D).

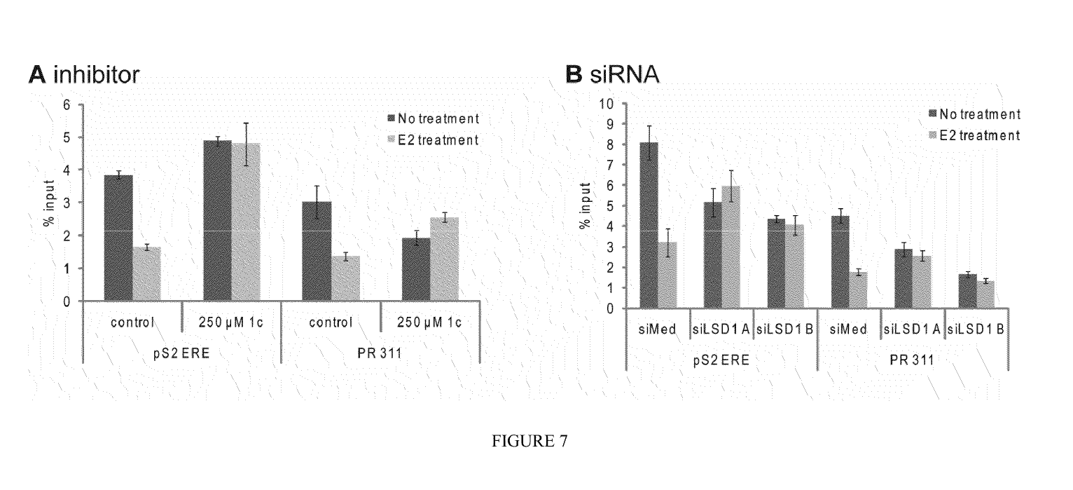

These data suggest that LSD1 may regulate the DNA binding activity of the ERα-transcription factor complex or that LSD 1 dependent modification of chromatin at the target ERE interferes with receptor binding. It has been shown that the methylation status of histones at or close to the pS2 and PR EREs were influenced by LSD1. Specifically, using ChIP, it was determined that E2 treated cells possessed histone H3K4 dimethylation levels that were markedly decreased as compared to untreated cells at the EREs examined. This demethylation event was not observed at histone H3K9. When the catalytic function of LSD1 is inhibited by small molecule inhibitors, or the LSD1 enzyme levels are reduced via siRNA knockdown, E2-induced demethylation of histone H3K4 does not occur. This suggests that LSD1 may be the primary demethylase acting at these sites (see FIG. 7). Although a role for LDS 1 as a scaffold protein has been suggested to be important in ER action, these results confirm that the catalytic activity of LSD1 is also required for the transcriptional activity of ERα at some target genes.

### Example 14

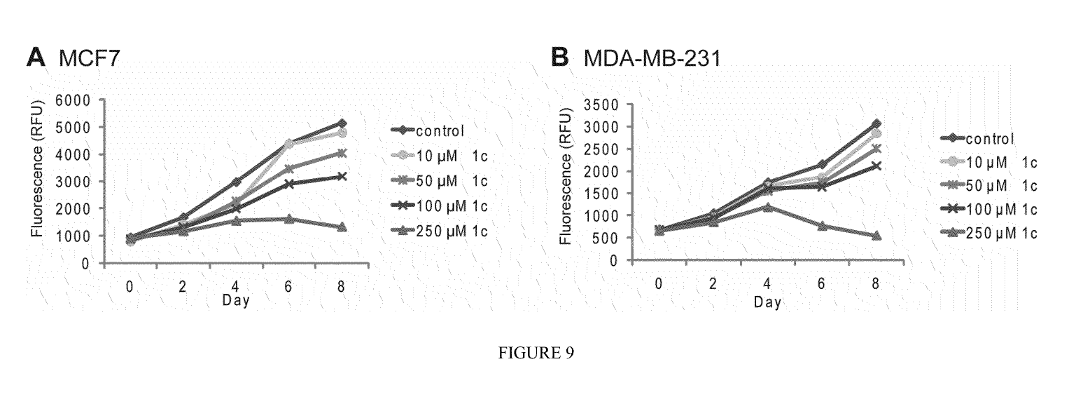

### Inhibition of LSD1 Using 2-PCPA Derivatives or siRNA-Mediated Knockdown Inhibits Breast Cancer Cell Proliferation

Whereas LSD1 is important for estrogen-dependent gene transcription, it was observed that the siRNA-mediated knockdown of LSD1 resulted in a decrease in the proliferation rate of both ER-positive and ER-negative cell lines (FIG. 3C-D). MCF7, MDA-MB-231, HCC1143, and HCC1937 breast cancer cells lines were treated with 250 μM of each of the LSD1 inhibitors 2-PCPA and analogues 1a-1c every other day for 10 days. Similar to what was observed in cells treated with siRNAs directed against LSD1, small molecule LSD1 inhibitors significantly decreased cell proliferation over the course of the 10 day study (FIG. 8A-D). In addition, the reduced rate of cellular proliferation was shown to be inhibitor dose-dependent when examined at concentrations between 10 μM and 250 μM (FIG. 9). Significantly, it was observed that long term treatment with compounds 1b and 1c resulted in cell death between days 6 and 8. Interestingly, inhibitor 1c consistently showed the most dramatic effect on proliferation under all conditions examined. This is intriguing because in vitro this inhibitor did not have the highest inhibitory potency. Without wishing to be bound by theory, the improved efficacy of 1c most likely is due to improved cellular bioavailability of the compound as it is predicted to be more hydrophobic and may have improved translocation across the cellular membrane. Taken together, these studies reveal that inactivation of LSD1 by the small molecule inhibitors significantly influences the proliferation of breast cancer cells, regardless of the levels of ERα.

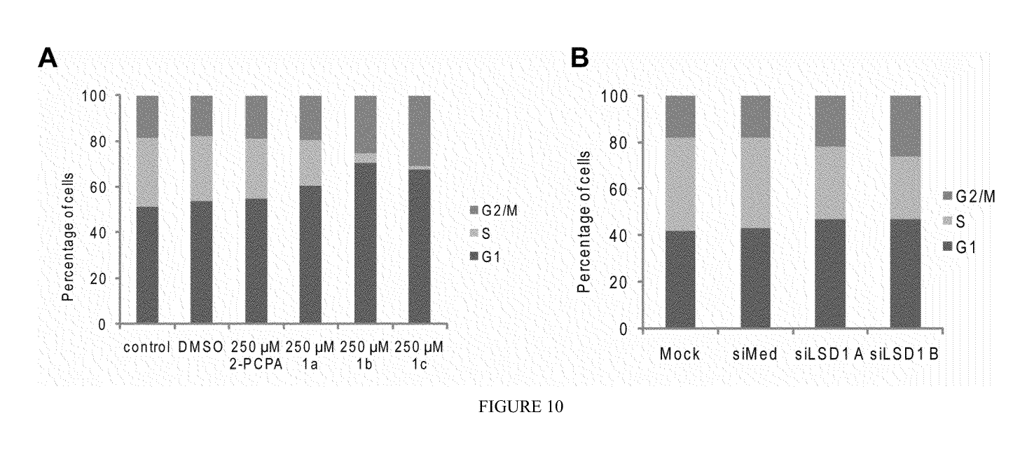

Treatment of either MCF7 or MDA-MB-231 cells for 24 h with 2-PCPA or compounds 1a-1c at drug concentrations up to 500 μM did not induce apoptosis. However, MCF7 cells, treated with 250 μM 2-PCPA or compounds 1a-1c for 24 hours, were growth arrested as evidenced by the accumulation of cells in G1 and G2/M and a decrease in the number of cells in S phase (FIG. 10A). Inhibitor 1c has the most dramatic effect on cell cycle arrest. A similar, albeit less robust, response was observed in MCF7 cells following siRNA-mediated knockdown of LSD1 (FIG. 10B).

### Example 15

### Global Histone H3K4-Me2 is Increased in Breast Cancer Cell Lines after LSD1 Inhibition

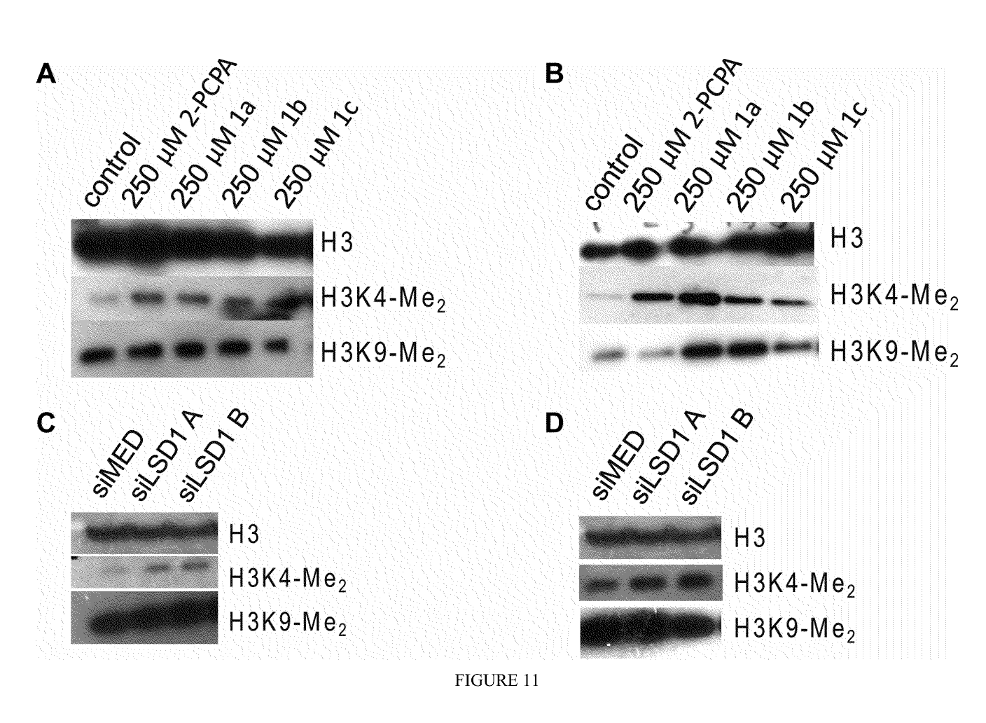

MCF7 and MDA-MB-231 breast cancer cell lines were treated with 2-PCPA and the derivatives 1a-1c, and the levels of H3K4 and H3K9 dimethylation were examined by Western blot analysis. In the ERα-positive MCF7 cells, the level of histone H3K4-Me2 was increased after treatment with 2-PCPA or compounds 1a-1c (FIG. 11A). However, under the same conditions significant changes were not observed in the levels of histone H3K9-Me2. In contrast, robust increases were observed in global dimethylation of both histone H3K4 and histone H3K9 in MDA-MB-231 cells following treatment with the 2-PCPA and compounds 1a-1c (FIG. 11B). Interestingly, siRNA mediated knockdown of LSD 1 expression resulted in an increase in global H3K4-Me2 levels in both cell lines although the level of H3K9-Me2 levels were unchanged (FIG. 11C-D). These results highlight a previously unappreciated complexity in the mechanisms that impact the activity and or target gene specificity of LSD1 action. Inhibition of LSD1 using either 2-PCPA or siLSD1 resulted in an increase in histone H3K4-Me2 but not histone H3K9-Me2. However, increases in both marks were observed in MDA-MB-231 cells.

Although the disclosure above has been described in terms of various aspects and specific embodiments, it is not so limited. A variety of suitable alterations and modifications for operation under specific conditions will be apparent to those skilled in the art. It is therefore intended that the following claims be interpreted as covering all such alterations and modifications as fall within the spirit and scope of the invention.

All patents, publications and references cited herein are hereby fully incorporated by reference. In case of conflict between the present disclosure and incorporated patents, publications and references, the present disclosure should control.

1. A method of treating breast cancer in a subject in need of treatment, comprising administering to the subject an effective amount of a compound of formula (I): 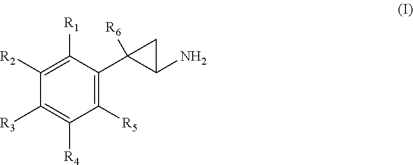
wherein R₁, R₂, R₃, R₄ and R₅ are independently selected from hydrogen, C₁₋₇ alkyl, C₃₋₂₀ heterocyclyl, C₅₋₂₀ aryl, C₁₋₇ alkoxy, C₁₋₇ haloalkyl, halo, amino, cyano, nitro, ether and thioether, or any two of R₁, R₂, R₃, R₄ and R₅ may be taken together with the carbon atoms to which they are attached to form an optionally substituted ring; and
R₆ is selected from hydrogen and optionally substituted C₅₋₂₀ aryl;
or an isomer, prodrug or pharmaceutically acceptable salt thereof.

2. The method of claim 1, wherein R₁, R₂, R₄ and R₅ are hydrogen.

3. The method of claim 1, wherein R₃ is selected from C₁₋₇ haloalkyl, halo, C₁₋₇ alkoxy, and C₅₋₂₀ aryloxy.

4. The method of claim 1, wherein R₂ and R₃ are taken together with the carbon atoms to which they are attached to form a C₃₋₂₀ heterocyclyl ring.

5. The method of claim 4, wherein R₂ and R₃ are taken together to form a five-membered heterocyclyl ring.

6. The method of claim 1, wherein R₆ is hydrogen.

7. The method of claim 1, wherein the compound of formula (I) is selected from the group consisting of: 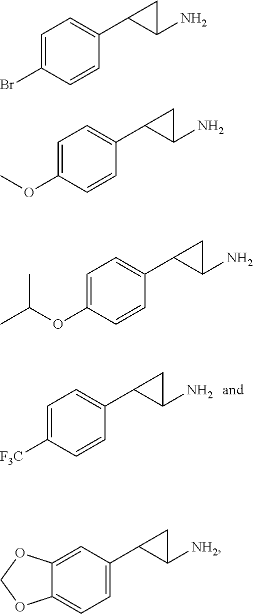
or a pharmaceutically acceptable salt thereof.

8. A method of treating breast cancer in a subject in need of treatment, comprising administering to the subject an effective amount of a compound of formula (II): 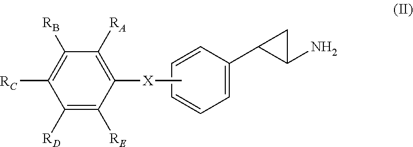
wherein:
X is selected from a bond, O, S, and NH; and
R_{A}, R_{B}, R_{C}, R_{D} and R_{E} are independently selected from hydrogen, C₁₋₇ alkyl, C₅₋₂₀ aryl, C₃₋₂₀ heterocyclyl, C₁₋₇ alkoxy, amino, cyano, nitro, halo, haloalkyl, ether and thioether;
or an isomer, prodrug or pharmaceutically acceptable salt thereof.

9. The method of claim 8, wherein the compound is of formula (III):

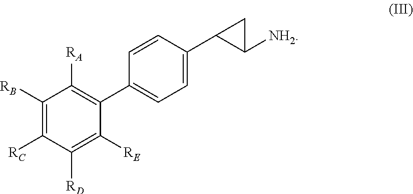

10. The method of claim 8, wherein the compound is of formula (IV):

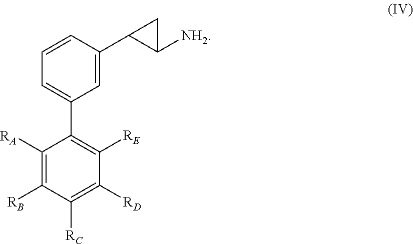

11. The method of claim 8, wherein the compound is of formula (V):

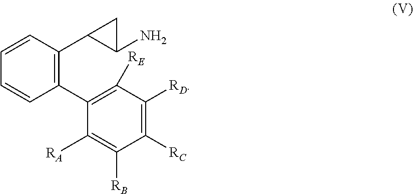

12. The method of claim 8, wherein the compound is of formula (VI): 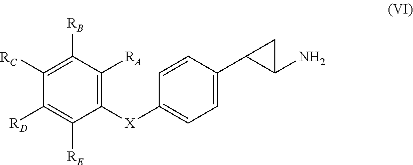
wherein X is selected from O, S, and NH.

13-16. (canceled)

17. The method of claim 8, wherein the compound is of formula (VII): 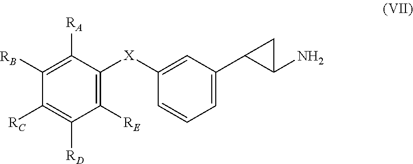
wherein X is selected from O, S, and NH.

18. The method of claim 8, wherein the compound is of formula (VIII): 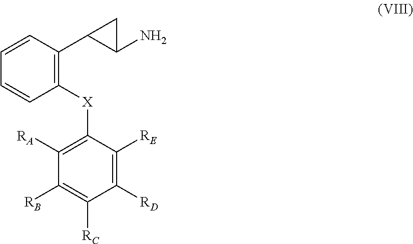
wherein X is selected from O, S, and NH.

19. A method of treating breast cancer in a subject in need of treatment, comprising administering to the subject an effective amount of a compound of formula (IX): 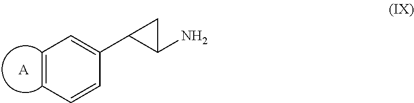
wherein:
A is a C₅-C₆ aryl, cycloalkenyl or heterocyclyl ring;
or an isomer, prodrug or pharmaceutically acceptable salt thereof.

20. The method of claim 19, wherein A is a heterocyclyl ring.

21. The method of claim 19, wherein A is a bicyclic heterocyclyl ring.

22. The method of claim 19, wherein the compound is of formula (X): 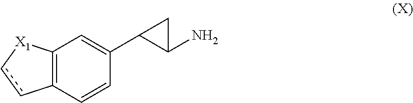
wherein X₁ is selected from CH₂, O, S, and NH; and
- - - represents the presence or absence of a bond.

23. The method of claim 19, wherein the compound is of formula (XI): 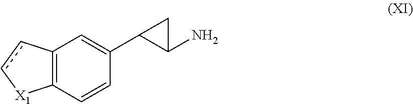
wherein X₁ is selected from CH₂, O, S, and NH; and
- - - represents the presence or absence of a bond.

24. The method of claim 19, wherein the compound is of formula (XII): 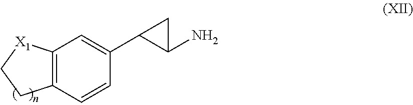
wherein X₁ is selected from CH₂, O, S, and NH;
n is 1 or 2; and
- - - represents the presence or absence of a bond.

25. The method of claim 19, wherein the compound is of formula (XIII): 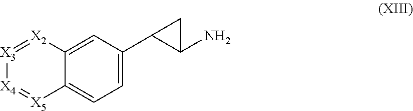
wherein X₂, X₃, X₄ and X₅ are independently selected from CH and N.

26. The method of claim 19, wherein the compound is of formula (XIV): 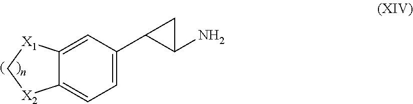
wherein X₁ and X₂ are independently selected from O and S; and
n is 1 or 2.

27-28. (canceled)

29. A method of treating breast cancer in a subject in need of treatment, comprising administering to the subject an effective amount of a compound of formula (XV): 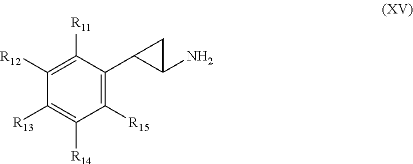
wherein R₁₁, R₁₂, R₁₃, R₁₄ and R₁₅ are independently selected from hydrogen, C₁₋₇ alkyl, C₃₋₂₀ heterocyclyl, C₂₋₇ alkoxy, amino, cyano, nitro, ether and thioether; or an isomer, prodrug or pharmaceutically acceptable salt thereof.

30-35. (canceled)

36. The method of claim 1 wherein the breast cancer is estrogen-receptor negative.

37. A method of reducing proliferation of breast cancer cells, comprising contacting the breast cancer cells with an effective amount of a compound of formula (I): 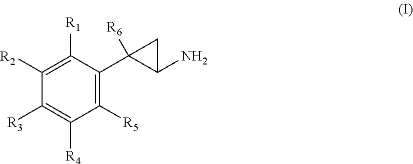
wherein R₁, R₂, R₃, R₄ and R₅ are independently selected from hydrogen, C₁₋₇ alkyl, C3_{—}20 heterocyclyl, C₅₋₂₀ aryl, C₁₋₇ alkoxy, C₁₋₇ haloalkyl, halo, amino, cyano, nitro, ether and thioether, or any two of R₁, R₂, R₃, R₄ and R₅ may be taken together with the carbon atoms to which they are attached to form an optionally substituted ring; and
R₆ is selected from hydrogen and optionally substituted C₅₋₂₀ aryl;
or an isomer, prodrug or pharmaceutically acceptable salt thereof.

38-71. (canceled)

72. The method of claim 37 wherein the breast cancer cell is estrogen-receptor negative.

73. The method of claim 1, wherein the compound is administered in combination with another breast cancer therapy.

74. The method of claim 73, wherein the cancer therapy is radiation.

75. The method of claim 73, wherein the cancer therapy is an anti-cancer agent.

76. The method of claim 73, wherein the cancer therapy is surgery.

77. The method of claim 37, wherein the compound is administered in combination with another breast cancer therapy.

78. The method of claim 77, wherein the cancer therapy is radiation.

79. The method of claim 77, wherein the cancer therapy is an anti-cancer agent.

80. The method of claim 77, wherein the cancer therapy is surgery.


SEQUENCE LISTING (24 sequences)

SEQ ID NO: 1 - DNA, 21 bp, Artificial Sequence
  Primer 36B4 F
ggacatgttgctggccaataa

SEQ ID NO: 2 - DNA, 18 bp, Artificial Sequence
  Primer 36B4 R
gggcccgagaccagtgtt

SEQ ID NO: 3 - DNA, 21 bp, Artificial Sequence
  Primer LSD1 F
gtgcagtacctcagcccaaag

SEQ ID NO: 4 - DNA, 17 bp, Artificial Sequence
  Primer LSD1 R
ccgagcccagggatcag

SEQ ID NO: 5 - DNA, 20 bp, Artificial Sequence
  Primer LSD2 F
gcgtgctgatgtctgtgatt

SEQ ID NO: 6 - DNA, 20 bp, Artificial Sequence
  Primer LSD2 R
gacctcctgctccttgaaca

SEQ ID NO: 7 - DNA, 25 bp, Artificial Sequence
  Primer pS2 F
tcccctggtgcttctatcctaatac

SEQ ID NO: 8 - DNA, 23 bp, Artificial Sequence
  Primer pS2 R
gcagtcaatctgtgttgtgagcc

SEQ ID NO: 9 - DNA, 22 bp, Artificial Sequence
  Primer PR F
gcatcgttgataaaatccgcag

SEQ ID NO: 10 - DNA, 22 bp, Artificial Sequence
  Primer PR R
aatctctggcttagggcttggc

SEQ ID NO: 11 - DNA, 21 bp, Artificial Sequence
  Primer GREB1 F
gcaggcaggaccagcttctga

SEQ ID NO: 12 - DNA, 21 bp, Artificial Sequence
  Primer GREB1 R
gctctgttcccaccaccttgg

SEQ ID NO: 13 - DNA, 20 bp, Artificial Sequence
  Primer EGR1 F
cacctgaccgcagagtcttt

SEQ ID NO: 14 - DNA, 20 bp, Artificial Sequence
  Primer EGR1 R
agcggccagtataggtgatg

SEQ ID NO: 15 - DNA, 26 bp, Artificial Sequence
  Primer pS2 ERE F
ttaggcctagacggaatgggcttcat

SEQ ID NO: 16 - DNA, 26 bp, Artificial Sequence
  Primer pS2 ERE R
tgagattcagaaagtccctctttccc

SEQ ID NO: 17 - DNA, 20 bp, Artificial Sequence
  Primer pS2 distal F
ccagaggcctggcaggaaac

SEQ ID NO: 18 - DNA, 23 bp, Artificial Sequence
  Primer pS2 distal R
cgtcctctccacacaccatcttc

SEQ ID NO: 19 - DNA, 21 bp, Artificial Sequence
  Primer PR distal F
ttggttctgcttcggaatctg

SEQ ID NO: 20 - DNA, 20 bp, Artificial Sequence
  Primer PR distal R
cctcctctcctcactcttgg

SEQ ID NO: 21 - DNA, 18 bp, Artificial Sequence
  Primer PR ERE A F
atgacatcagcagcagtg

SEQ ID NO: 22 - DNA, 19 bp, Artificial Sequence
  Primer PR ERE A R
gaaagaacacaccaacctg

SEQ ID NO: 23 - DNA, 20 bp, Artificial Sequence
  Primer PR ERE B F
aaatagggcaaagggaacag

SEQ ID NO: 24 - DNA, 19 bp, Artificial Sequence
  Primer PR ERE B R
cccacacttaacccaatcc